(19) United States
(12) Patent Application Publication
(10) Pub. No.: US 2025/0257135 A1
(43) Pub. Date: Aug. 14, 2025

(54) NOVEL ANTIBODIES AND NUCLEOTIDE SEQUENCES, AND USES THEREOF

(71) Applicants: BIOINVENT INTERNATIONAL AB, Lund (SE); TRANSGENE SA, Illkirch-Graffenstaden Cedex (FR)

(72) Inventors: Björn FRENDÉUS, Lund (SE); Ingrid TEIGE, Lund (SE); Monika SEMMRICH, Malmö (SE); Linda MÅRTENSSON, Bjärred (SE); Petra HOLMKVIST, Kävlinge (SE); Jean-Baptiste MARCHAND, Obernai (FR); Nathalie SILVESTRE, Ergersheim (FR)

(73) Assignees: BIOINVENT INTERNATIONAL AB, Lund (SE); TRANSGENE SA, Illkirch-Graffenstaden Cedex (FR)

(21) Appl. No.: 19/098,185

(22) Filed: Apr. 2, 2025

Related U.S. Application Data
(62) Division of application No. 17/272,740, filed on Mar. 2, 2021, now Pat. No. 12,291,569.

(30) Foreign Application Priority Data
Sep. 3, 2018 (EP) 18192311.1

(51) Int. Cl. C07K 16/28; A61K 39/00; A61P 35/00; C12N 7/00; C12N 15/86

(52) U.S. Cl. CPC C07K 16/2818; A61P 35/00; C12N 7/00; C12N 15/86; A61K 2039/505; C07K 2317/33; C07K 2317/565; C07K 2317/732; C07K 2317/76; C12N 2710/24143

(57) ABSTRACT

Described are novel anti-CTLA-4 antibody molecules and nucleotide sequences and expression vectors, such as viruses, encoding such antibody molecules. The novel antibody molecules are Treg depleting antibody molecules, and they have an improved depleting effect on CTLA-4 positive cells, such as Tregs, compared to ipilimumab. Describes is also the use of such antibody molecules or nucleotide sequences or viruses in medicine, such as in the treatment of cancer, such as solid tumours.

### CROSS-REFERENCE TO RELATED APPLICATIONS

This application is a divisional application of U.S. patent application Ser. No. 17/272,740, filed Mar. 2, 2021, which is a national phase application under 35 U.S.C. § 371 of International Application No. PCT/EP2019/073488, filed Sep. 3, 2019, which claims priority to European Application No. 18192311.1, filed Sep. 3, 2018. The entire text of each of the above referenced disclosures is specifically incorporated herein by reference.

### INCORPORATION OF SEQUENCE LISTING

The sequence listing that is contained in the file named “EPCLP0092USD1.xml”, which is 87,327 bytes (as measured in Microsoft Windows) and was created on Apr. 1, 2025, is filed herewith by electronic submission and is incorporated by reference herein.

### FIELD OF THE INVENTION

The present invention relates to novel anti-CTLA-4 antibody molecules, nucleotide sequences and expression vectors (e.g. oncolytic virus) encoding such antibody molecules to use thereof in cancer therapy. The novel antibodies have improved Treg depletion compared to ipilimumab.

### BACKGROUND OF THE INVENTION

Cytotoxic T lymphocyte-associated antigen (CTLA-4 or CTLA4), also known as CD152, is a B7/CD28 family member that blocks T cell activation. CTLA-4 is expressed on activated T cells and transmits an inhibitory signal to T cells. It is homologous to the T cell co-stimulatory protein CD28, and both CTLA-4 and CD28 bind to CD80 (also denoted B7-1) and CD86 (also denoted B7-2). CTLA4 is also found in regulatory T cells (Tregs) and contributes to its inhibitory function. The CTLA-4 protein contains an extracellular V domain, a transmembrane domain, and a cytoplasmic tail.

Antibodies that block the interaction of CTLA-4 with its ligands B7.1 and B7.2 can enhance immune responses and have been shown to be capable of stimulating potent anti-tumour immunity (Korman et al 2006, Checkpoint blockade in cancer immunotherapy, Adv Immunol. 90:297-339).

Promising clinical results with immunomodulatory monoclonal antibodies (mAbs) have revived the belief that the immune system holds the key to controlling cancer. The classification of these mAb into checkpoint blockers (antagonists) or activators of co-stimulatory molecules (agonists) has recently come into question with the finding that examples of both types may combat tumours through depletion of suppressive regulatory T cells (Treg).

Immunomodulatory mAbs, such as ipilimumab and other anti-CTLA4 antibodies, have shown positive outcomes when trialled in difficult-to-treat malignancies, albeit in a minority of patients (Hodi, F. S., et al. 2010, N Engl J Med 363 (8): 711-723; Beatty, G. L., et al. 2011, Science 331 (6024): 1612-1616; Brahmer, J. R., et al. 2012, N Engl J Med 366 (26): 2455-2465; Topalian, S. L., et al. 2012 N Engl J Med 366 (26): 2443-2454). These promising results have helped to reinvigorate the belief that the immune system can hold the key to controlling cancer. These mAbs were generated to target key molecular regulators on T cells or antigen-presenting cells (APCs) and to boost anti-cancer immunity through blockade of inhibitory signals (checkpoint blockers) or delivery of co-stimulatory signals (agonists). Recently this binary classification has come into question when the therapeutic activity of anti-CTLA4 antibodies, anti-GITR antibodies and anti-OX40 antibodies, which all target T cells, was found to involve deletion of suppressive CD4+ T regulatory cells dependent on co-engagement of activatory FcγRs (Bulliard, Jolicoeur et al. 2013, Marabelle, A., et al. 2013, J Clin Invest 123 (6): 2447-2463; Simpson, T. R., et al. J Exp Med 210 (9): 1695-1710).

A monoclonal CTLA-4 antibody, ipilimumab (YERVOY®; earlier denoted 10D1, BMS-734016, MDX 101, MDX-010, MDX-CTLA-4 and MDX-CTLA4), has been approved in several countries for the treatment of melanoma and is undergoing clinical trials for other indications (Weber 2008, Overcoming immunologic tolerance to melanoma: targeting CTLA-4 with ipilimumab (MDX-010) Oncologist, 13 (Suppl 4): 16-25). Ipilimumab is a fully human anti-CTLA-4 monoclonal antibody (IgG1K) produced in Chinese hamster ovary cells by recombinant DNA technology. It has 477202-00-9 and 6T8C155666. Ipilimumab is further defined in U.S. Pat. No. 9,789,182, which also sets out the sequences of ipilimumab's heavy and light chains (as SEQ ID NOs: 17 and 18, respectively), the sequences of the VH and/or VL regions (as SEQ ID NOs: 19 and SEQ ID NO:20, respectively) and the CDR sequences (heavy chain CDR1, CDR2 and CDR3 as set forth in SEQ ID NOs: 21, 22, and 23, and light chain CDR1, CDR2 and CDR3 as set forth in SEQ ID NOs: 24, 25, and 26).

A second fully human monoclonal anti-CTLA-4 antibody that has been tested in several clinical trials is tremelimumab (formerly ticilimumab, CP-675,206) (Ribas 2008, Overcoming immunologic tolerance to melanoma: targeting CTLA-4 with tremelimumab (CP-675,206) Oncologist, 13 (Suppl 4): 10-5; Callahan et al 2010, Anti-CTLA-4 Antibody Therapy: Immune Monitoring During Clinical Development of a Novel Immunotherapy. Semin Oncol. 37 (5): 473-484.; Blank et al 2015, Therapeutic use of anti-CTLA-4 antibodies. International Immunology, 27 (1): 3-10).

Anti-CTLA-4 antibodies have been described in several patent applications and patents, including the following.

WO 93/00431 refers to a CTLA4 receptor protein, to a CTLA4Ig fusion protein, and to a method for regulation of cellular interactions using such a fusion protein or a monoclonal antibody.

WO 97/20574 refers to blockade of T lymphocyte down-regulation associated with CTLA-4 signalling, and to a CTLA-4 blocking agent other than an antibody to the extracellular domain of CTLA-4 that increases the response of mammalian T cells to antigenic stimulus or decreases the growth of tumour cells in a mammalian host.

WO 00/37504 refers to human anti-CTLA-4 antibodies and use of such antibodies in treatment of cancer. WO 00/37504 further refers to the human monoclonal antibody tremelimumab, mentioned above, which is denoted 11.2.1 in that patent application.

WO 01/14424 also refers to human antibodies that specifically bind to human CTLA-4, and to use thereof in treatment of human diseases and infections, such as cancer. WO 01/14424 further refers to the human monoclonal antibody ipilimumab, mentioned above and discussed further below, which is denoted 10D1 in that patent application.

### SUMMARY OF THE INVENTION

The present invention relates to antibody molecules that specifically bind to CTLA-4, and have improved depleting effect on CTLA-4 positive cells compared to ipilimumab.

Furthermore, the present invention relates to antibody molecules that specifically bind to CTLA-4, which antibody molecules comprise the 6 CDRs having SEQ ID. NOs: 15, 16, 17, 10, 18 and 19 or the 6 CDRs having SEQ ID. NOs: 22, 23, 24, 10, 25 and 26.

Furthermore, the present invention relates to isolated nucleotide sequences encoding the above antibody molecules.

Furthermore, the present invention relates to plasmids comprising the above nucleotide sequences.

Furthermore, the present invention relates to viruses, such as oncolytic viruses, comprising the above nucleotide sequences or the above plasmids.

Furthermore, the present invention relates to cells, such as CAR T-cells, comprising the above nucleotide sequences or the above plasmids.

Furthermore, the present invention relates to the above antibody molecules, nucleotide sequences, plasmids and/or cells for use in medicine.

Furthermore, the present invention relates to the above antibody molecules, nucleotide sequences, plasmids, viruses and/or cells for use in the treatment of cancer.

Furthermore, the present invention relates to use of the above antibody molecules, nucleotide sequences, plasmids, viruses and/or cells for the manufacture of a pharmaceutical composition for use in the treatment of cancer.

Furthermore, the present invention relates to pharmaceutical compositions comprising at least one of the above antibody molecules, nucleotide sequences, plasmids, viruses and/or cells, and optionally a pharmaceutically-acceptable diluent, carrier or excipient.

Furthermore, the present invention relates to methods for treatment of cancer in a subject comprising administering to the subject a therapeutically effective amount of at least one of the above antibody molecules, nucleotide sequences, plasmids, viruses, cells and/or pharmaceutical compositions.

Furthermore, the present invention relates to an antibody molecule, an antibody molecule for use, an isolated nucleotide sequence, an isolated nucleotide sequence for use, a plasmid, a plasmid for use, a virus, a virus for use, a cell, a cell for use, a use, a pharmaceutical composition or a method of treatment as described herein with reference to the detailed description, examples and/or figures.

### DETAILED DESCRIPTION OF THE INVENTION

CTLA-4 positive cells include regulatory T cells, Treg cells, Tregs or Tregs, (formerly known as suppressor T cells, sometimes also called suppressive regulatory T cells) which is a subpopulation of T cells capable of suppressing other immune cells in normal and pathological immune settings. Tregs are CD4 positive cells (CD4+ cells). There are other CD4+ T cells that are not Tregs; however, Tregs can be separated from non-Treg CD4+ cells in that Tregs also are FOXP3 positive (FOXP3+) while the non-Treg CD4+ cells are FOXP3 negative (FOXP3-).

Like ipilimumab, the anti-CTLA-4 antibody molecules described herein act, at least in part, by depleting CTLA-4 positive cells, such as Tregs. Further, like ipilimumab, the anti-CTLA-4 antibody molecules described herein block CTLA-4 interactions with B7.1 and B7.2. Thus, these antibodies can consequently help overcome CTLA-4 induced suppressive effects on effector T cell proliferation.

By depletion of Tregs, or Treg depletion, we refer herein to depletion, deletion or elimination of Tregs through physical clearance of cells. In particular, we refer to depletion of intratumoural Tregs. Depletion of Tregs may be achieved through ADCC, i.e. antibody-dependent cell-mediated cytotoxicity or antibody-dependent cellular cytotoxicity, and/or ADCP, i.e. antibody dependent cellular phagocytosis. This means that when an antibody molecule as described herein is administered to a subject, such as a human, it binds specifically to CTLA-4 expressed on the surface of Tregs, and this binding results in depletion of the Tregs. In some embodiments, the CTLA-4 is preferentially expressed on tumour infiltrating lymphocytes in the tumour microenvironment or on tumour cells.

ADCC is an immune mechanism through which Fc receptor-bearing effector cells can recognize and kill antibody-coated target cells expressing tumour-derived antigens, i.e. in the present case, CTLA-4, on their surface. ADCP is a similar mechanism, although it results in the target cells being killed through phagocytosis instead of cytotoxicity.

Antibodies are well known to those skilled in the art of immunology and molecular biology. Typically, an antibody comprises two heavy (H) chains and two light (L) chains. Herein, we sometimes refer to this complete antibody molecule as a full-size or full-length antibody. The antibody's heavy chain comprises one variable domain (VH) and three constant domains (CH1, CH2 and CH3), and the antibody's molecule light chain comprises one variable domain (VL) and one constant domain (CL). The variable domains (sometimes collectively referred to as the F_{V} region) bind to the antibody's target, or antigen. Each variable domain comprises three loops, referred to as complementary determining regions (CDRs), which are responsible for target binding. The constant domains are not involved directly in binding an antibody to an antigen, but exhibit various effector functions. Depending on the amino acid sequence of the constant region of their heavy chains, antibodies or immunoglobulins can be assigned to different classes. There are five major classes of immunoglobulins: IgA, IgD, IgE, IgG and IgM, and in humans several of these are further divided into subclasses (isotypes), e.g., IgG1, IgG2, IgG3, and IgG4; IgA1 and IgA2. Another part of an antibody is the Fc domain (otherwise known as the fragment crystallisable domain), which comprises two of the constant domains of each of the antibody's heavy chains. The Fc domain is responsible for interactions between the antibody and Fc receptor.

Fc receptors are membrane proteins which are often found on the cell surface of cells of the immune system (i.e. Fc receptors are found on the target cell membrane-otherwise known as the plasma membrane or cytoplasmic membrane). The role of Fc receptors is to bind antibodies via the Fc domain, and to internalize the antibody into the cell. In the immune system, this can result in antibody-mediated phagocytosis and antibody-dependent cell-mediated cytotoxicity.

The term antibody molecule, as used herein, encompasses full-length or full-size antibodies as well as functional fragments of full-length antibodies and derivatives of such antibody molecules.

Functional fragments of a full-size antibody have the same antigen binding characteristics as the corresponding full-size antibody and include either the same variable domains (i.e. the VH and VL sequences) and/or the same CDR sequences as the corresponding full-size antibody. That the functional fragment has the same antigen binding characteristics as the corresponding full-size antibody means that it binds to the same epitope on the target as the full-size antibody. Such a functional fragment may correspond to the Fv part of a full-size antibody. Alternatively, such a fragment may be a Fab, also denoted F(ab), which is a monovalent antigen-binding fragment that does not contain a Fc part, or a F(ab′)₂, which is an divalent antigen-binding fragment that contains two antigen-binding Fab parts linked together by disulfide bonds or a F(ab′), i.e. a monovalent-variant of a F(ab′)₂. Such a fragment may also be single chain variable fragment (scFv).

A functional fragment does not always contain all six CDRs of a corresponding full-size antibody. It is appreciated that molecules containing three or fewer CDR regions (in some cases, even just a single CDR or a part thereof) are capable of retaining the antigen-binding activity of the antibody from which the CDR(s) are derived. For example, in Gao et al., 1994, J. Biol. Chem., 269:32389-93 it is described that a whole VL chain (including all three CDRs) has a high affinity for its substrate.

Molecules containing two CDR regions have been described, for example, by Vaughan & Sollazzo 2001, Combinatorial Chemistry & High Throughput Screening, 4: 417-430. On page 418 (right column—3 Our Strategy for Design) a minibody including only the H1 and H2 CDR hypervariable regions interspersed within framework regions is described. The minibody is described as being capable of binding to a target. Pessi et al., 1993, Nature, 362: 367-9 and Bianchi et al., 1994, J. Mol. Biol., 236: 649-59 are referenced by Vaughan & Sollazzo and describe the H1 and H2 minibody and its properties in more detail. In Qiu et al., 2007, Nature Biotechnology, 25:921-9 it is demonstrated that a molecule consisting of two linked CDRs are capable of binding antigen. Quiocho 1993, Nature, 362:293-4 provides a summary of “minibody” technology. Ladner 2007, Nature Biotechnology, 25:875-7 comments that molecules containing two CDRs are capable of retaining antigen-binding activity.

Antibody molecules containing a single CDR region are described, for example, in Laune et al., 1997, JBC, 272:30937-44, in which it is demonstrated that a range of hexapeptides derived from a CDR display antigen-binding activity and it is noted that synthetic peptides of a complete, single, CDR display strong binding activity. In Monnet et al., 1999, JBC, 274:3789-96 it is shown that a range of 12-mer peptides and associated framework regions have antigen-binding activity and it is commented on that a CDR3-like peptide alone is capable of binding antigen. In Heap et al., 2005, J. Gen. Virol., 86:1791-1800 it is reported that a “micro-antibody” (a molecule containing a single CDR) is capable of binding antigen and it is shown that a cyclic peptide from an anti-HIV antibody has antigen-binding activity and function. In Nicaise et al., 2004, Protein Science, 13:1882-91 it is shown that a single CDR can confer antigen-binding activity and affinity for its lysozyme antigen.

Thus, antibody molecules having five, four, three or fewer CDRs are capable of retaining the antigen binding properties of the full-length antibodies from which they are derived.

The antibody molecule may also be a derivative of a full-length antibody or a fragment of such an antibody. The derivative has the same antigen binding characteristics as the corresponding full-size antibody in the sense that it binds to the same epitope on the target as the full-size antibody.

Thus, by the term “antibody molecule”, as used herein, we include all types of antibody molecules and functional fragments thereof and derivatives thereof, including: monoclonal antibodies, polyclonal antibodies, synthetic antibodies, recombinantly produced antibodies, multi-specific antibodies, bi-specific antibodies, human antibodies, humanized antibodies, chimeric antibodies, single chain antibodies, variable fragments (Fvs), single-chain variable fragments (scFv fragments) including divalent single-chain variable fragments (di-scFvs) and disulfide-linked variable fragments, Fab fragments, F(ab′)₂ fragments, Fab′ fragments, antibody heavy chains, antibody light chains, homo-dimers of antibody heavy chains, homo-dimers of antibody light chains, heterodimers of antibody heavy chains, heterodimers of antibody light chains, antigen binding functional fragments of such homo- and heterodimers.

Further, the term “antibody molecule”, as used herein, includes all classes of antibody molecules and functional fragments, including: IgG, IgG1, IgG2, IgG3, IgG4, IgA, IgM, IgD, and IgE.

In some embodiments, the antibody is a human IgG1. The skilled person is aware that the mouse IgG2a and human IgG1 productively engage with activatory Fc gamma receptors and share the ability to activate deletion of target cells through activation of activatory Fc gamma receptor bearing immune cells (e.g. macrophages and NK cells) by e.g. ADCP and ADCC. As such, whereas the mouse IgG2a is the preferred isotype for deletion in the mouse, human IgG1 is a preferred isotype for deletion in human. Conversely, it is known that optimal co-stimulation of TNFR superfamily agonist receptors e.g. 4-1BB, OX40, TNFRII, CD40 depends on antibody engagement of the inhibitory FcγRII. In the mouse the IgG1 isotype, which binds preferentially to inhibitory Fc gamma receptor (FcγRIIB) and only weakly to activatory Fc gamma receptors, is known to be optimal for costimulatory activity of TNFR-superfamily targeting mAb. While no direct equivalent of the mouse IgG1 isotype has been described in man, antibodies may be engineered to show a similarly enhanced binding to inhibitory over activatory human Fc gamma receptors. Such engineered TNFR-superfamily targeting antibodies also have improved co-stimulatory activity in vivo, in transgenic mice engineered to express human activatory and inhibitory Fc gamma receptors (Dahan et al, 2016, Therapeutic Activity of Agonistic, Human Anti-CD40 Monoclonal Antibodies Requires Selective FcγR Engagement. Cancer Cell. 29(6):820-31).

As outlined above, different types and forms of antibody molecules are included in the invention, and would be known to the person skilled in immunology. It is well known that antibodies used for therapeutic purposes are often modified with additional components which modify the properties of the antibody molecule.

Accordingly, we include that an antibody molecule of the invention or an antibody molecule used in accordance with the invention (for example, a monoclonal antibody molecule, and/or polyclonal antibody molecule, and/or bi-specific antibody molecule) comprises a detectable moiety and/or a cytotoxic moiety.

By “detectable moiety”, we include one or more from the group comprising of: an enzyme; a radioactive atom; a fluorescent moiety; a chemiluminescent moiety; a bioluminescent moiety. The detectable moiety allows the antibody molecule to be visualised in vitro, and/or in vivo, and/or ex vivo.

By “cytotoxic moiety”, we include a radioactive moiety, and/or enzyme, for example wherein the enzyme is a caspase, and/or toxin, for example wherein the toxin is a bacterial toxin or a venom; wherein the cytotoxic moiety is capable of inducing cell lysis.

We further include that the antibody molecule may be in an isolated form and/or purified form, and/or may be PEGylated.

As discussed above, the CDRs of an antibody bind to the antibody target. The assignment of amino acids to each CDR described herein is in accordance with the definitions according to Kabat E A et al. 1991, In “Sequences of Proteins of Immulogical Interest” Fifth Edition, NIH Publication No. 91-3242, pp xv-xvii.

As the skilled person would be aware, other methods also exist for assigning amino acids to each CDR. For example, the International ImMunoGeneTics information system (IMGT(R)) (world-wide-web at imgt.org and Lefranc and Lefranc “The Immunoglobulin FactsBook” published by Academic Press, 2001).

In a further embodiment, the antibody molecule of the present invention or used according to the invention is an antibody molecule that is capable of competing with the specific antibodies described herein, such as the antibody molecules comprising SEQ ID. NOs: 15, 16, 17, 10, 18 and 19 or SEQ ID. NOs: 22, 23, 24, 10, 25 and 26.

By “capable of competing for” we mean that the competing antibody is capable of inhibiting or otherwise interfering, at least in part, with the binding of an antibody molecule as defined herein to the specific target.

For example, such a competing antibody molecule may be capable of inhibiting the binding of an antibody molecule described herein by at least about 10%; for example at least about 20%, or at least about 30%, at least about 40%, at least about 50%, at least about 60%, at least about 70%, at least about 80%, at least about 90%, at least about 95%, at least about 100% and/or inhibiting the ability of the antibody described herein to prevent or reduce binding to the specific target by at least about 10%; for example at least about 20%, at least about 30%, at least about 40%, at least about 50%, at least about 60%, at least about 70%, at least about 80%, at least about 90%, at least about 95%, or at least about 100%.

Competitive binding may be determined by methods well known to those skilled in the art, such as Enzyme-linked immunosorbent assay (ELISA).

ELISA assays can be used to evaluate epitope-modifying or blocking antibodies. Additional methods suitable for identifying competing antibodies are disclosed in Antibodies: A Laboratory Manual, Harlow & Lane, which is incorporated herein by reference (for example, see pages 567 to 569, 574 to 576, 583 and 590 to 612, 1988, CSHL, NY, ISBN 0-87969-314-2).

It is well known that an antibody specifically binds a defined target molecule or antigen, and that this means that the antibody preferentially and selectively binds its target and not a molecule which is not a target.

The target CTLA-4 of the antibodies according to the present invention, or of the antibodies used in accordance with the invention, are expressed on the surface of cells, i.e. they are cell surface antigen, which would include an epitope (otherwise known in this context as a cell surface epitope) for the antibody. Cell surface antigen and epitope are terms that would be readily understood by one skilled in immunology or cell biology.

By “cell surface antigen”, we include that the cell surface antigen or at least the epitope thereof to which the antibody molecule described herein, is exposed on the extracellular side of the cell membrane.

Methods of assessing protein binding are known to the person skilled in biochemistry and immunology. It would be appreciated by the skilled person that those methods could be used to assess binding of an antibody to a target and/or binding of the Fc domain of an antibody to an Fc receptor; as well as the relative strength, or the specificity, or the inhibition, or prevention, or reduction in those interactions. Examples of methods that may be used to assess protein binding are, for example, immunoassays, BIAcore, western blots, radioimmunoassay (RIA) and enzyme-linked immunosorbent assays (ELISAs) (See Fundamental Immunology Second Edition, Raven Press, New York at pages 332-336 (1989) for a discussion regarding antibody specificity).

Accordingly, herein both an “antibody molecule that specifically binds CTLA-4” and an “anti-CTLA-4 antibody molecule” refers to an antibody molecule that specifically binds the target CTLA-4 but does not bind to non-target, or binds to a non-target more weakly (such as with a lower affinity) than the target.

In some embodiments, the antibody molecule that specifically binds CTLA-4 (or the anti-CTLA-4 antibody molecule) refers to an antibody molecule that specifically binds to the extracellular domain of CTLA-4.

In some embodiments, the antibody molecule that specifically binds CTLA-4 (or the anti-CTLA-4 antibody molecule) does not cross react with CD28. In some embodiments, the antibody molecule that specifically binds CTLA-4 (or the anti-CTLA-4 antibody molecule) blocks the binding of CTLA-4 to CD80 and/or CD86, thereby inhibiting CLTA-4 signalling.

We also include the meaning that the antibody specifically binds to the target CTLA-4 at least two-fold more strongly, or at least five-fold more strongly, or at least 10-fold more strongly, or at least 20-fold more strongly, or at least 50-fold more strongly, or at least 100-fold more strongly, or at least 200-fold more strongly, or at least 500-fold more strongly, or at least than about 1000-fold more strongly than to a non-target.

Additionally, we include the meaning that the antibody specifically binds to the target CTLA-4 if it binds to the target with a K_{d} of at least about 10⁻¹ K_{d}, or at least about 10⁻² K_{d}, or at least about 10⁻³ K_{d}, or at least about 10⁻⁴ K_{d}, or at least about 10⁻⁵ K_{d}, or at least about 10⁻⁶ K_{d}, or at least about 10⁻⁷ K_{d}, or at least about 10⁻⁸ K_{d}, or at least about 10⁻⁹ K_{d}, or at least about 10⁻¹⁰ K_{d}, or at least about 10⁻¹¹ K_{d}, or at least about 10⁻¹² K_{d}, or at least about 10⁻¹³ K_{d}, or at least about 10⁻¹⁴ K_{d}, or at least about 10⁻¹⁵ K_{d}.

As mentioned above, the antibody molecules that specifically bind to CTLA-4 (or the anti-CTLA-4 antibody molecules) described herein has an improved depleting effect on CTLA-4 positive cells compared to ipilimumab.

That the antibody molecules have a depleting effect on CTLA-4 positive cells means that upon administration to a subject, such as a human, such an antibody binds specifically to CTLA-4 expressed on the surface of CTLA-4 positive cells, and this binding results in depletion of such cells.

In some embodiments, the CTLA-4 positive cells are CD4 positive (CD4+) cells, i.e. cells that express CD4.

In some embodiments, the CTLA-4 positive cells are both CD4 positive and FOXP3 positive, i.e. expressing both CD4 and FOXP3. These cells are Tregs. CD8 positive T cells also express CTLA-4, but Tregs express significantly higher levels of CTLA-4 than CD8 positive T cells. This makes Tregs more susceptible to depletion compared to lower expressing CD8+ cells.

In some situations, the CTLA-4 is preferentially expressed on immune cells in the tumour microenvironment (tumour infiltrating cells, TILS).

Thus, in a tumour microenvironment, the Tregs will be the cells that have the highest expression of CTLA-4, resulting in the antibody molecules that specifically bind to CTLA-4 (or the anti-CTLA-4 antibody molecules) having a Treg depleting effect. This is discussed in more detail below, e-g. in Example 4 and in connection with FIG. 13.

In some embodiments, CTLA-4 positive cells will be Tregs in a solid tumour. Such Tregs will have very high expression of CTLA-4, and therefore administration of an antibody molecules that specifically binds to CTLA-4, will preferentially result in depletion of such Tregs.

As mentioned above, the anti-CTLA-4 antibody molecules described herein are Treg depleting antibody molecules, which means that upon administration to a subject, such as a human, such an antibody molecule binds specifically to CTLA-4 expressed on the surface of Tregs, and this binding results in depletion of Tregs.

To decide whether an antibody molecule is an antibody molecule that has improved depleting effect on CTLA-4 positive cells compared to ipilimumab as referred to herein, it is possible to use an in vitro antibody-dependent cellular cytotoxicity (ADCC) assay or an in vivo test in a PBMC-NOG/SCID model.

The in vitro ADCC test which is performed using an NK-92 cell line stably transfected to express the CD16-158V allele together with GFP, wherein the ADCC test comprises the following consecutive seven steps:
1) CTLA-4 positive cells, CD4 positive cells or Tregs as target cells are isolated from peripheral blood of healthy donors. This isolation may be done using a CD4+ T cell isolation kit, such as a commercial kit from Miltenyi Biotec.
2) The target cells are then stimulated, e.g. for 48 hours, with CD3/CD28, for example using CD3/CD28 Dynabeads® and rhIL-2, such as 50 ng/ml rhIL-2. The stimulation may be done at 37° C.
3) The target cells are then pre-incubated with the antibody molecule to be tested, e.g. at 10 μg/ml for 30 min at 4° C., and are then mixed with NK cells. 4) The target cells are then incubated for an appropriate time, such as 4 hours, in RPMI 1640+GlutaMAX medium containing HEPES buffer, sodium pyruvate and FBS low IgG. The RPMI 1640+GlutaMAX medium may containing 10 mM HEPES buffer, 1 mM sodium pyruvate and 10% FBS low IgG, and the effector:target cell ratio may be 2:1.
5) Lysis is determined by flow cytometry.
6) Steps 1-5 are repeated, or performed in parallel, with ipilimumab used instead of the tested antibody molecule in step 3.
7) The results of the lysis for the tested antibody molecule are compared to the results of the lysis for ipilimumab. An improved lysis for the tested antibody molecule compared to ipilimumab demonstrates that the tested antibody molecule has improved depleting effect on CTLA-4 positive cells, CD4 positive cells or Tregs, respectively, depending on which target cells were used.

In some embodiments, the improved depleting effect in step 7) above is a significantly improved depleting effect.

This assay is demonstrated in more detail below in Example 4, in combination with FIG. 12.

The in vivo test is based on the combined use of PBMC mice and NOG/SCID mice, which is herein called a PBMC-NOG/SCID model. Both PBMC mice and NOG/SCID mice are well-known models. The in vivo test in the PBMC-NOG/SCID model comprises the following consecutive nine steps:
1) Human PBMCs (peripheral blood mononuclear cells) are isolated, washed and resuspended in sterile PBS. In some embodiments, the PBMCs are resuspended in PBS at 75×10⁶ cells/ml.
2) NOG mice are injected i.v. (intravenously) with an appropriate amount, such as 200 μl, of the cell suspension from step 1). If 200 μl are injected, this corresponds to 15×10⁶ cells/mouse.
3) A suitable time, such as 2 weeks, after injection, the spleens from the NOG mice are isolated and rendered into a single cell suspension. Optionally, a small sample from the single cell suspension is taken to determine the expression of CTLA-4 on human T cells by FACS, in order to confirm the CTLA-4 expression.
4) The cell suspension from step 3) is resuspended in sterile PBS. In some embodiments, the cell suspension is resuspended in sterile PBS at 50×10⁶ cells/ml. If the optional CTLA-4 expression determination is included in step 3, the rest of the cell suspension is then resuspended in step 4.
5) SCID mice are injected i.p. (intraperitoneally) with an appropriate amount, such as 200 μl, of the suspension from step 4. If 200 μl are injected, this corresponds to 10×10⁶ cells/mouse.
6) A suitable time, such as 1 hour, after the injection in step 5) the SCID mice are treated with an appropriate amount, such as 10 mg/kg, of either the antibody molecule to be tested, ipilimumab or an isotype control monoclonal antibody.
7) The intraperitoneal fluid of the treated SCID mice is collected a suitable time, such as 24 hours, after the treatment in step 6).
8) Human T cell subsets are identified and quantified by FACS using following markers: CD45, CD4, CD8, CD25 and/or CD127.
9) The results from identification and quantification of the T cell subsets from the mice treated with the tested antibody molecule is compared to the results from identification and quantification of the T cell subsets from the mice treated with ipilimumab and to the results from identification and quantification of the T cell subsets from the mice treated with isotype control monoclonal antibody. A lower number of CTLA-4 positive cells in the intraperitoneal fluid from mice treated with the antibody molecule to be tested compared to the number of CTLA-4 positive cells in the intraperitoneal fluid from mice treated with ipilimumab demonstrates that the antibody molecule has improved depleting effect on CTLA-4 positive cells compared to ipilimumab. A lower number of CD4 positive cells in the intraperitoneal fluid from mice treated with the antibody molecule to be tested compared to the number of CD4 positive cells in the intraperitoneal fluid from mice treated with ipilimumab demonstrates that the antibody molecule has improved depleting effect on CD4 positive cells compared to ipilimumab. A lower number of Tregs in the intraperitoneal fluid from mice treated with the antibody molecule to be tested compared to the number of Tregs in the intraperitoneal fluid from mice treated with ipilimumab demonstrates that the antibody molecule has improved depleting effect on Tregs compared to ipilimumab.

In this in vivo test, it is in some embodiments of most interest to look at the Treg depletion in step 7.

This assay is demonstrated in more detail below in Example 4, in combination with FIG. 14.

Treg depletion may also be assessed in an antibody-dependent cellular phagocytosis (ADCP) assay, as known to the skilled person.

In some embodiments, the antibody molecules have similar blocking effect on CTLA-4 interactions with B7.1 and B7.2 ligands compared to Yervoy. This may be assessed by ELISA (as shown in FIG. 10) or in a more functional assay where anti-CTLA-4 antibodies enhance the IL-2 production by T cells in response to stimulation of PBMCs with SEB.

In some embodiments, the anti-CTLA-4 antibody molecule is a human antibody molecule.

In some embodiments, the anti-CTLA-4 antibody molecule is a humanized antibody molecule.

In some embodiments, the anti-CTLA-4 antibody molecule is an antibody molecule of human origin, meaning that it originates from a human antibody molecule which then has been modified.

In some embodiments, the anti-CTLA-4 antibody molecule is a human IgG1 antibody.

In some embodiments, the anti-CTLA-4 antibody is an antibody in the form of a human IgG1 antibody showing improved binding to one or several activatory Fc receptors and/or being engineered for improved binding to one or several activatory Fc receptors; accordingly, in some embodiments, the anti-CTLA-4 antibody is an Fc-engineered human IgG1 antibody.

In some embodiments, the anti-CTLA-4 antibody is a murine or a humanized murine IgG2a antibody.

In some embodiments, the anti-CTLA-4 antibody is a murine antibody that is cross-reactive with human CTLA-4.

In some embodiments, the anti-CTLA-4 antibody is a monoclonal antibody.

In some embodiments, the anti-CTLA-4 antibody is a polyclonal antibody.

In some embodiments, the anti-CTLA-4 antibody molecule is an antibody molecule comprising one of the three alternative VH-CDR1 sequences, one of the three alternative VH-CDR2 sequences, one of the two alternative VH-CDR3 sequences, one of the two VL-CDR1 sequences, one of the two VL-CDR2 sequences, and/or one of the two alternative VL-CDR3 sequences presented in Table 1 below.

In some embodiments, the anti-CTLA-4 antibody molecule is selected from the group consisting of antibody molecules comprising 1-6 of the CDRs selected from the group consisting of SEQ ID. Nos: 3, 6, 8, 10, 12 and 14.

In some embodiments, the anti-CTLA-4 antibody molecule is selected from the group consisting of antibody molecules comprising the CDRs having SEQ ID. Nos: 3, 6, 8, 10, 12 and 14.

In some embodiments, the anti-CTLA-4 antibody molecule is selected from the group consisting of antibody molecules comprising 1-6 of the CDRs, VH-CDR1, VH-CDR2, VH-CDR3, VL-CDR1, and VL-CDR3,
wherein VH-CDR1, if present, is selected from the group consisting of SEQ ID. Nos: 15, 22, 29 and 35;
wherein VH-CDR2, if present, is selected from the group consisting of SEQ ID. Nos: 16, 23, 30, and 36;
wherein VH-CDR3, if present, is selected from the group consisting of SEQ ID. Nos: 17, 24, 31 and 37;
wherein VL-CDR1, if present, is selected from the group consisting of SEQ ID. Nos: 10 and 38;
wherein VL-CDR2, if present, is selected from the group consisting of SEQ ID. Nos: 18, 25, 32 and 39;
wherein VL-CDR3, if present, is selected from the group consisting of SEQ ID. Nos: 19, 26 and 40.

In some embodiments, the anti-CTLA-4 antibody molecule is an antibody molecule selected from the group consisting of antibody molecules comprising 6 CDRs selected from the group consisting of:
SEQ ID. NOs: 15, 16, 17, 10, 18 and 19;
SEQ ID. NOs: 22, 23, 24, 10, 25 and 26;
SEQ ID. NOs: 29, 30, 31, 10, 32 and 26; and
SEQ ID. NOs: 35, 36, 37, 38, 39 and 40.

In some embodiments, the anti-CTLA-4 antibody molecule is an antibody molecule comprising the 6 CDRs having SEQ ID. NOs: 15, 16, 17, 10, 18 and 19.

In some embodiments, the anti-CTLA-4 antibody molecule is an antibody molecule comprising the 6 CDRs having SEQ ID. NOs: 22, 23, 24, 10, 25 and 26.

In some embodiments, the anti-CTLA-4 antibody molecule is an antibody molecule selected from the group consisting of antibody molecules comprising a VH selected from the group consisting of SEQ ID. NOs: 20, 27, 33 and 41.

In some embodiments, the anti-CTLA-4 antibody molecule is an antibody molecule selected from the group consisting of antibody molecules comprising a VL selected from the group consisting of SEQ ID. NOs: 21, 28, 34 and 42.

In some embodiments, the anti-CTLA-4 antibody molecule is an antibody molecule selected from the group consisting of antibody molecules comprising a VH and a VL selected from the group consisting of: SEQ ID. Nos: 20-21, 27-28, 33-34 and 41-42.

In some embodiments, the anti-CTLA-4 antibody molecule comprises a VH having sequence SEQ ID. No: 20 and a VL having sequence SEQ ID. No: 21.

In some embodiments, the anti-CTLA-4 antibody molecule comprises a VH having sequence SEQ ID. No: 27 and a VL having sequence SEQ ID. No: 28.

TABLE 1
General CDR sequences of antibodies disclosed herein

| | | Explanation | |
|---|---|---|---|
| Relevant part | | of unidentified | SEQ. |
| of antibody | Sequence | amino acid residues | ID. NO |
| VH-CDR1 | FX₁X₂YX₃MX₄WX₅R QAPG | X₁ = S or K; | 1 |
| alternative 1 | | X₂ = D, S or A; | |
| | | X₃ = Y, S or A; | |
| | | X₄ = S or N; and | |
| | | X₅ = V or I | |
| VH-CDR1 | FSX₁YX₂MX₃WVRQ APG | X₁ = D or S; | 2 |
| alternative 2 | | X₂ = Y, S or A; and | |
| | | X₃ = S or N | |
| VH-CDR3 | FSX₁YX₂MX₃WVRQ APG | X₁ = D or S; | 3 |
| alternative 3 | | X₂ = Y or S; and | |
| | | X₃ = S or N | |
| VH-CDR2, | SX₁ISX₂X₃X₄X₅X₆X₇X₈X₉ADSVKGR | X₁ = G or A; | 4 |
| alternative 1 | | X₂ = W, G or N; | |
| | | X₃ = S or T; | |
| | | X₄ = S or G; | |
| | | X₅ = R or G; | |
| | | X₆ = D, S or Y; | |
| | | X₇ = K, T or I; | |
| | | X₈ = G, Y, H or D; | |
| | | X₉ = Y or F | |
| VH-CDR2, | SX₁ISX₂X₃X₄X₅X₆X₇X₈YADSVKGR | X₁ = G or A; | 5 |
| alternative 2 | | X₂ = T, G or N; | |
| | | X₃ = S or T; | |
| | | X₄ = S or G; | |
| | | X₅ = R or G; | |
| | | X₆ = D, S or Y; | |
| | | X₇ = K, T or I; | |
| | | X₈ = G, Y, H or D | |
| VH-CDR2, | SX₁ISX₂X₃X₄X₅X₆X₇ YADSVKGR | X₁ = G or A; | 6 |
| alternative 3 | | X₂ = W or G; | |
| | | X₃ = S or G; | |
| | | X₄ = R or G; | |
| | | X₅ = D or S; | |
| | | X₆ = K or T; | |
| | | X₇ = G or Y, H or D | |
| VH-CDR3 | X₁X₂X₃X₄X₅X₆X₇X₈X₉X₁₀X₁₁X₁₂X₁₃X₁₄X₁₅ | X₁ = T or A; | 7 |
| alternative 1 | | X₂ = T or R; | |
| | | X₃ = D, Y or L; | |
| | | X₄ = L, R, S, or G; | |
| | | X₅ = A, V, S or Y; | |
| | | X₆ = R, E, G or S; | |
| | | X₇ = Y, M, L or G; | |
| | | X₈ = N, H, Y or none; | |
| | | X₉ = Q, D or none; | |
| | | X₁₀ = W, A, D or none; | |
| | | X₁₁ = L, F, R or none; | |
| | | X₁₂ = A, D, G or none; | |
| | | X₁₃ = D, I, M or none; | |
| | | X₁₄ = D or none; and | |
| | | X₁₅ = V or none | |
| VH-CDR3 | X₁X₂DX₃X₄X₅X₆X₇X₈X₉X₁₀X₁₁X₁₂X₁₃ | X₁ = T or A; | 8 |
| alternative 2 | | X₂ = T or R;; | |
| | | X₃ = L or R; | |
| | | X₄ = A or V; | |
| | | X₅ = R or E; | |
| | | X₆ = Y or M; | |
| | | X₇ = N or none; | |
| | | X₈ = Q or none; | |
| | | X₉ = W or none; | |
| | | X₁₀ = L, or none; | |
| | | X₁₁ = A, or none; | |
| | | X₁₂ = D or none | |
| VL-CDR1 | CX₁GSSSNIGX₂X₃YX₄X₅X₆ | X₁ = T or S; | 9 |
| alternative 1 | | X₂ = A or S; | |
| | | X₃ = G or N; | |
| | | X₄ = D or V; | |
| | | X₅ = V or Y; | |
| | | X₆ = H or none | |
| VL-CDR1 | CTGSSSNIGAGYDVH | | 10 |
| alternative 2 | | | |
| VL-CDR2 | X₁NX₂X₃RPS | X₁ = G, R, or D; | 11 |
| alternative 1 | | X₂ = D, N, or S; and | |
| | | X₃ = N, Q or K | |
| VL-CDR2 | X₁NX₂X₃RPS | X₁ = G or R; | 12 |
| alternative 2 | | X₂ = D or N; and | |
| | | X₃ = N or Q | |
| VL-CDR3 | CX₁X₂X₃DX₄SLX₅G X₆VX₇ | X₁ = A or Q; | 13 |
| alternative 1 | | X₂ = V, A or S; | |
| | | X₃ = W or Y; | |
| | | X₄ = D or S; | |
| | | X₅ = N or S; | |
| | | X₆ = V, W or P; and | |
| | | X₇ = V or none | |
| VL-CDR3 | CAX₁WDDSLNG X₂V | X₁ = V or A; and | 14 |
| alternative 2 | | X₂ = V or W | |

TABLE 2
Specific anti-CTLA-4 antibody molecules; the CDR sequences
are marked in bold in the full VH and VL sequences

| Antibody | | | SEQ. |
|---|---|---|---|
| clone | Region | Sequence | ID. NO: |
| 4-E03 | VH-CDR1 | FSDYYMSWVR QAPG | 15 |
| | VH-CDR2 | SGISWSSRDK GYADSVKGR | 16 |
| | VH-CDR3 | TTDLARY | 17 |
| | VL-CDR1 | CTGSSSNIGA GYDVH | 10 |
| | VL-CDR2 | GNDNRPS | 18 |
| | VL-CDR3 | CAVWDDSLNG VV | 19 |
| | VH | EVQLLESGGG LVQPGGSLRL SCAASGFTFS DYYMSWVRQA | 20 |
| | | PGKGLEWVSG ISWSSRDKGY ADSVKGRFTI SRDNSKNTLY | |
| | | LQMNSLRAED TAVYYCTTDL ARYWGQGTLV TVSS | |
| | VL | QSVLTQPPSA SGTPGQRVTI SCTGSSSNIG AGYDVHWYQQ | 21 |
| | | LPGTAPKLLI YGNDNRPSGV PDRFSGSKSG TSASLAISGL | |
| | | RSEDEADYYC AVWDDSLNGV VFGGGTKLTV LG | |
| | human | MGWSCIILFLVATATGVHSQSVLTQPPSASGTPGQRVTISCTG | 53 |
| | immune_globulin | SSSNIGAGYDVHWYQQLPGTAPKLLIYGNDNRPSGVPDRFSGS | |
| | G1 (IgG1) light | KSGTSASLAISGLRSEDEADYYCAVWDDSLNGVVFGGGTKLTV | |
| | chain (LC) | LGQPKAAPSVTLFPPSSEELQANKATLVCLISDFYPGAVTVAW | |
| | | KADSSPVKAGVETTTPSKQSNNKYAASSYLSLTPEQWKSHRSY | |
| | | SCQVTHEGSTVEKTVAPTECS | |
| | human IgG1 | MGWSCIILFLVATATGVHSEVQLLESGGGLVQPGGSLRLSCAA | 54 |
| | heavy chain | SGFTFSDYYMSWVRQAPGKGLEWVSGISWSSRDKGYADSVKGR | |
| | (HC) | FTISRDNSKNTLYLQMNSLRAEDTAVYYCTTDLARYWGQGTLV | |
| | | TVSSASTKGPSVFPLAPSSKSTSGGTAALGCLVKDYFPEPVTV | |
| | | SWNSGALTSGVHTFPAVLQSSGLYSLSSVVTVPSSSLGTQTYI | |
| | | CNVNHKPSNTKVDKKVEPKSCDKTHTCPPCPAPELLGGPSVFL | |
| | | FPPKPKDTLMISRTPEVTCVVVDVSHEDPEVKFNWYVDGVEVH | |
| | | NAKTKPREEQYNSTYRVVSVLTVLHQDWLNGKEYKCKVSNKAL | |
| | | PAPIEKTISKAKGQPREPQVYTLPPSRDELTKNQVSLTCLVKG | |
| | | FYPSDIAVEWESNGQPENNYKTTPPVLDSDGSFFLYSKLTVDK | |
| | | SRWQQGNVFSCSVMHEALHNHYTQKSLSLSPGK | |
| 5-B07 | VH-CDR1 | FSSYSMNWVRQ APG | 22 |
| | VH-CDR2 | SAISGSGGST YYADSVKGR | 23 |
| | VH-CDR3 | ARDRVEMNQW LAD | 24 |
| | VL-CDR1 | CTGSSSNIGA GYDVH | 10 |
| | VL-CDR2 | RNNQRPS | 25 |
| | VL-CDR3 | CAAWDDSLNG WV | 26 |
| | VH | EVQLLESGGG LVQPGGSLRL SCAASGFTFS SYSMNWVRQA | 27 |
| | | PGKGLEWVSA ISGSGGSTYY ADSVKGRFTI SRDNSKNTLY | |
| | | LQMNSLRAED TAVYYCARDR VEMNQWLADW GQGTLVTVSS | |
| | VL | QSVLTQPPSA SGTPGQRVTI SCTGSSSNIG AGYDVHWYQQ | 28 |
| | | LPGTAPKLLI YRNNQRPSGV PDRFSGSKSG TSASLAISGL | |
| | | RSEDEADYYC AAWDDSLNGW VFGGGTKLTV LG | |
| | murine | MGWSCIILFLVATATGVHSQSVLTQPPSASGTPGQRVTISCTG | 62 |
| | immunoglobulin | SSSNIGAGYDVHWYQQLPGTAPKLLIYRNNQRPSGVPDRFSGS | |
| | (IgG2a) light | KSGTSASLAISGLRSEDEADYYCAAWDDSLNGWVFGGGTKLTV | |
| | chain (LC) | LGQPKSSPSVTLFPPSSEELETNKATLVCTITDFYPGVVTVDW | |
| | | KVDGTPVTQGMETTQPSKQSNNKYMASSYLTLTARAWERHSSY | |
| | | SCQVTHEGHTVEKSLSRADCS | |
| | murine | MGWSCIILFLVATATGVHSEVQLLESGGGLVQPGGSLRLSCAA | 63 |
| | immunoglobulin | SGFTFSSYSMNWVRQAPGKGLEWVSAISGSGGSTYYADSVKGR | |
| | (IgG2a) heavy | FTISRDNSKNTLYLQMNSLRAEDTAVYYCARDRVEMNQWLADW | |
| | chain (HC) | GQGTLVTVSSAKTTAPSVYPLAPVCGDTTGSSVTLGCLVKGYF | |
| | | PEPVTLTWNSGSLSSGVHTFPAVLQSDLYTLSSSVTVTSSTWP | |
| | | SQSITCNVAHPASSTKVDKKIEPRGPTIKPCPPCKCPAPNLLG | |
| | | GPSVFIFPPKIKDVLMISLSPIVTCVVVDVSEDDPDVQISWFV | |
| | | NNVEVHTAQTQTHREDYNSTLRVVSALPIQHQDWMSGKEFKCK | |
| | | VNNKDLPAPIERTISKPKGSVRAPQVYVLPPPEEEMTKKQVTL | |
| | | TCMVTDFMPEDIYVEWTNNGKTELNYKNTEPVLDSDGSYFMYS | |
| | | KLRVEKKNWVERNSYSCSVVHEGLHNHHTTKSFSRTPGK | |
| 2-C06 | VH-CDR1 | FSSYAMSWVRQ APG | 29 |
| | VH-CDR2 | SGISGSGGYI HYADSVKGR | 30 |
| | VH-CDR3 | ATYSSGLHDA FDI | 31 |
| | VL-CDR1 | CTGSSSNIGA GYDVH | 10 |
| | VL-CDR2 | DNNKRPS | 32 |
| | VL-CDR3 | CAAWDDSLNG WV | 26 |
| | VH | EVQLLESGGG LVQPGGSLRL SCAASGFTFS SYAMSWVRQA | 33 |
| | | PGKGLEWVSG ISGSGGYIHY ADSVKGRFTI SRDNSKNTLY | |
| | | LQMNSLRAED TAVYYCATYS SGLHDAFDIW GQGTLVTVSS | |
| | VL | QSVLTQPPSA SGTPGQRVTI SCTGSSSNIG AGYDVHWYQQ | 34 |
| | | LPGTAPKLLI YDNNKRPSGV PDRFSGSKSG TSASLAISGL | |
| | | RSEDEADYYC AAWDDSLNGW VFGGGTKLTV LG | |
| 2-F09 | VH-CDR1 | FKAYSMSWIR QAPG | 35 |
| | VH-CDR2 | SGISNTGGST DFADSVKGR | 36 |
| | VH-CDR3 | ARLGYSGYDD RGMDV | 37 |
| | VL-CDR1 | CSGSSSNIGS NYVY | 38 |
| | VL-CDR2 | GNSNRPS | 39 |
| | VL-CDR3 | CQSYDSSLSG PVV | 40 |
| | VH | EVQLLESGGG LVQPGGSLRL SCAASGFTFK AYSMSWIRQA | 41 |
| | | PGKGLEWVSG ISNTGGSTDF ADSVKGRPTI SRDNSKNTLY | |
| | | LQMNSLRAED TAMYYCARLG YSGYDDRGMD VWGQGTLVTV | |
| | | SS | |
| | VL | QSVLTQPPSA SGTPGQRVTI SCSGSSSNIG SNYVYWYQQL | 42 |
| | | PGTAPKLLIY GNSNRPSGVP DRFSGSKSGT SASLAISGLR | |
| | | SEDEADYYCQ SYDSSLSGPV VFGGGTKLTV LG | |

In some embodiments, the anti-CTLA-4 antibody molecules described herein may also comprise one or both of the constant regions presented in Table 3 below.

TABLE 3
Sequences of constant regions of
antibodies disclosed herein

| | | SEQ. |
|---|---|---|
| Region | Sequence | ID. NO: |
| CH | ASTKGPSVFPLAPSSKSTSGGTAALGCLVKDYF | 43 |
| | PEPVTVSWNSGALTSGVHTFPAVLQSSGLYSLS | |
| | SVVTVPSSSLGTQTYICNVNHKPSNTKVDKKVE | |
| | PKSCDKTHTCPPCPAPELLGGPSVFLFPPKPKD | |
| | TLMISRTPEVTCVVVDVSHEDPEVKFNWYVDGV | |
| | EVHNAKTKPREEQYNSTYRVVSVLTVLHQDWLN | |
| | GKEYKCKVSNKALPAPIEKTISKAKGQPREPQV | |
| | YTLPPSRDELTKNQVSLTCLVKGFYPSDIAVEW | |
| | ESNGQPENNYKTTPPVLDSDGSFFLYSKLTVDK | |
| | SRWQQGNVFSCSVMHEALHNHYTQKSLSLSPGK | |
| CL | QPKAAPSVTLFPPSSEELQANKATLVCLISDFY | 44 |
| | PGAVTVAWKADSSPVKAGVETTTPSKQSNNKYA | |
| | ASSYLSLTPEQWKSHRSYSCQVTHEGSTVEKTV | |
| | APTECS | |

In some embodiments, the anti-CTLA-4 antibody molecule is a molecule encoded by one of the nucleotide sequences presented in Table 4 below.

TABLE 4
Specific nucleotide sequences
encoding anti-CTLA-4 antibody molecules

| | | | SEQ. |
|---|---|---|---|
| Clone | Encoding | Sequence | ID. NO: |
| 4-E03 γ1 | 4-E03 | GAGGTGCAGCTGTTGGAGTCTGGGGGAGGCTTGGTACAG | 45 |
| heavy chain | VH | CCTGGGGGGTCCCTGAGACTCTCCTGTGCAGCCTCTGGA | |
| | | TTCACCTTCAGTGACTACTACATGAGCTGGGTCCGCCAG | |
| | | GCTCCCGGGAAGGGGCTGGAGTGGGTCTCAGGCATTAGT | |
| | | TGGAGTAGTCGTGACAAAGGCTATGCGGACTCTGTGAAG | |
| | | GGCCGTTTCACCATCTCCAGAGACAATTCCAAGAACACG | |
| | | CTGTATCTGCAAATGAACAGCCTGAGAGCCGAGGACACT | |
| | | GCCGTGTATTACTGTACCACAGATCTCGCTAGGTACTGG | |
| | | GGCCAGGGTACACTGGTCACCGTGAGCTCAGCCTCCACC | |
| | | AAGGGCCCATCGGTCTTCCCCCTGGCACCCTCCTCCAAG | |
| | | AGCACCTCTGGGGGCACAGCGGCCCTGGGCTGCCTGGTC | |
| | | AAGGACTACTTCCCCGAACCGGTGACGGTGTCGTGGAAC | |
| | | TCAGGCGCCCTGACCAGCGGCGTGCACACCTTCCCGGCT | |
| | | GTCCTACAGTCCTCAGGACTCTACTCCCTCAGCAGCGTG | |
| | | GTGACCGTGCCCTCCAGCAGCTTGGGCACCCAGACCTAC | |
| | | ATCTGCAACGTGAATCACAAGCCCAGCAACACCAAGGTG | |
| | | GACAAGAAAGTTGAGCCCAAATCTTGTGACAAAACTCAC | |
| | | ACATGCCCACCGTGCCCAGCACCTGAACTCCTGGGGGGA | |
| | | CCGTCAGTCTTCCTCTTCCCCCCAAAACCCAAGGACACC | |
| | | CTCATGATCTCCCGGACCCCTGAGGTCACATGCGTGGTG | |
| | | GTGGACGTGAGCCACGAAGACCCTGAGGTCAAGTTCAAC | |
| | | TGGTACGTGGACGGCGTGGAGGTGCATAATGCCAAGACA | |
| | | AAGCCGCGGGAGGAGCAGTACAACAGCACGTACCGTGTG | |
| | | GTCAGCGTCCTCACCGTCCTGCACCAGGACTGGCTGAAT | |
| | | GGCAAGGAGTACAAGTGCAAGGTCTCCAACAAAGCCCTC | |
| | | CCAGCCCCCATCGAGAAAACCATCTCCAAAGCCAAAGGG | |
| | | CAGCCCCGAGAACCACAGGTGTACACCCTGCCCCCATCC | |
| | | CGGGATGAGCTGACCAAGAACCAGGTCAGCCTGACCTGC | |
| | | CTGGTCAAAGGCTTCTATCCCAGCGACATCGCCGTGGAG | |
| | | TGGGAGAGCAATGGGCAGCCGGAGAACAACTACAAGACC | |
| | | ACGCCTCCCGTGCTGGACTCCGACGGCTCCTTCTTCCTC | |
| | | TACAGCAAGCTCACCGTGGACAAGAGCAGGTGGCAGCAG | |
| | | GGGAACGTCTTCTCATGCTCCGTGATGCATGAGGCTCTG | |
| | | CACAACCACTACACGCAGAAGAGCCTCTCCCTGTCTCCG | |
| | | GGTAAATGA | |
| 4-E03 λ | 4-E03 | CAGTCTGTGCTGACTCAGCCACCCTCAGCGTCTGGGACC | 46 |
| light chain | VL | CCCGGGCAGAGGGTCACCATCTCCTGCACTGGGAGCAGC | |
| | | TCCAACATCGGGGCAGGTTATGATGTACACTGGTATCAG | |
| | | CAGCTCCCAGGAACGGCCCCCAAACTCCTCATCTATGGT | |
| | | AATGATAACCGGCCCTCAGGGGTCCCTGACCGATTCTCT | |
| | | GGCTCCAAGTCTGGCACCTCAGCCTCCCTGGCCATCAGT | |
| | | GGGCTCCGGTCCGAGGATGAGGCTGATTATTACTGTGCA | |
| | | GTATGGGATGACAGCCTGAATGGTGTGGTATTCGGCGGA | |
| | | GGAACCAAGCTGACGGTCCTAGGTCAGCCCAAGGCTGCC | |
| | | CCCTCGGTCACTCTGTTCCCGCCCTCCTCTGAGGAGCTT | |
| | | CAAGCCAACAAGGCCACACTGGTGTGTCTCATAAGTGAC | |
| | | TTCTACCCGGGAGCCGTGACAGTGGCCTGGAAGGCAGAT | |
| | | AGCAGCCCCGTCAAGGCGGGAGTGGAGACCACCACACCC | |
| | | TCCAAACAAAGCAACAACAAGTACGCGGCCAGCAGCTAT | |
| | | CTGAGCCTGACGCCTGAGCAGTGGAAGTCCCACAGAAGC | |
| | | TACAGCTGCCAGGTCACGCATGAAGGGAGCACCGTGGAG | |
| | | AAGACAGTGGCCCCTACAGAATGTTCATGA | |
| 5-B07 γ1 | 5-B07 | GAGGTGCAGCTGTTGGAGTCTGGGGGAGGCTTGGTACAG | 47 |
| heavy chain | VH | CCTGGGGGGTCCCTGAGACTCTCCTGTGCAGCCTCTGGA | |
| | | TTCACCTTCAGTAGCTATAGCATGAACTGGGTCCGCCAG | |
| | | GCTCCAGGGAAGGGGCTGGAGTGGGTCTCAGCTATTAGT | |
| | | GGTAGTGGTGGTAGCACATACTACGCAGACTCCGTGAAG | |
| | | GGCCGGTTCACCATCTCCAGAGACAATTCCAAGAACACG | |
| | | CTGTATCTGCAAATGAACAGCCTGAGAGCCGAGGACACT | |
| | | GCCGTGTATTACTGTGCGAGAGATCGGGTAGAGATGAAC | |
| | | CAGTGGCTGGCCGACTGGGGCCAGGGTACACTGGTCACC | |
| | | GTGAGCTCAGCCTCCACCAAGGGCCCATCGGTCTTCCCC | |
| | | CTGGCACCCTCCTCCAAGAGCACCTCTGGGGGCACAGCG | |
| | | GCCCTGGGCTGCCTGGTCAAGGACTACTTCCCCGAACCG | |
| | | GTGACGGTGTCGTGGAACTCAGGCGCCCTGACCAGCGGC | |
| | | GTGCACACCTTCCCGGCTGTCCTACAGTCCTCAGGACTC | |
| | | TACTCCCTCAGCAGCGTGGTGACCGTGCCCTCCAGCAGC | |
| | | TTGGGCACCCAGACCTACATCTGCAACGTGAATCACAAG | |
| | | CCCAGCAACACCAAGGTGGACAAGAAAGTTGAGCCCAAA | |
| | | TCTTGTGACAAAACTCACACATGCCCACCGTGCCCAGCA | |
| | | CCTGAACTCCTGGGGGGACCGTCAGTCTTCCTCTTCCCC | |
| | | CCAAAACCCAAGGACACCCTCATGATCTCCCGGACCCCT | |
| | | GAGGTCACATGCGTGGTGGTGGACGTGAGCCACGAAGAC | |
| | | CCTGAGGTCAAGTTCAACTGGTACGTGGACGGCGTGGAG | |
| | | GTGCATAATGCCAAGACAAAGCCGCGGGAGGAGCAGTAC | |
| | | AACAGCACGTACCGTGTGGTCAGCGTCCTCACCGTCCTG | |
| | | CACCAGGACTGGCTGAATGGCAAGGAGTACAAGTGCAAG | |
| | | GTCTCCAACAAAGCCCTCCCAGCCCCCATCGAGAAAACC | |
| | | ATCTCCAAAGCCAAAGGGCAGCCCCGAGAACCACAGGTG | |
| | | TACACCCTGCCCCCATCCCGGGATGAGCTGACCAAGAAC | |
| | | CAGGTCAGCCTGACCTGCCTGGTCAAAGGCTTCTATCCC | |
| | | AGCGACATCGCCGTGGAGTGGGAGAGCAATGGGCAGCCG | |
| | | GAGAACAACTACAAGACCACGCCTCCCGTGCTGGACTCC | |
| | | GACGGCTCCTTCTTCCTCTACAGCAAGCTCACCGTGGAC | |
| | | AAGAGCAGGTGGCAGCAGGGGAACGTCTTCTCATGCTCC | |
| | | GTGATGCATGAGGCTCTGCACAACCACTACACGCAGAAG | |
| | | AGCCTCTCCCTGTCTCCGGGTAAATGA | |
| 5-B07 λ | 5-B07 | CAGTCTGTGCTGACTCAGCCACCCTCAGCGTCTGGGACC | 48 |
| light chain | VL | CCCGGGCAGAGGGTCACCATCTCCTGCACTGGGAGCAGC | |
| | | TCCAACATCGGGGCAGGTTATGATGTACACTGGTATCAG | |
| | | CAGCTCCCAGGAACGGCCCCCAAACTCCTCATCTATAGG | |
| | | AATAATCAGCGGCCCTCAGGGGTCCCTGACCGATTCTCT | |
| | | GGCTCCAAGTCTGGCACCTCAGCCTCCCTGGCCATCAGT | |
| | | GGGCTCCGGTCCGAGGATGAGGCTGATTATTACTGTGCA | |
| | | GCATGGGATGACAGCCTGAATGGTTGGGTGTTCGGCGGA | |
| | | GGAACCAAGCTGACGGTCCTAGGTCAGCCCAAGGCTGCC | |
| | | CCCTCGGTCACTCTGTTCCCGCCCTCCTCTGAGGAGCTT | |
| | | CAAGCCAACAAGGCCACACTGGTGTGTCTCATAAGTGAC | |
| | | TTCTACCCGGGAGCCGTGACAGTGGCCTGGAAGGCAGAT | |
| | | AGCAGCCCCGTCAAGGCGGGAGTGGAGACCACCACACCC | |
| | | TCCAAACAAAGCAACAACAAGTACGCGGCCAGCAGCTAT | |
| | | CTGAGCCTGACGCCTGAGCAGTGGAAGTCCCACAGAAGC | |
| | | TACAGCTGCCAGGTCACGCATGAAGGGAGCACCGTGGAG | |
| | | AAGACAGTGGCCCCTACAGAATGTTCATGA | |
| 2-C06 γ1 | 2-C06 | GAGGTGCAGCTGTTGGAGTCTGGGGGAGGCTTGGTACAG | 49 |
| heavy chain | VH | CCTGGGGGGTCCCTGAGACTCTCCTGTGCAGCCTCTGGA | |
| | | TTCACCTTTAGCAGCTATGCCATGAGCTGGGTCCGCCAG | |
| | | GCTCCAGGGAAGGGGCTGGAGTGGGTCTCAGGTATTAGT | |
| | | GGCAGCGGTGGGTACATACACTATGCAGACTCCGTGAAG | |
| | | GGCCGGTTCACCATCTCCAGAGACAATTCCAAGAACACG | |
| | | CTGTATCTGCAAATGAACAGCCTGAGAGCCGAGGACACT | |
| | | GCCGTGTATTACTGTGCGACCTATAGCAGTGGCCTGCAT | |
| | | GATGCTTTTGATATCTGGGGCCAGGGTACACTGGTCACC | |
| | | GTGAGCTCAGCCTCCACCAAGGGCCCATCGGTCTTCCCC | |
| | | CTGGCACCCTCCTCCAAGAGCACCTCTGGGGGCACAGCG | |
| | | GCCCTGGGCTGCCTGGTCAAGGACTACTTCCCCGAACCG | |
| | | GTGACGGTGTCGTGGAACTCAGGCGCCCTGACCAGCGGC | |
| | | GTGCACACCTTCCCGGCTGTCCTACAGTCCTCAGGACTC | |
| | | TACTCCCTCAGCAGCGTGGTGACCGTGCCCTCCAGCAGC | |
| | | TTGGGCACCCAGACCTACATCTGCAACGTGAATCACAAG | |
| | | CCCAGCAACACCAAGGTGGACAAGAAAGTTGAGCCCAAA | |
| | | TCTTGTGACAAAACTCACACATGCCCACCGTGCCCAGCA | |
| | | CCTGAACTCCTGGGGGGACCGTCAGTCTTCCTCTTCCCC | |
| | | CCAAAACCCAAGGACACCCTCATGATCTCCCGGACCCCT | |
| | | GAGGTCACATGCGTGGTGGTGGACGTGAGCCACGAAGAC | |
| | | CCTGAGGTCAAGTTCAACTGGTACGTGGACGGCGTGGAG | |
| | | GTGCATAATGCCAAGACAAAGCCGCGGGAGGAGCAGTAC | |
| | | AACAGCACGTACCGTGTGGTCAGCGTCCTCACCGTCCTG | |
| | | CACCAGGACTGGCTGAATGGCAAGGAGTACAAGTGCAAG | |
| | | GTCTCCAACAAAGCCCTCCCAGCCCCCATCGAGAAAACC | |
| | | ATCTCCAAAGCCAAAGGGCAGCCCCGAGAACCACAGGTG | |
| | | TACACCCTGCCCCCATCCCGGGATGAGCTGACCAAGAAC | |
| | | CAGGTCAGCCTGACCTGCCTGGTCAAAGGCTTCTATCCC | |
| | | AGCGACATCGCCGTGGAGTGGGAGAGCAATGGGCAGCCG | |
| | | GAGAACAACTACAAGACCACGCCTCCCGTGCTGGACTCC | |
| | | GACGGCTCCTTCTTCCTCTACAGCAAGCTCACCGTGGAC | |
| | | AAGAGCAGGTGGCAGCAGGGGAACGTCTTCTCATGCTCC | |
| | | GTGATGCATGAGGCTCTGCACAACCACTACACGCAGAAG | |
| | | AGCCTCTCCCTGTCTCCGGGTAAATGA | |
| 2-C06 λ | 2-C06 | CAGTCTGTGCTGACTCAGCCACCCTCAGCGTCTGGGACC | 50 |
| light chain | VL | CCCGGGCAGAGGGTCACCATCTCCTGCACTGGGAGCAGC | |
| | | TCCAACATCGGGGCAGGTTATGATGTACACTGGTATCAG | |
| | | CAGCTCCCAGGAACGGCCCCCAAACTCCTCATCTATGAC | |
| | | AATAATAAGCGACCCTCAGGGGTCCCTGACCGATTCTCT | |
| | | GGCTCCAAGTCTGGCACCTCAGCCTCCCTGGCCATCAGT | |
| | | GGGCTCCGGTCCGAGGATGAGGCTGATTATTACTGTGCA | |
| | | GCATGGGATGACAGCCTGAATGGTTGGGTGTTCGGCGGA | |
| | | GGAACCAAGCTGACGGTCCTAGGTCAGCCCAAGGCTGCC | |
| | | CCCTCGGTCACTCTGTTCCCGCCCTCCTCTGAGGAGCTT | |
| | | CAAGCCAACAAGGCCACACTGGTGTGTCTCATAAGTGAC | |
| | | TTCTACCCGGGAGCCGTGACAGTGGCCTGGAAGGCAGAT | |
| | | AGCAGCCCCGTCAAGGCGGGAGTGGAGACCACCACACCC | |
| | | TCCAAACAAAGCAACAACAAGTACGCGGCCAGCAGCTAT | |
| | | CTGAGCCTGACGCCTGAGCAGTGGAAGTCCCACAGAAGC | |
| | | TACAGCTGCCAGGTCACGCATGAAGGGAGCACCGTGGAG | |
| | | AAGACAGTGGCCCCTACAGAATGTTCATGA | |
| 2-F09 γ1 | 2-F09 | GAGGTGCAGCTGTTGGAGTCTGGGGGAGGCTTGGTACAG | 51 |
| heavy chain | VH | CCTGGGGGGTCCCTGAGACTCTCCTGTGCAGCCTCTGGA | |
| | | TTCACCTTCAAAGCCTATAGCATGAGCTGGATCCGCCAG | |
| | | GCTCCAGGGAAGGGGCTGGAGTGGGTCTCAGGTATCAGT | |
| | | AACACGGGAGGTAGCACAGACTTCGCAGACTCCGTGAAG | |
| | | GGCCGGTTCACCATCTCCAGAGACAATTCCAAGAACACG | |
| | | CTGTATCTGCAAATGAACAGCCTGAGAGCCGAGGACACT | |
| | | GCCATGTATTACTGTGCGAGATTGGGATATAGTGGCTAC | |
| | | GACGACCGTGGTATGGACGTCTGGGGCCAAGGTACACTG | |
| | | GTCACCGTGAGCTCAGCCTCCACCAAGGGCCCATCGGTC | |
| | | TTCCCCCTGGCACCCTCCTCCAAGAGCACCTCTGGGGGC | |
| | | ACAGCGGCCCTGGGCTGCCTGGTCAAGGACTACTTCCCC | |
| | | GAACCGGTGACGGTGTCGTGGAACTCAGGCGCCCTGACC | |
| | | AGCGGCGTGCACACCTTCCCGGCTGTCCTACAGTCCTCA | |
| | | GGACTCTACTCCCTCAGCAGCGTGGTGACCGTGCCCTCC | |
| | | AGCAGCTTGGGCACCCAGACCTACATCTGCAACGTGAAT | |
| | | CACAAGCCCAGCAACACCAAGGTGGACAAGAAAGTTGAG | |
| | | CCCAAATCTTGTGACAAAACTCACACATGCCCACCGTGC | |
| | | CCAGCACCTGAACTCCTGGGGGGACCGTCAGTCTTCCTC | |
| | | TTCCCCCCAAAACCCAAGGACACCCTCATGATCTCCCGG | |
| | | ACCCCTGAGGTCACATGCGTGGTGGTGGACGTGAGCCAC | |
| | | GAAGACCCTGAGGTCAAGTTCAACTGGTACGTGGACGGC | |
| | | GTGGAGGTGCATAATGCCAAGACAAAGCCGCGGGAGGAG | |
| | | CAGTACAACAGCACGTACCGTGTGGTCAGCGTCCTCACC | |
| | | GTCCTGCACCAGGACTGGCTGAATGGCAAGGAGTACAAG | |
| | | TGCAAGGTCTCCAACAAAGCCCTCCCAGCCCCCATCGAG | |
| | | AAAACCATCTCCAAAGCCAAAGGGCAGCCCCGAGAACCA | |
| | | CAGGTGTACACCCTGCCCCCATCCCGGGATGAGCTGACC | |
| | | AAGAACCAGGTCAGCCTGACCTGCCTGGTCAAAGGCTTC | |
| | | TATCCCAGCGACATCGCCGTGGAGTGGGAGAGCAATGGG | |
| | | CAGCCGGAGAACAACTACAAGACCACGCCTCCCGTGCTG | |
| | | GACTCCGACGGCTCCTTCTTCCTCTACAGCAAGCTCACC | |
| | | GTGGACAAGAGCAGGTGGCAGCAGGGGAACGTCTTCTCA | |
| | | TGCTCCGTGATGCATGAGGCTCTGCACAACCACTACACG | |
| | | CAGAAGAGCCTCTCCCTGTCTCCGGGTAAATGA | |
| 2-F09 λ | 2-F09 | CAGTCTGTGCTGACTCAGCCACCCTCAGCGTCTGGGACC | 52 |
| light chain | VL | CCCGGGCAGAGGGTCACCATCTCTTGTTCTGGAAGCAGC | |
| | | TCCAACATCGGAAGTAATTATGTGTACTGGTATCAGCAG | |
| | | CTCCCAGGAACGGCCCCCAAACTCCTCATCTATGGTAAC | |
| | | AGCAATCGGCCCTCAGGGGTCCCTGACCGATTCTCTGGC | |
| | | TCCAAGTCTGGCACCTCAGCCTCCCTGGCCATCAGTGGG | |
| | | CTCCGGTCCGAGGATGAGGCTGATTATTACTGCCAGTCC | |
| | | TATGACAGCAGCCTGAGTGGTCCTGTGGTATTCGGCGGA | |
| | | GGAACCAAGCTGACGGTCCTAGGTCAGCCCAAGGCTGCC | |
| | | CCCTCGGTCACTCTGTTCCCGCCCTCCTCTGAGGAGCTT | |
| | | CAAGCCAACAAGGCCACACTGGTGTGTCTCATAAGTGAC | |
| | | TTCTACCCGGGAGCCGTGACAGTGGCCTGGAAGGCAGAT | |
| | | AGCAGCCCCGTCAAGGCGGGAGTGGAGACCACCACACCC | |
| | | TCCAAACAAAGCAACAACAAGTACGCGGCCAGCAGCTAT | |
| | | CTGAGCCTGACGCCTGAGCAGTGGAAGTCCCACAGAAGC | |
| | | TACAGCTGCCAGGTCACGCATGAAGGGAGCACCGTGGAG | |
| | | AAGACAGTGGCCCCTACAGAATGTTCATGA | |

In some embodiments, it is advantageous that the antibody molecule binds both to human CTLA-4 (hCTLA-4) and to cynomolgus monkey CTLA-4 (cmCTLA-4 or cyno CTLA-4). Cross-reactivity with CTLA-4 expressed on cells in cynomolgus monkey, also called crab-eating macaque or Macaca fascicularis, may be advantageous since this enables testing of the antibody molecule in monkey without having to use a surrogate antibody, which particular focus on tolerability.

In some embodiments, it is advantageous that the antibody molecule binds both to human CTLA-4 (hCTLA-4) and to murine CTLA-4 (mCTLA-4). This may be advantageous since this enables testing of the antibody molecule in mice, with particular focus on effect and pharmacodynamics, without having to use a surrogate antibody.

In some embodiments, the antibody molecule binds to all three hCTLA-4, cmCTLA-4 and mCTLA-4.

In some embodiments, it is necessary to use a surrogate antibody to test an antibody molecule's functional activity in relevant in vivo models in mice. To ensure the comparability between the antibody molecule's effect in humans and the in vivo results for the surrogate antibody in mice, it is essential to select a functionally equivalent surrogate antibody having the same in vitro characteristics as the human antibody molecule.

In some embodiments, the antibody molecule does not bind human CD28.

It would be known to the person skilled in medicine, that medicines can be modified with different additives, for example to change the rate in which the medicine is absorbed by the body; and can be modified in different forms, for example to allow for a particular administration route to the body.

Accordingly, we include that the antibody molecules, nucleotide sequences, plasmids, viruses and/or cells described herein may be combined with a pharmaceutically acceptable excipient, carrier, diluent, vehicle and/or adjuvant into a pharmaceutical composition. In this context, the term pharmaceutical composition can be used interchangeably with the terms pharmaceutical preparation, pharmaceutical formulation, therapeutic composition, therapeutic preparation, therapeutic formulation and therapeutic entity.

The pharmaceutical compositions described herein may comprise, or in some embodiments consist of, antibody molecules, nucleotide sequences, plasmids, viruses or cells.

The pharmaceutical compositions described herein may in some embodiments consist of or comprise plasmids comprising nucleotide sequences encoding the above described antibody molecules or comprising the above described nucleotide sequences.

In some embodiments, the pharmaceutical compositions may comprise nucleotide sequences encoding parts of or a complete antibody molecule described herein integrated in a cell or viral genome or in a viriome. The pharmaceutical composition may then comprise a cell or a virus as a delivery vehicle for an antibody of the invention (or a delivery vehicle for a nucleotide sequence encoding an antibody of the invention). For example, in an embodiment, the virus may be in the form of a therapeutic oncolytic virus comprising nucleotide sequences encoding at least one of the antibody molecules described herein. In some embodiments, such an oncolytic virus comprises nucleotide sequences encoding a full-length human IgG antibody. In some embodiments, such an oncolytic virus comprises nucleotide sequences encoding an scFv, Fab or F(ab′)₂ antibody molecule.

As described in the accompanying claims, in an embodiment the invention relates to a virus comprising a nucleotide sequence of the invention or a plasmid of the invention. Preferably, the virus is an oncolytic virus, such as a therapeutic oncolytic virus. As used herein, the term “oncolytic” refers to the capacity of a virus of selectively replicating in dividing cells (e.g. a proliferative cell such as a cancer cell) with the aim of slowing the growth and/or lysing said dividing cell, either in vitro or in vivo, while showing no or minimal replication in non-dividing (e.g. normal or healthy) cells. “Replication” (or any form of replication such as “replicate” and “replicating”, etc.) means duplication of a virus that can occur at the level of nucleic acid or, preferably, at the level of infectious viral particle. Such an oncolytic virus can be obtained from any member of virus identified at present time. It may be a native virus that is naturally oncolytic or may be engineered by modifying one or more viral genes so-as to increase tumour selectivity and/or preferential replication in dividing cells, such as those involved in DNA replication, nucleic acid metabolism, host tropism, surface attachment, virulence, lysis and spread (see for example Wong et al., 2010, Viruses 2: 78-106). One may also envisage placing one or more viral gene(s) under the control of event or tissue-specific regulatory elements (e.g. promoter). Exemplary oncolytic viruses include without limitation reovirus, Seneca Valley virus (SVV), vesicular stomatitis virus (VSV), Newcastle disease virus (NDV), herpes simplex virus (HSV), morbillivirus, adenovirus, poxvirus, retrovirus, measles virus, foamy virus, alpha virus, lentivirus, influenza virus, Sinbis virus, myxoma virus, rhabdovirus, picornavirus, coxsackievirus, parvovirus or the like. Such viruses are known to those skilled in the arts of medicine and virology.

In some embodiments, such an oncolytic virus is obtained from a herpes virus. The Herpesviridae are a large family of DNA viruses that all share a common structure and are composed of relatively large double-stranded, linear DNA genomes encoding 100-200 genes encapsidated within an icosahedral capsid which is enveloped in a lipid bilayer membrane. Although the oncolytic herpes virus can be derived from different types of HSV, particularly preferred are HSV1 and HSV2. The herpes virus may be genetically modified so-as to restrict viral replication in tumours or reduce its cytotoxicity in non-dividing cells. For example, any viral gene involved in nucleic acid metabolism may be inactivated, such as thymidine kinase (Martuza et al., 1991, Science 252: 854-6), ribonucleotide reductase (RR) (Mineta et al., 1994, Cancer Res. 54: 3363-66), or uracil-N-glycosylase (Pyles et al., 1994, J. Virol. 68: 4963-72). Another aspect involves viral mutants with defects in the function of genes encoding virulence factors such as the ICP34.5 gene (Chambers et al., 1995, Proc. Natl. Acad. Sci. USA 92: 1411-5). Representative examples of oncolytic herpes virus include NV1020 (e.g. Geevarghese et al., 2010, Hum. Gene Ther. 21(9): 1119-28) and T-VEC (Harrington et al., 2015, Expert Rev. Anticancer Ther. 15(12):1389-1403).

In some embodiments, such an oncolytic virus is obtained from an adenovirus. Methods are available in the art to engineer oncolytic adenoviruses. An advantageous strategy includes the replacement of viral promoters with tumour-selective promoters or modifications of the E1 adenoviral gene product(s) to inactivate its/their binding function with p53 or retinoblastoma (Rb) protein that are altered in tumour cells. In the natural context, the adenovirus E1B55 kDa gene cooperates with another adenoviral product to inactivate p53 (p53 is frequently dysregulated in cancer cells), thus preventing apoptosis. Representative examples of oncolytic adenoviruses include ONYX-015 (e.g. Khuri et al., 2000, Nat. Med 6(8): 879-85) and H101 also named Oncorine (Xia et al., 2004, Ai Zheng 23(12): 1666-70).

In some embodiments, such an oncolytic virus is a poxvirus. As used herein the term “poxvirus” refers to a virus belonging to the Poxviridae family, with a specific preference for a poxvirus belonging to the Chordopoxviridae subfamily and more preferably to the Orthopoxvirus genus. Vaccinia virus, cowpox virus, canarypox virus, ectromelia virus, myxoma virus are particularly appropriate in the context of the invention. Genomic sequences of such poxviruses are available in the art and specialized databases (e.g. Genbank under accession number NC_006998, NC_003663 or AF482758.2, NC_005309, NC_004105, NC_001132 respectively).

In specific and preferred embodiments, such an oncolytic poxvirus is an oncolytic vaccinia virus. Vaccinia viruses are members of the poxvirus family characterized by a 200 kb double-stranded DNA genome that encodes numerous viral enzymes and factors that enable the virus to replicate independently from the host cell machinery. The majority of vaccinia virus particles is intracellular (IMV for intracellular mature virion) with a single lipid envelop and remains in the cytosol of infected cells until lysis. The other infectious form is a double enveloped particle (EEV for extracellular enveloped virion) that buds out from the infected cell without lysing it. Although it can derive from any vaccinia virus strain, Elstree, Wyeth, Copenhagen, Lister and Western Reserve strains are particularly preferred. The gene nomenclature used herein is that of Copenhagen vaccinia strain unless otherwise indicated. However, correspondence between Copenhagen and other vaccinia strains are generally available in the literature.

Preferably, such an oncolytic vaccinia virus is modified by altering one or more viral gene(s). Said modification(s) preferably lead(s) to the absence of synthesis or the synthesis of a defective viral protein unable to ensure the activity of the protein produced under normal conditions by the unmodified gene. Exemplary modifications are disclosed in the literature with the goal of altering viral genes involved in DNA metabolism, host virulence, IFN pathway (e.g. Guse et al., 2011, Expert Opinion Biol. Ther.11(5):595-608) and the like. Modifications for altering a viral locus encompass deletion, mutation and/or substitution of one or more nucleotide(s) (contiguous or not) within the viral gene or its regulatory elements. Modification(s) can be made by a number of ways known to those skilled in the art using conventional recombinant techniques.

More preferably, such an oncolytic vaccinia virus is modified by altering the thymidine kinase-encoding gene (locus J2R). The thymidine kinase (TK) enzyme is involved in the synthesis of deoxyribonucleotides. TK is needed for viral replication in normal cells as these cells have generally low concentration of nucleotides whereas it is dispensable in dividing cells which contain high nucleotide concentration.

Alternatively to or in combination, such an oncolytic vaccinia virus is modified by altering at least one gene or both genes encoding ribonucleotide reductase (RR). In the natural context, this enzyme catalyses the reduction of ribonucleotides to deoxyribonucleotides that represents a crucial step in DNA biosynthesis. The viral enzyme is similar in subunit structure to the mammalian enzyme, being composed of two heterologous subunits, designed R1 and R2 encoded respectively by the I4L and F4L locus. In the context of the invention, either the I4L gene (encoding the R1 large subunit) or the F4L gene (encoding the R2 small subunit) or both may be inactivated (e.g. as described in WO2009/065546 and Foloppe et al., 2008, Gene Ther., 15:1361-71). Sequences for the J2R, I4L and F4L genes and their locations in the genome of various poxviruses are available in public databases.

In some embodiments, such an oncolytic virus comprises nucleotide sequences encoding amino acid sequence having at least 80% identity with a sequence set out in table 2 above. In some embodiments, such an oncolytic virus comprises an amino acid sequence having at least 85% identity with a sequence set out in table 2 above. In some embodiments, such an oncolytic virus comprises an amino acid sequence having at least 90% identity with a sequence set out in table 2 above. In some embodiments, such an oncolytic virus comprises an amino acid sequence having at least 95% identity with a sequence set out in table 2 above.

In some embodiments, such an oncolytic virus comprises nucleotide sequences encoding SEQ. ID. NO: 20 and ID. NO: 21. In some embodiments, such an oncolytic virus comprises nucleotide sequences encoding SEQ. ID. NO: 27 and ID. NO: 28. In some embodiments, such a oncolytic virus comprises nucleotide sequences encoding SEQ. ID. NO: 33 and ID. NO: 34. In some embodiments, such a oncolytic virus comprises nucleotide sequences encoding SEQ. ID. NO: 41 and ID. NO: 42.

In some embodiments, such an oncolytic virus comprises nucleotide sequences having at least 80% identity with a sequence set out in table 4 above. In some embodiments, such an oncolytic virus comprises nucleotide sequences having at least 85% identity with a sequence set out in table 4 above. In some embodiments, such an oncolytic virus comprises nucleotide sequences having at least 90% identity with a sequence set out in table 4 above. In some embodiments, such an oncolytic virus comprises nucleotide sequences having at least 95% identity with a sequence set out in table 4 above.

In some embodiments, such a oncolytic virus comprises SEQ. ID. NO: 45 and 46. In some embodiments, such a oncolytic virus comprises SEQ. ID. NO: 47 and 48. In some embodiments, such a oncolytic virus comprises SEQ. ID. NO: 49 and 50. In some embodiments, such a oncolytic virus comprises SEQ. ID. NO: 51 and 52.

Some oncolytic viruses have capacity to host large enough DNA insertions to accommodate integration of full-length human antibody sequences. Attenuated Vaccinia viruses and Herpes Simplex Viruses are examples of therapeutic oncolytic viruses whose genome is sufficiently large to permit integration of full-length IgG antibody sequences (Chan, W. M. et al 2014 Annu Rev Virol 1(1): 119-141; Bommareddy, P. K., et al. 2018 Nat Rev Immunol 18(8): 498-513). Full-length IgG antibodies have successfully been integrated into oncolytic Vaccinia virus, resulting in expression and extracellular release (production) of full-length IgG antibodies upon infection of virus-susceptible host cells e.g. cancer cells (Kleinpeter, P., et al. 2016, Oncoimmunology 5(10): e1220467). Adenoviruses can also be engineered to encode full-length IgG antibodies that are functionally produced and secreted upon cellular infection (Marino, N., et al. 2017 J Clin Invest 123(6): 2447-2463).

In a preferred embodiment, such an oncolytic virus is a poxvirus (e.g. a vaccinia virus) defective for TK activity (resulting from alteration of the J2R locus) or defective for both TK and RR activities (resulting from alteration of both the J2R locus and at least one of the RR-encoding I4L and/or F4L locus) and comprising (a) nucleotide sequences encoding SEQ. ID. NO: 20 and ID. NO: 21 or (b) nucleotide sequences encoding SEQ. ID. NO: 27 and ID. NO: 28 or (c) nucleotide sequences encoding SEQ. ID. NO: 33 and ID. NO: 34 or (d) nucleotide sequences encoding SEQ. ID. NO: 41 and ID. NO: 42.

When appropriate, it may be advantageous that the nucleotide sequence(s) inserted in the oncolytic virus described herein include(s) additional regulatory elements to facilitate expression, trafficking and biological activity. For example, a signal peptide may be included for facilitating secretion outside the producer cell (e.g. infected cell). The signal peptide is typically inserted at the N-terminus of the encoded polypeptide immediately after the Met initiator. The choice of signal peptides is wide and is accessible to persons skilled in the art. For example, signal peptides originating from another immunoglobin (e.g. a heavy chain IgG) can be used in the context of the invention to allow secretion of the anti-CTLA4 antibody described herein outside the producer cell. For illustrative purposes, one may refer to SEQ ID NO: 53 and SEQ ID NO:54 comprising the light chain and the heavy chain of the 4-E03 antibody described herein equipped with IgG-originating peptide signals.

A particularly preferred oncolytic virus is a vaccinia virus (e.g. Copenhagen strain) defective for both TK and RR activities (resulting from alteration of both the J2R locus and the I4L loci) and comprising nucleotide sequences encoding SEQ. ID. NO: 20 and SEQ ID. NO: 21 or SEQ. ID. NO: 53 and SEQ ID. NO: 54.

In some embodiments, such an oncolytic virus may further comprise additional nucleotide sequence(s) of therapeutic interest such as nucleotide sequence(s) encoding immunomodulatory polypeptide(s) (i.e. a polypeptide involved in stimulating an immune response either directly or indirectly). Representative examples of suitable immunomodulatory polypeptides include, without any limitation, cytokines and chemokines with a specific preference for granulocyte macrophage colony stimulating factor (GM-CSF) and particularly human, non-human primate or murine GM-CSF. The additional nucleotide sequence may be easily obtained by standard molecular biology techniques (e.g. PCR amplification, cDNA cloning, chemical synthesis) using sequence data accessible in the art and the information provided herein. A particularly preferred oncolytic virus is a vaccinia virus (e.g. Copenhagen strain) defective for both TK and RR activities (resulting from alteration of both the J2R locus and the I4L loci) and comprising nucleotide sequences encoding SEQ. ID. NO: 20 and ID. NO: 21 or SEQ. ID. NO: 53 and SEQ ID. NO: 54 and a nucleotide sequence encoding a GM-CSF, with a specific preference for a human GM-CSF (e.g. having SEQ ID NO: 55 or SEQ ID NO: 56) or a murine GM-CSF (e.g. having SEQ ID NO: 57 or SEQ ID NO: 58).

In addition, the nucleotide sequences to be inserted in such an oncolytic virus can be optimized for providing high level expression in a particular host cell or subject by modifying one or more codon(s). Further to optimization of the codon usage, various modifications may also be envisaged so as to prevent clustering of rare, non-optimal codons being present in concentrated areas and/or to suppress or modify “negative” sequence elements which are expected to negatively influence expression levels. Such negative sequence elements include without limitation the regions having very high (>80%) or very low (<30%) GC content; AT-rich or GC-rich sequence stretches; unstable direct or inverted repeat sequences; R A secondary structures; and/or internal cryptic regulatory elements such as internal TATA-boxes, chi-sites, ribosome entry sites, and/or splicing donor/acceptor sites.

In some embodiments, the nucleotide sequence(s) are placed under the control of suitable regulatory elements for their proper expression in a host cell or subject. As used herein, the term “regulatory elements” refers to any element that allows, contributes or modulates the expression of the encoding nucleotide sequence(s) in a given host cell or subject, including their replication, duplication, transcription, splicing, translation, stability and/or transport in or outside the expressing cell. It will be appreciated by those skilled in the art that the choice of the regulatory elements can depend on such factors as the nucleotide sequence itself, the virus into which it is inserted, the host cell or subject, the level of expression desired, etc. The promoter is of special importance. In the context of the invention, it can be constitutive directing expression of the nucleotide sequence that it controls in many types of host cells or specific to certain host cells or regulated in response to specific events or exogenous factors (e.g. by temperature, nutrient additive, hormone, etc.) or according to the phase of a viral cycle (e.g. late or early). Promoters adapted to virus-mediated expression are known in the art. Representative examples for expression by an oncolytic poxvirus include without limitation the vaccinia p7.5K, pH5.R, p11K7.5, TK, p28, p11, pB2R, pA35R, K1L and pSE/L promoters (Erbs et al., 2008, Cancer Gene Ther. 15 (1): 18-28; Orubu et al. 2012, PloS One 7: e40167), early/late chimeric promoters and synthetic promoters (Chakrabarti et al., 1997, Biotechniques 23: 1094-7; Hammond et al, 1997, J. Virol Methods 66: 135-8; and Kumar and Boyle, 1990, Virology 179:151-8). In preferred embodiments, the nucleotide sequences of the light and heavy chains of the antibody described herein are respectively placed under the control of promoters having the same transcriptional strength, and preferably under the control of the same promoter (e.g. p7.5K such as the one described in SEQ ID NO: 59 or pH5.R such as the one described in SEQ ID NO: 60) to obtain a similar level of expression for both chains and therefore an optimal assembly of the antibody as a hetero-tetrameric protein (i.e. to avoid excess of non-associated chain). The additional nucleotide sequence (e.g. encoding GM-CSF) can be placed under a different promoter (e.g. pSE/L such as the one described in SEQ ID NO: 61).

Insertion of the nucleotide sequence(s) (possibly equipped with appropriate regulatory elements) in genome of such an oncolytic virus is made by conventional means, either using appropriate restriction enzymes or, preferably by homologous recombination. The nucleotide sequence(s) can independently be inserted at any location of the viral genome. Various sites of insertion may be considered, e.g. in a non-essential viral gene, in an intergenic region, or in a non-coding portion of the genome of such an oncolytic virus. J2R locus and/or I4L locus is particularly appropriate for an oncolytic virus being a poxvirus (e.g. a oncolytic vaccinia virus). Upon insertion of the nucleotide sequence(s) into the viral genome, the viral locus at the insertion site may be deleted at least partially. In one embodiment, this deletion or partial deletion may result in suppressed expression of the viral gene product encoded by the entirely or partially deleted locus resulting in a defective virus for said virus function. A particularly preferred oncolytic virus is a TK and/or RR defective vaccinia virus comprising the cassette encoding the heavy chain inserted at the J2R locus and the cassette encoding the light chain inserted at the I4L locus. The cassette encoding the additional GM-CSF-encoding nucleotide sequence can be inserted in another location of the virus genome or in J2R or I4L locus, with a preference for insertion at the I4L locus.

The present invention also provides a method for generating such an oncolytic virus described herein, and particularly an oncolytic poxvirus, into a suitable host cell (producer cell). In some embodiments, such a method comprises one or more step(s) of homologous recombination between a virus genome and a transfer plasmid comprising the nucleotide sequence(s) to be inserted (possibly with regulatory elements) flanked in 5′ and 3′ with viral sequences respectively present upstream and downstream the insertion site. Said transfer plasmid can be generated and introduced into the host cell by routine techniques (e.g. by transfection). The virus genome can be introduced into the host cell by infection. The size of each flanking viral sequence may vary from at least 100 bp and at most 1500 bp on each side of the nucleotide sequence (preferably from 200 to 550 bp and more preferably from 250 to 500 bp). Homologous recombination permitting to generate such an oncolytic virus is preferably carried out in cultured cell lines (e.g. HeLa, Vero) or in chicken embryonic fibroblasts (CEF) cells obtained from embryonated eggs.

In some embodiments, the identification of the oncolytic virus having incorporated the anti-CTLA4 encoding nucleotide sequences and possibly the additional nucleotide sequence (e.g. GM-CSF) may be facilitated by the use of a selection and/or a detectable gene. In preferred embodiments, the transfer plasmid further comprises a selection marker with a specific preference for the GPT gene (encoding a guanine phosphoribosyl transferase) permitting growth in a selective medium (e.g. in the presence of mycophenolic acid, xanthine and hypoxanthine) or a detectable gene encoding a detectable gene product such as GFP, e-GFP or mCherry. In addition, the use of an endonuclease capable of providing a double-stranded break in said selection or detectable gene may also be considered. Said endonuclease may be in the form of a protein or expressed by an expression vector.

Once generated, such an oncolytic virus can be amplified into a suitable host cell using conventional techniques including culturing the transfected or infected host cell under suitable conditions so as to allow the production and recovery of infectious particles.

The present invention also relates to a method for producing the oncolytic virus described herein. Preferably said method comprises the steps of a) preparing a producer cell line, b) transfecting or infecting the prepared producer cell line with the oncolytic virus, c) culturing the transfected or infected producer cell line under suitable conditions so as to allow the production of the virus, d) recovering the produced virus from the culture of said producer cell line and optionally e) purifying said recovered virus.

In some embodiments, the producer cell is selected from the group consisting of mammalian (e.g. human or non-human) cells such as Hela cells (e.g. ATCC-CRM-CCL-2™ or ATCC-CCL-2.2™), HER96, PER-C6 (Fallaux et al., 1998, Human Gene Ther. 9:1909-17), hamster cell lines such as BHK-21 (ATCC CCL-10) etc. and avian cells such as those described in WO2005/042728, WO2006/108846, WO2008/129058, WO2010/130756, WO2012/001075 as well as a primary chicken embryo fibroblast (CEF) prepared from chicken embryos obtained from fertilized eggs. Producer cells are preferably cultured in an appropriate medium which can, if needed, be supplemented with serum and/or suitable growth factor(s) or not (e.g. a chemically defined medium preferably free from animal- or human-derived products). An appropriate medium may be easily selected by those skilled in the art depending on the producer cells. Such media are commercially available. Producer cells are preferably cultured at a temperature comprised between +30° C. and +38° C. (more preferably at approximately +37° C.) for between 1 and 8 days before infection. If needed, several passages of 1 to 8 days may be made in order to increase the total number of cells.

In step b), producer cells are infected by the oncolytic virus under appropriate conditions using an appropriate multiplicity of infection (MOI) to permit productive infection of producer cells. For illustrative purposes, an appropriate MOI ranges from 10-3 to 20, with a specific preference for a MOI comprises from 0.01 to 5 and more preferably 0.03 to 1. Infection step is carried out in a medium which may be the same as or different from the medium used for the culture of producer cells.

In step c), infected producer cells are then cultured under appropriate conditions well known to those skilled in the art until progeny virus particles is produced. Culture of infected producer cells is also preferably performed in a medium which may be the same as or different from the medium/media used for culture of producer cells and/or for infection step, at a temperature between +32° C. and +37° C., for 1 to 5 days.

In step d), the virus particles produced in step c) are collected from the culture supernatant and/or the producer cells. Recovery from producer cells may require a step allowing the disruption of the producer cell membrane to allow the liberation of the virus. The disruption of the producer cell membrane can be induced by various techniques well known to those skilled in the art, including but not limited to freeze/thaw, hypotonic lysis, sonication, microfluidization, high shear (also called high speed) homogenization or high-pressure homogenization.

The recovered oncolytic virus may be at least partially purified before being distributed in doses and used as described herein. A vast number of purification steps and methods is available in the art, including e.g. clarification, enzymatic treatment (e.g. endonuclease, protease, etc.), chromatographic and filtration steps. Appropriate methods are described in the art (see e.g. WO2007/147528; WO2008/138533, WO2009/100521, WO2010/130753, WO2013/022764).

In one embodiment, the present invention also provides a cell infected with the oncolytic virus described herein.

The invention also encompasses pharmaceutical compositions comprising a virus, such as an oncolytic virus, as discussed above, and a pharmaceutically acceptable diluent, vehicle and/or an adjuvant.

The pharmaceutical composition may in some embodiments be in the form of a CAR-T cell, carrying parts or the complete antibody sequences described herein as part of the sequence coding for its chimeric antigen T cell receptor.

The invention also encompasses pharmaceutical compositions comprising a CAR-T cell as discussed above and a pharmaceutically acceptable diluent, vehicle and/or an adjuvant.

The invention also comprises other therapeutic modalities, or “shapes” of drugs, such as antibody drug conjugates, fusion proteins etc, and pharmaceutical composition comprising such therapeutic modalities.

The antibody molecules, nucleotide sequences, plasmids, viruses, cells and/or pharmaceutical compositions described herein may be suitable for parenteral administration including aqueous and/or non-aqueous sterile injection solutions which may contain anti-oxidants, and/or buffers, and/or bacteriostats, and/or solutes which render the formulation isotonic with the blood of the intended recipient; and/or aqueous and/or non-aqueous sterile suspensions which may include suspending agents and/or thickening agents. The antibody molecules, nucleotide sequences, plasmids, cells and/or pharmaceutical compositions described herein may be presented in unit-dose or multi-dose containers, for example sealed ampoules and vials, and may be stored in a freeze-dried (i.e. lyophilised) condition requiring only the addition of the sterile liquid carrier, for example water for injections, immediately prior to use.

Extemporaneous injection solutions and suspensions may be prepared from sterile powders, and/or granules, and/or tablets of the kind previously described.

For parenteral administration to human patients, the daily dosage level of the anti-CTLA-4 antibody molecule will usually be from 1 mg/kg bodyweight of the patient to 20 mg/kg, or in some cases even up to 100 mg/kg administered in single or divided doses. Lower doses may be used in special circumstances, for example in combination with prolonged administration. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

Typically, a pharmaceutical composition (or medicament) described herein comprising an antibody molecule will contain the anti-CTLA-4 antibody molecule at a concentration of between approximately 2 mg/ml and 150 mg/ml or between approximately 2 mg/ml and 200 mg/ml. In some embodiments, the pharmaceutical compositions will contain the anti-CTLA-4 antibody molecule at a concentration of 10 mg/ml.

Typically, a pharmaceutical composition (or medicament) will contain the oncolytic virus described herein at a concentration of between approximately 10³ to 10¹² vp (viral particles), iu (infectious unit) or pfu (plaque-forming units) depending on the virus and quantitative technique. The quantity of pfu present in a sample can be determined by counting the number of plaques following infection of permissive cells (e.g. CEF or Vero cells) to obtain a plaque forming units (pfu) titer, the quantity of vp by measuring the A260 absorbance, and the quantity of iu by quantitative immunofluorescence, e.g. using anti-virus antibodies. As a general guidance, individual doses which are suitable for a pharmaceutical composition comprising an oncolytic poxvirus range from approximately 10³ to approximately 10¹⁰ pfu, advantageously from approximately 10³ pfu to approximately 10⁹ pfu, preferably from approximately 10⁴ pfu to approximately 10⁸ pfu; and more preferably from approximately 10⁴ pfu to approximately 10⁷ pfu.

Generally, in humans, oral or parenteral administration of the antibody molecules, nucleotide sequences, plasmids, viruses, cells and/or pharmaceutical compositions described herein is the preferred route, being the most convenient. For veterinary use, the antibody molecules, nucleotide sequences, plasmids, viruses, cells and/or pharmaceutical compositions described herein are administered as a suitably acceptable formulation in accordance with normal veterinary practice and the veterinary surgeon will determine the dosing regimen and route of administration which will be most appropriate for a particular animal. Thus, the present invention provides a pharmaceutical formulation comprising an amount of an antibody molecule, nucleotide sequences plasmid, virus and/or cell of the invention effective to treat various conditions (as described above and further below). Preferably, the antibody molecules, nucleotide sequences, plasmids, viruses, cells and/or pharmaceutical compositions described herein is adapted for delivery by a route selected from the group comprising: intravenous; intratumoral; intramuscular; subcutaneous. Administration can be in the form of a single injection or several repeated injections (e.g with the same or different doses, with the same or different routes, at the same or different sites of administration). For illustrative purposes, individual doses comprising approximately 10⁴, 5×10⁴, 10⁵, 5×10⁵, 10⁶, 5×10⁶, 10⁷, 5×10⁷, 10⁸, 5×10⁸, 10⁹, 5×10⁹ or 10¹⁰ pfu of an oncolytic poxvirus (e.g. the TK and RR-defective vaccinia virus described herein) are particularly suited for intratumoral administration.

The present invention also includes antibody molecules, nucleotide sequences, plasmids, viruses, cells and/or pharmaceutical compositions described herein comprising pharmaceutically acceptable acid or base addition salts of the polypeptide binding moieties of the present invention. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others. Pharmaceutically acceptable base addition salts may also be used to produce pharmaceutically acceptable salt forms of the agents according to the present invention. The chemical bases that may be used as reagents to prepare pharmaceutically acceptable base salts of the present agents that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others. The antibody molecules, nucleotide sequences, plasmids, viruses and/or cells described herein may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilised (freeze dried) polypeptide binding moiety loses no more than about 20%, or no more than about 25%, or no more than about 30%, or no more than about 35%, or no more than about 40%, or no more than about 45%, or no more than about 50% of its activity (prior to lyophilisation) when re-hydrated.

In some embodiments, the viral composition is suitably buffered at a physiological or slightly basic pH (e.g. from approximately pH 7 to approximately pH 9 with a specific preference for a pH comprised between 7 and 8.5 and more particularly close to 8). It might be beneficial to also include in the viral composition a monovalent salt so as to ensure an appropriate osmotic pressure. Said monovalent salt may notably be selected from NaCl and KCl, preferably said monovalent salt is NaCl, preferably in a concentration of 10 to 500 mM (e.g 50 mM). A suitable viral composition comprises saccharose 50 g/L, NaCl 50 mM, Tris-HCl 10 mM and Sodium glutamate 10 mM, pH8. The composition may also be formulated so as to include a cryoprotectant for protecting the oncolytic virus at low storage temperature. Suitable cryoprotectants include without limitation sucrose (or saccharose), trehalose, maltose, lactose, mannitol, sorbitol and glycerol, preferably in a concentration of 0.5 to 20% (weight in g/volume in L, referred to as w/v) as well as high molecular weight polymers such as dextran or polyvinylpyrrolidone (PVP).

The anti-CTLA-4 antibody molecules, nucleotide sequences and pharmaceutical compositions described herein can be used use in the treatment of cancer in a subject.

We include that the subject could be mammalian or non-mammalian. Preferably, the mammalian subject is a human or is a non-mammalian, such as a horse, or a cow, or a sheep, or a pig, or a camel, or a dog, or a cat. Most preferably, the mammalian subject is a human.

By “exhibit”, we include that the subject displays a cancer symptom and/or a cancer diagnostic marker, and/or the cancer symptom and/or a cancer diagnostic marker can be measured, and/or assessed, and/or quantified.

It would be readily apparent to the person skilled in medicine what the cancer symptoms and cancer diagnostic markers would be and how to measure and/or assess and/or quantify whether there is a reduction or increase in the severity of the cancer symptoms, or a reduction or increase in the cancer diagnostic markers; as well as how those cancer symptoms and/or cancer diagnostic markers could be used to form a prognosis for the cancer.

Cancer treatments are often administered as a course of treatment, which is to say that the therapeutic agent is administered over a period of time. The length of time of the course of treatment will depend on a number of factors, which could include the type of therapeutic agent being administered, the type of cancer being treated, the severity of the cancer being treated, and the age and health of the subject, amongst others reasons.

By “during the treatment”, we include that the subject is currently receiving a course of treatment, and/or receiving a therapeutic agent, and/or receiving a course of a therapeutic agent.

In some embodiments the cancer to be treated in accordance with the present invention is a solid tumour.

In some embodiments, the cancer is selected from the group consisting of advanced solid tumour, melanoma and other malignant neoplasms of skin, synovial sarcoma, non-small cell lung cancer (NSCLC), small cell lung cancer (SCLC), bladder cancer, prostate cancer, mesothelioma, ovarian cancer, breast cancer, renal cell cancer, hepatocellular carcinoma, head and neck cancer, and colorectal cancer.

Each one of the above described cancers is well-known, and the symptoms and cancer diagnostic markers are well described, as are the therapeutic agents used to treat those cancers. Accordingly, the symptoms, cancer diagnostic markers, and therapeutic agents used to treat the above mentioned cancer types would be known to those skilled in medicine.

Clinical definitions of the diagnosis, prognosis and progression of a large number of cancers rely on certain classifications known as staging. Those staging systems act to collate a number of different cancer diagnostic markers and cancer symptoms to provide a summary of the diagnosis, and/or prognosis, and/or progression of the cancer. It would be known to the person skilled in oncology how to assess the diagnosis, and/or prognosis, and/or progression of the cancer using a staging system, and which cancer diagnostic markers and cancer symptoms should be used to do so.

By “cancer staging”, we include the Rai staging, which includes stage 0, stage I, stage II, stage III and stage IV, and/or the Binet staging, which includes stage A, stage B and stage C, and/or the Ann Arbour staging, which includes stage I, stage II, stage III and stage IV.

It is known that cancer can cause abnormalities in the morphology of cells. These abnormalities often reproducibly occur in certain cancers, which means that examining these changes in morphology (otherwise known as histological examination) can be used in the diagnosis or prognosis of cancer. Techniques for visualizing samples to examine the morphology of cells, and preparing samples for visualization, are well known in the art; for example, light microscopy or confocal microscopy.

By “histological examination”, we include the presence of small, mature lymphocyte, and/or the presence of small, mature lymphocytes with a narrow border of cytoplasm, the presence of small, mature lymphocytes with a dense nucleus lacking discernible nucleoli, and/or the presence of small, mature lymphocytes with a narrow border of cytoplasm, and with a dense nucleus lacking discernible nucleoli, and/or the presence of atypical cells, and/or cleaved cells, and/or prolymphocytes.

It is well known that cancer is a result of mutations in the DNA of the cell, which can lead to the cell avoiding cell death or uncontrollably proliferating. Therefore, examining these mutations (also known as cytogenetic examination) can be a useful tool for assessing the diagnosis and/or prognosis of a cancer. An example of this is the deletion of the chromosomal location 13q14.1 which is characteristic of chronic lymphocytic leukaemia. Techniques for examining mutations in cells are well known in the art; for example, fluorescence in situ hybridization (FISH).

By “cytogenetic examination”, we include the examination of the DNA in a cell, and, in particular the chromosomes. Cytogenetic examination can be used to identify changes in DNA which may be associated with the presence of a refractory cancer and/or relapsed cancer. Such may include: deletions in the long arm of chromosome 13, and/or the deletion of chromosomal location 13q14.1, and/or trisomy of chromosome 12, and/or deletions in the long arm of chromosome 12, and/or deletions in the long arm of chromosome 11, and/or the deletion of 11q, and/or deletions in the long arm of chromosome 6, and/or the deletion of 6q, and/or deletions in the short arm of chromosome 17, and/or the deletion of 17p, and/or the t(11:14) translocation, and/or the (q13:q32) translocation, and/or antigen gene receptor rearrangements, and/or BCL2 rearrangements, and/or BCL6 rearrangements, and/or t(14:18) translocations, and/or t(11:14) translocations, and/or (q13:q32) translocations, and/or (3:v) translocations, and/or (8:14) translocations, and/or (8:v) translocations, and/or t(11:14) and (q13:q32) translocations.

It is known that subjects with cancer exhibit certain physical symptoms, which are often as a result of the burden of the cancer on the body. Those symptoms often reoccur in the same cancer, and so can be characteristic of the diagnosis, and/or prognosis, and/or progression of the disease. A person skilled in medicine would understand which physical symptoms are associated with which cancers, and how assessing those physical systems can correlate to the diagnosis, and/or prognosis, and/or progression of the disease. By “physical symptoms”, we include hepatomegaly, and/or splenomegaly.

### BRIEF DESCRIPTION OF THE DRAWINGS

In the examples below, reference is made to the following figures:

FIG. 1: Antibodies of the invention specifically bind CTLA-4

The antibodies were shown by ELISA to bind to human and cynomolgous CTLA-4 but not human CD28 protein. Binding for 2-C06 (FIG. 1A), 4-E-03 (FIG. 1B), 5-B07 (FIG. 1C) was compared to Yervoy (FIG. 1D).

FIG. 2: Dose-dependent binding of anti-CTLA-4 mAb to hCTLA-4-transfected cells

Anti-CTLA-4 mAb (FIG. 2A-2D) show strong binding to CTLA-4-expressing 293T cells similar to Yervoy (FIG. 2E).

FIG. 3: Anti-CTLA-4 mAb bind to in vitro-activated human CD4+ T cells

CD4+ T cells obtained from peripheral human blood were stimulated in vitro. Binding of anti-CTLA-4 mAb (solid lines, top row) was analysed by FACS and compared to Yervoy (dotted line, top row) and a commercial FACS antibody (bottom row).

FIG. 4: Binding block on in vitro-activated CD4+ T cells

Human in vitro-activated CD4+ T cells were stained with Alexa 647-labelled anti-CTLA-4 mAb (black line). Antibody binding was blocked by rhCTLA-4-Fc protein (grey line).

FIG. 5: Binding to in vitro-activated cynomologous CD4+ T cells

CD4+ T cells obtained from peripheral cynomologous blood were stimulated in vitro with CD3/CD28 dynabeads. Binding of anti-CTLA-4 mAb (solid lines, top row) was analysed by FACS and compared to Yervoy (dotted line, top row) and a commercial FACS antibody (bottom row).

FIG. 6: Block of cell binding by human and cynomologous CTLA-4 protein

293T-CTLA-4 cells were stained with Alexa 647-labelled anti-CTLA-4 mAb (black line). Antibody binding was blocked by rhCTLA-4-Fc protein (light grey line) and rcmCTLA-4-Fc protein (dark grey line).

FIG. 7: Binding to 293T cells expressing cynomologous CTLA-4

293T cells were transiently transfected with cynomologous CTLA-4 and binding of anti-CTLA-4 mAb at different concentrations was analysed by FACS.

FIG. 8: Expected lack of binding to resting human/cynomologous PBMCs

4-E03—as well as 2-C06, 5-B07 and 2-F09 (data not shown)—does not show any unspecific binding to different cell subsets in human (top row) and cynomologous (bottom row) PBMCs as analysed by FACS.

FIG. 9: Expected lack of direct agonistic activity

CFSE-labelled CD4+ T cells from healthy donors were stimulated with coated anti-CD3 plus soluble anti-CTLA-4 mAb or anti-CD28. % dividing cells (CFSElow CD25+ cells) were determined after 3 days by FACS. (A) FACS plots of one representative experiment (B) summarizing graph of 6 donors.

FIG. 10: CD80/CD86 blocking activity

Anti-CTLA-4 mAb block the binding of CD80 (FIG. 10A) and CD86 (FIG. 10B) to its ligand CTLA-4 as shown by ELISA.

FIG. 11: Functional ligand block in vitro

PBMCs were stimulated with SEB plus titrating doses of anti-CTLA-4 antibodies. Amount of secreted IL-2 in the supernatant was determined by MSD. In this figure, 1 representative donor out of 6 is shown.

FIG. 12: ADCC assay on in vitro-activated CD4+ T cells

In vitro-activated CD4+ T cells from heathy donors pre-opsonized with anti-CTLA-4 mAbs at 10 μg/ml were co-cultured with NK cells (NK-92 cell line) at 2:1 ratio. ADCC activity was assessed by FACS as described below. The figure shows the mean+SD of 4-8 donors.

FIG. 13: CTLA-4 is highest expressed on tumour resident Treg cells.

Samples of freshly excised ovarian tumours and blood were obtained from patients at surgery. Ascites was collected from patients with different cancer indications. CTLA-expression on this patient material was compared to healthy PBMCs. Tumour samples were minced and digested. Peripheral blood mononuclear cells were separated by centrifugation. CTLA-4 expression was assessed on CD4+CD25+CD127− Treg cells, CD4+ non-Treg cells and CD8+ effector T cells by flow cytometry. Data represent individual patients/donors with n=12 for healthy PBMCs, n=20 for ascites, n=9 for tumour and n=5 for patient blood.

CTLA-4 expression was also analysed on human T cells which were activated in NOG mice in vivo and then isolated from the spleen of these mice (see FIG. 14).

FIG. 14: Anti-CTLA-4 mAb mediate Treg depletion in vivo.

Human PBMCs were injected i.v. into NOG mice. After approximately 2 weeks, spleens were taken and expression of CTLA-4 on human Treg cells and CD8+ T cells was analysed by FACS. Splenic cells isolated from NOG mice were transferred i.p. into SCID mice. 1h later, mice were treated i.p. with CTLA-4 hIgG1 or control mAb. Intraperitoneal fluid was collected after 24 h and frequency of human T cell subsets (14A: Tregs and 14B: CD8+ T cells) was determined by flow cytometry.

FIG. 15: Table summarizing characteristics for anti-CTLA-4 antibodies

FIG. 16: FIG. 16: Characterization of mouse surrogate anti-CTLA-4 mAb

FIG. 16A-B: Blocking ELISAs were performed with m5-B07 to evaluate ligand blocking characteristics. The antibody blocks the binding of FIG. 16A) CD80 and FIG. 16B) CD86 to its ligand CTLA-4 in a dose-dependent manner.

FIG. 16 C-D: 5-B07 in mouse IgG2a format mediated Treg deletion in CT26 tumor model. Balb/c mice were injected subcutaneously with 1×10⁶ CT26 cells and treatment started at tumor size of ca 7×7 mm. After 3 injections of 10 mg/kg antibody, tumor single cell suspensions were analyzed for immune cell content by FACS. FIG. 16C: Ligand blocking surrogate antibody 5-B07 causes T reg depletion. This causes a shift in CD8⁺/Treg T cell ratio as depicted in FIG. 16D.

FIG. 17: Generation of COPTG19384 and COPTG19385

Schematic representation of COPTG19384 and COPTG19385 used in this study. COPTG19385 contains a deletion of J2R gene in TK locus replaced by the heavy chain of anti-CTLA-4 driven by p7.5K, and a deletion of I4L gene in RR locus replaced by the light chain of anti-CTLA-4 driven by p7.5K. COPTG19384 contains a deletion of J2R gene in TK locus replaced by the heavy chain of anti-CTLA-4 driven by p7.5K, and a deletion of I4L gene in RR locus replaced by the light chain of anti-CTLA-4 driven by p7.5K, and by GM-CSF driven by pSE/L.

FIG. 18: Expression analysis of 4-E03 monoclonal antibody in supernatant of CEF cells infected with COPTG19384

A) by Western Blot: CEF cells were infected at MOI 0.05 with COPTG19384 in triplicate. Cell supernatants were harvested after 48 h and were analysed by WB after an electrophoresis in non-reducing condition and using either an anti-Ig (left blot) or an anti-light chain (right blot) HRP conjugated antibody.

B) by ELISA: CEF cells were infected at MOI 0.05 with COPTG19384 in triplicate or VVTG17137. Cell supernatants were harvested after 48 h and were analysed by ELISA for detection of either 4-E03 monoclonal antibody.

FIG. 19: Expression analysis of GM-CSF in supernatant of CEF cells infected with COPTG19384 by ELISA

CEF cells were infected at MOI 0.05 with COPTG19384 primary research stock in triplicate or VVTG17137. Cell supernatants were harvested after 48 h and were analysed by ELISA for detection of GM-CSF.

FIG. 20: Replication studies of COP WT, COPTG19384 (two batches) and VVTG17137 (two batches) on normal and tumoral hepatocytes

A) Replication rate on normal human hepatocytes.

B) Replication rate on malignant HepG2.

C) Therapeutic indexes calculated from the replication rates measured on HepG2 and hepatocytes.

FIG. 21: Replication of COPTG19384 and VVTG17137 in reconstructed human skin

Replication of COPTG19384 and VVTG17137 were evaluated after 7 days and with inoculums varying from 10 to 10⁵ pfu. Results are the means and SEM of three measurements.

FIG. 22: Oncolytic activities of COPTG19384 and VVTG17137 in three human tumoral cell lines: MIA PaCa-2 (A), LoVo (B) and HepG2 (C)

FIG. 23: Expression level of both 4-E03 monoclonal antibody and GM-CSF in (A) supernatants of infected HepG2 and LoVo and (B) supernatants of 5 different infected human tumoral cell lines

A) 4-E03 and GM-CSF expression levels were evaluated after 5 days of incubation, at MOI from 10⁻⁵ to 10⁻² for COPTG19384 and at MOI of 10⁻² for VVTG17137 used as negative control.

B) 4-E03 and GM-CSF expression levels were evaluated 48 hours after infection by COPTG19384 at MOI 0.05.

FIG. 24: Binding of different batches of 4-E03 to CTLA-4 protein

Binding of recombinantly produced 4-E03 by CHO (4-E03 research batch) or HEK (4-E03 tox batch) cells to (A) human and (B) cynomolgus recombinant protein was compared to the binding of 4-E03 purified from the supenatant of infected MIA PaCa-2 tumor cells (4-E03 TG) by ELISA.

FIG. 25: Binding of different batches of 4-E03 to CTLA-4 expressing cells

Binding of recombinantly produced 4-E03 by CHO (4-E03 research batch) or HEK (4-E03 tox batch) cells to (A) human and (B) cynomolgus CTLA-4 expressing cells was compared to the binding of 4-E03 purified from the supenatant of infected MIA PaCa-2 tumor cells (4-E03 TG) by flow cytometry.

FIG. 26: Kinetic of virus accumulation in LoVo xenografted tumor

Kinetic of virus accumulation was evaluated in LoVo xenografted tumor after a single i.t. injection of either COPTG19384 or VVTG17137 at two different doses (10⁴ or 10⁵ pfu). The solid or dashed lines represent the median of the three values determined at each time points.

FIG. 27: Kinetic of 4-E03 mAb and GM-CSF accumulation in LoVo xenografted tumor

A) Kinetic of 4-E03 mAb accumulation in LoVo xenografted tumor was evaluated after a single i.t. injection of either COPTG19384 or VVTG17137 at two different doses (10⁴ or 10⁵ pfu) or after a single i.p. injection of 3 mg/kg of 4-E03 monoclonal antibody. The solid or dashed lines represent the median of the three values.

B) Kinetic of GM-CSF accumulation in LoVo xenografted tumor was evaluated after a single i.t. injection of either COPTG19384 at two different doses (10⁴ or 10⁵ pfu) or VVTG17137 (10⁵ pfu). The solid lines represent the median of the three values determined at each time points.

FIG. 28: Kinetic of 4-E03 mAb and GM-CSF concentrations in sera of LoVo xenografted mice

A) Kinetic of 4-E03 mAb concentrations in sera was evaluated after a single i.t. injection in LoVo xenografted tumor of either COPTG19384 or VVTG17137 at two different doses (10⁴ or 10⁵ pfu) or after a single i.p. injection of 3 mg/kg of 4-E03 monoclonal antibody. The solid lines represent the median of the three values.

B) Kinetic of GM-CSF concentrations in sera after a single i.t. injection in LoVo xenografted tumor of either COPTG19384 at two different doses (104 or 105 pfu) or VVTG17137 (105 pfu). The dashed lines represent the median of the three values determined at each time points.

FIG. 29: Kinetic of virus accumulation in CT26 tumors

Kinetic of virus accumulation in CT26 tumors after three i.t. injections (at D0, D2 and D4) of either VVTG18058, COPTG19421 or COPTG19407 at 10⁷ pfu/injection.

FIG. 30: Kinetic of m5-B07 mAb and mGM-CSF accumulation in CT26 tumor

A) Kinetic of m5-B07 mAb concentrations in CT26 tumor during and after three i.t. injections of either VVTG18058, COPTG19421 or COPTG19407 (10⁷ pfu/injection) or after a single i.p. injection of 3 mg/kg of m5-B07 monoclonal antibody. The solid lines represent the median of the three values.

B) Kinetic of mGM-CSF concentrations in CT26 tumor during and after three i.t. injections of either VVTG18058, COPTG19421 or COPTG19407 (10⁷ pfu/injection) or after a single i.p. injection of 3 mg/kg of m5-B07 monoclonal antibody. The solid lines represent the median of the three values determined at each time points.

FIG. 31: Kinetic of m5-B07 mAb concentrations in sera of CT26 model

Kinetic of m5-B07 mAb concentrations in sera after three i.t. injections in CT26 tumor of either VVTG18058, COPTG19421 or COPTG19407 (10⁷ pfu/injection) or after a single i.p. injection of 3 mg/kg of m5-B07 monoclonal antibody. The solid lines represent the median of the three values determined at each time points.

FIG. 32: Antitumoral activity in CT26 model: effect of COPTG19347+/−anti-PD1 on CT26 tumor growth (A) and mice survival (B)

CT26 cells were injected SC in BalB/c mice at D-7. COPTG19347 (107 pfu),

VVTG18058 (10⁷ pfu), VVTG18058 or buffer were injected IT, at D0, D2 and D4, possibly followed by the injection i.p. of 250 μg/mouse of anti-PD1 RMPI-14 at D7, D10, D14, D17 and D21.

FIG. 33: Dose-effect evaluation in CT26 model (compilation of survival data observed after treatment of COPTG19407, COPTG19421 and VVTG18058)

FIG. 34: Anti-tumoral activity of COPTG19407 compared to VVTG18058 plus m5-B07 in CT26 tumor model

CT26 cells were injected s.c. into Balb/C mice. Treatment of the mice was started when tumors reached approx. 100 mm³. Mice were injected at D0, D2 and D5 with COPTG19407 (8.5×10⁶ pfu i.t.), VVTG18058 (8.5×10⁶ pfu i.t.), m5-B07 (10 mg/kg i.p.) or the combination of VVTG18058 (8.5×10⁶ pfu i.t.) plus m5-B07 (10 mg/kg i.p.). FIG. 34A-D: Tumor growth and FIG. 34E: survival were followed over time.

FIG. 35: Individual tumor volume curves of BALB/c mice bearing subcutaneous A20 tumors.

A20 cells were injected s.c. into Balb/C mice. Treatment of the mice was started when tumors reached 80-100 mm³. Mice were injected at D0, D2 and D4 with vehicle (i.t), COPTG19407 (4.75×10⁶ pfu i.t.), VVTG18058 (4.75×10⁶ pfu i.t.), RMP1-14 (anti-mPD-1) (250 μg/mouse i.p.) or the combination of COPTG19407 (4.75×10⁶ pfu i.t.) plus RMP1-14 (250 μg/mouse i.p.).

A) Group 1 animals treated with vehicle

B) Group 2 animals treated with VVTG18058

C) Group 3 animals treated with COPTG19407

D) Group 4 animals treated with murine anti-PD1

E) Group 5 animals treated with COPTG19407 and murine anti-PD1

FIG. 36: Mean tumor volume curves of BALB/cN mice bearing subcutaneous A20 tumors.

Each point represents the mean of the recorded tumor volume per group. The tumor volumes of all animals were monitored over 64 days. Mice were treated with vehicle (group 1), VVTG18058 (group 2), COPTG19407 (group 3), the murine anti-PD1 antibody RMP1-14 (BioXCell) (group 4) and COPTG19407 and RMP1-14 (group 5). Animals were randomized on D7 and treated during the period D7 to D31. Last mice were sacrificed on D61.

FIG. 37: Antitumoral activity in A20 model: effect of COPTG19407+/−anti-PD1 on A20 tumor growth (A) and mice survival (B)

A20 cells were injected s.c. into Balb/C mice. Treatment of the mice was started when tumors reached 80-100 mm³. Mice were treated with vehicle (i.t.) (group 1), anti-PD-1 (group 2), isotype (group 3), VVTG18058 (10⁵ pfu i.t.) (group 4), VVTG18058 (10⁵ pfu i.t.)+isotype (group 5), VVTG18058 (10⁵ pfu i.t.)+anti-PD-1 (group 6), VVTG19407 (10⁵ pfu i.t.) (group 7), VVTG19407 (10⁵ pfu i.t.)+isotype (group 8) and VVTG19407 (10⁵ pfu i.t.)+anti-PD-1 (group 9).

FIG. 38: Individual tumor volume curves of C57BL/6 mice bearing subcutaneous C38 tumors.

C38 cells were injected s.c. into C57bl/6 mice. Treatment of the mice was started when tumors reached 80-100 mm³. Mice were injected at D0, D2 and D4 with vehicle (i.t), COPTG19407 (4.75×10⁶ pfu i.t.), VVTG18058 (4.75×10⁶ pfu i.t.), RMP1-14 (anti-mPD-1) (250 μg/mouse i.p.) or the combination of COPTG19407 (4.75×10⁶ pfu i.t.) plus RMP1-14 (250 μg/mouse i.p.).

A) Group 1 animals treated with vehicle

B) Group 2 animals treated with VVTG18058

C) Group 3 animals treated with COPTG19407

D) Group 4 animals treated with murine anti-PD1

E) Group 5 animals treated with COPTG19407 and murine anti-PD1

FIG. 39: Mean tumor volume curves of C57BL/6 mice bearing subcutaneous C38 tumors.

Each point represents the mean of the recorded tumor volume per group. The tumor volumes of all animals were monitored over 61 days. Mice were treated with vehicle (group 1), VVTG18058 (group 2), COPTG19407 (group 3), the murine anti-PD1 antibody RMP1-14 (BioXCell) (group 4) and COPTG19407 and RMP1-14 (group 5). Animals were randomized on D7 and treated during the period D7 to D31. Last mice were sacrificed on D61.

FIG. 40: Individual tumor volume curves of BALB/c mice bearing subcutaneous EMT6 tumors.

EMT6 cells were injected s.c. into Balb/C mice. Treatment of the mice was started when tumors reached 80-100 mm³. Mice were injected at D0, D2 and D4 with vehicle (i.t), COPTG19407 (4.75×10⁶ pfu i.t.), VVTG18058 (4.75×10⁶ pfu i.t.), RMP1-14 (anti-mPD-1) (250 μg/mouse i.p.) or the combination of COPTG19407 (4.75×10⁶ pfu i.t.) plus RMP1-14 (250 μg/mouse i.p.).

A) Group 1 animals treated with vehicle

B) Group 2 animals treated with VVTG18058

C) Group 3 animals treated with COPTG19407

D) Group 4 animals treated with murine anti-PD1

E) Group 5 animals treated with COPTG19407 and murine anti-PD1

FIG. 41: Mean tumor volume curves of BALB/c mice bearing subcutaneous EMT6 tumors.

Each point represents the mean of the recorded tumor volume per group. The tumor volumes of all animals were monitored over 61 days. Mice were treated with vehicle (group 1), VVTG18058 (group 2), COPTG19407 (group 3), the murine anti-PD1 antibody RMP1-14 (BioXCell) (group 4) and COPTG19407 and RMP1-14 (group 5). Animals were randomized on D7 and treated during the period D7 to D31. Last mice were sacrificed on D56. Curves were stopped after the death of more than 20% of mice.

### EXAMPLES

Specific, non-limiting examples which embody certain aspects of the invention will now be described. In the examples, rh protein denotes a human recombinant protein (e.g. rhIL-2 denotes human recombinant IL-2 protein) and rcm protein denotes a cynomologous recombinant protein (e.g. rcmCTLA4 denotes cynomologous recombinant CTLA-4 protein).

In addition to sequences mentioned above, some additional sequences are used in the examples, and these are set ut in Table 5 below.

TABLE 5
Additional sequences used in the examples

| | | SEQ. |
|---|---|---|
| | Sequence | ID. NO |
| Human | APARSPSPSTQPWEHVNAIQEARRLLNLSRDT | 55 |
| GM-CSF | AAEMNETVEVIEMFDLQEPTCLQTRLELYKQG | |
| without | LRGSLTKLKGPLTMMASHYKQHCPPTPETSCA | |
| signal | TQTITFESFKENLKDFLLVIPFDCWEPVQE | |
| peptide | | |
| Human | MWLQSLLLLGTVACSISAPARSPSPSTQPWEH | 56 |
| GM-CSF | VNAIQEARRLLNLSRDTAAEMNETVEVISEMF | |
| with | DLQEPTCLQTRLELYKQGLRGSLTKLKGPLTM | |
| signal | MASHYKQHCPPTPETSCATQTITFESFKENLK | |
| peptide | DFLLVIPFDCWEPVQE | |
| Murine | APTRSPITVTRPWKHVEAIKEALNLLDDMPVT | 57 |
| GM-CSF | LNEEVEVVSNEFSFKKLTCVQTRLKIFEQGLR | |
| without | GNFTKLKGALNMTASYYQTYCPPTPETDCETQ | |
| signal | VTTYADFIDSLKTFLTDIPFECKKPVQK | |
| peptide | | |
| Murine | MWLQNLLFLGIVVYSLSAPTRSPITVTRPWKH | 58 |
| GM-CSF | VEAIKEALNLLDDMPVTLNEEVEVVSNEFSFK | |
| with | KLTCVQTRLKIFEQGLRGNFTKLKGALNMTAS | |
| signal | YYQTYCPPTPETDCETQVTTYADFIDSLKTFL | |
| peptide | TDIPFECKKPVQK | |
| Promoter | CCACCCACTTTTTATAGTAAGTTTTTCACCCA | 59 |
| p7.5K | TAAATAATAAATACAATAATTAATTTCTCGTA | |
| | AAAGTAGAAAATATATTCTAATTTATTGCACG | |
| | GTAAGGAAGTAGAATCATAAAGAACAGT | |
| Promoter | TTTATTCTATACTTAAAAAATGAAAATAAATA | 60 |
| pH5.4 | CAAAGGTTCTTGAGGGTTGTGTTAAATTGAAA | |
| | GCGAGAAATAATCATAAATTATTTCATTATCG | |
| | CGATATCCGTTAAGTTTG | |
| Promoter | AAAAATTGAAATTTTATTTTTTTTTTTTGGAA | 61 |
| pSE/L | TATAAATA | |

### Example 1-Generation of CTLA-4 Specific Antibodies

Isolation of scFv Antibody Fragments

The n-CoDeR® scFv library (BioInvent; Soderlind E, et al Nat Biotechnol. 2000; 18(8):852-6) was used to isolate scFv antibody fragments recognizing human CTLA-4.

The phage library was used in three consecutive pannings against recombinant human protein. After phage incubation, the cells were washed to remove unbound phages. Binding phages were eluted with trypsin and amplified in E. coli. The resulting phage stock was converted to scFv format. E. coli was transformed with scFv bearing plasmids and individual scFv clones were expressed.

Identification of Unique CTLA-4 Binding scFv

Converted scFv from the third panning were assayed using a homogeneous FMAT analysis (Applied Biosystems, Carlsbad, CA, USA) for binding to transfected 293 FT cells expressing human CTLA-4 or a non-target protein.

Briefly, transfected cells were added to clear-bottom plates, together with the scFv-containing supernatant from expression plates (diluted 1:7), mouse anti-His Tag antibody (0.4 μg/ml; R&D Systems) and an APC-conjugated goat anti-mouse antibody (0.2 μg/ml; cat. no. 115-136-146, Jackson Immunoresearch). FMAT plates were incubated at room temperature (approximately 20-25° C.) for 9 h prior to reading. Target-specific bacterial clones were classified as actives and cherry picked into 96-well plate.

### IgG Binding to CTLA-4 in ELISA

96-well plates (Lumitrac 600 LIA plate, Greiner) were coated overnight at 4° C. with recombinant human CTLA-4-Fc protein (R&D Systems) at 1 pmol/well, recombinant cynomologous (cm) CTLA-4-Fc protein (R&D Systems) at 1 pmol/well, recombinant mouse CTLA-4-Fc protein (R&D Systems) at 0.3 pmol/well, recombinant human CD28-Fc protein (R&D Systems) at 1 pmol/well or recombinant mouse CD28-Fc protein (R&D Systems) at 0.3 pmol/well. After washing, titrated doses of anti-CTLA-4 mAbs from 0 μg/ml to 0.06 ng/ml (66 nM to 0.3 pM) were allowed to bind for 1 hour. Plates were then washed again and bound antibodies were detected with an anti-human-F(ab)-HRP secondary antibody (Jackson ImmunoResearch) diluted in 50 ng/ml. Super Signal ELISA Pico (Thermo Scientific) was used as substrate and the plates were analysed using Tecan Ultra Microplate reader.

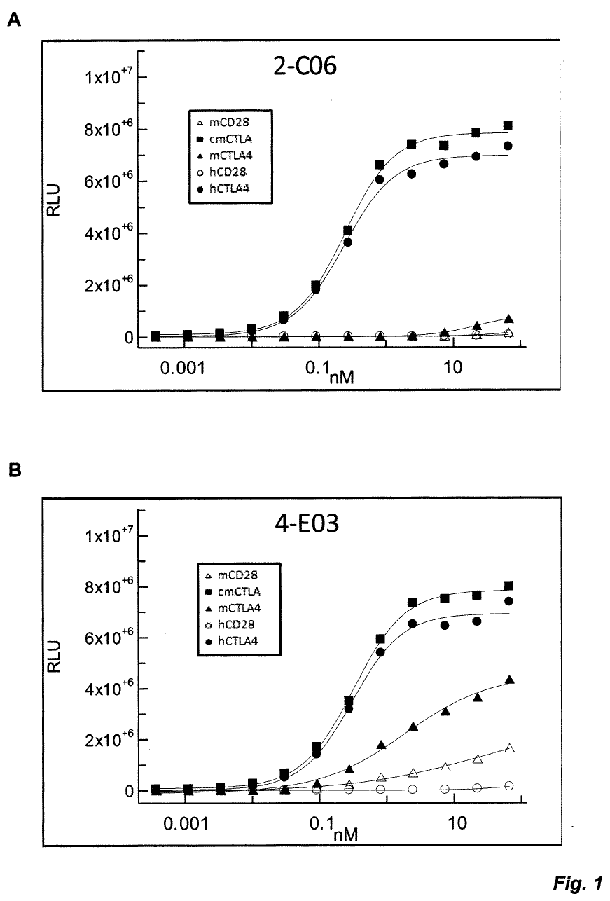

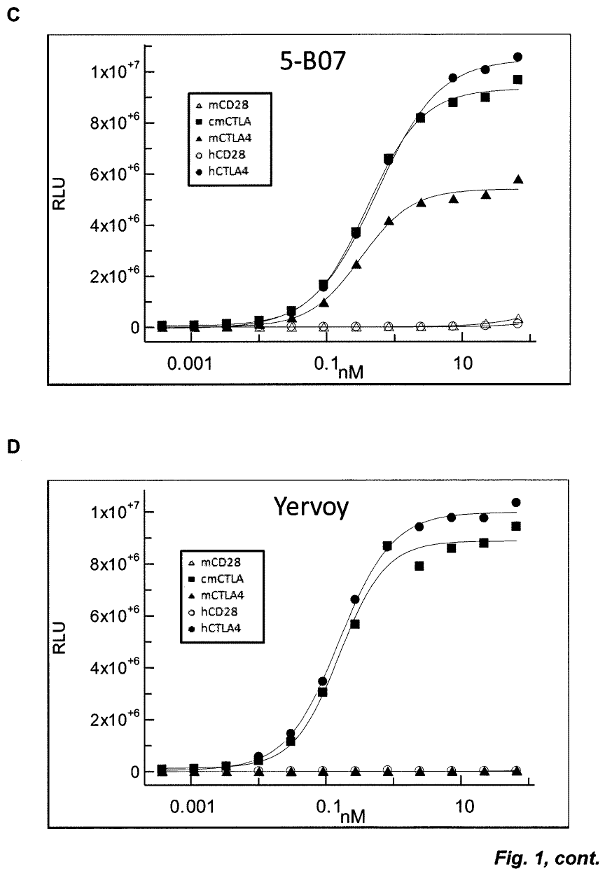

All antibodies were shown to bind to human and cynomologous CTLA-4 protein but not to human CD28 protein as assayed by ELISA. In addition, 5-B07 was shown to bind to mouse CTLA-4 but not mouse CD28 (see FIG. 1).

### IgG Binding to CTLA-4-Expressing 293T Cells in Flow Cytometry

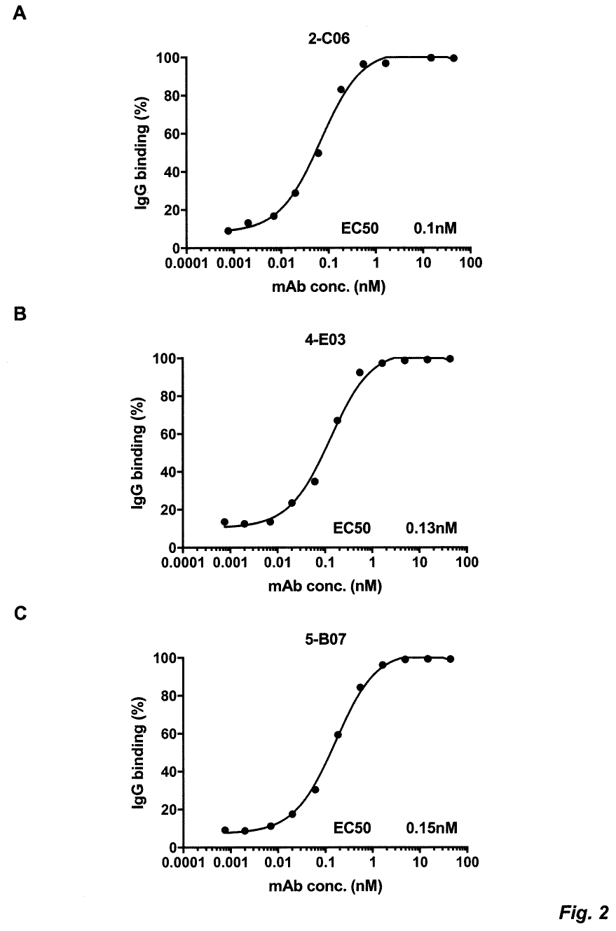

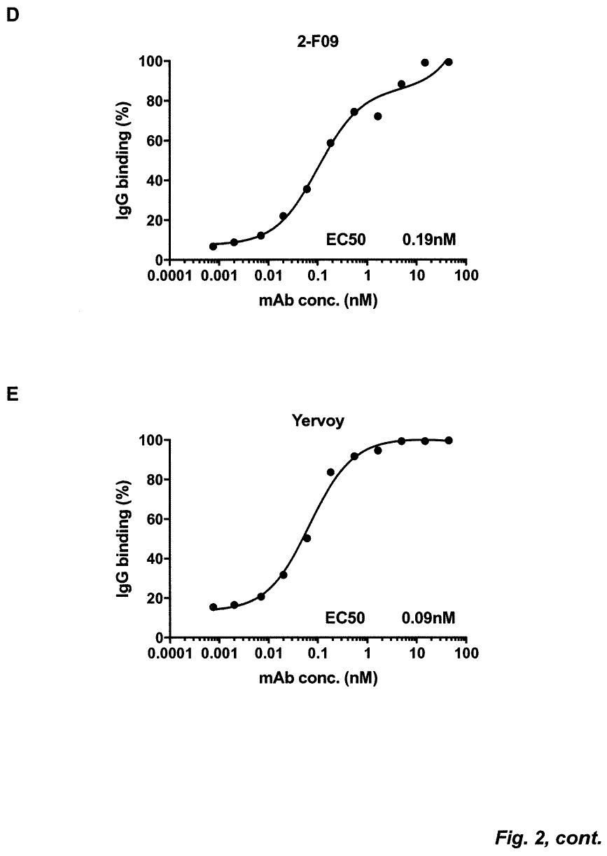

Converted IgG clones were analysed for binding to CTLA-4-expressing 293T cells (purchased from Crownbio). Cells were incubated with different concentrations (as indicated in FIG. 2) of anti-CTLA-4 mAb at 4° C. for 20 min prior to washing and staining with an APC-labelled goat anti-human secondary antibody (cat. no. 109-136-088, Jackson ImmunoResearch). Dead cells were excluded from analyses using Fixable Viability Dye eFluor780 (eBiosciences). Data acquisition was performed on a FACSVerse (BD Biosciences, Franklin Lakes, NJ) and analysed with FlowJo software (Tree Star, Ashland, OR).

The anti-CTLA-4 mAbs were shown to bind human CTLA-4-expressing 293T cells in a dose-dependent manner with a similar EC50 value as Yervoy (FIG. 2). 293T cells stably transfected with human CTLA-4, 293T cells transiently transfected with cynomologous CTLA-4, naive human or cynomologous PBMCs, in vitro-activated human or cyno CD4+ T cells were incubated with the concentrations of anti-CTLA-4 mAb indicated at 4° C. for 20 min prior to washing and staining with a APC-labelled anti-human secondary antibody (Jackson ImmunoResearch).

### Example 2-Anti-CTLA-4 mAb Specifically Bind Human and Cynomologous CTLA-4-Expressing (Primary) Cells

CTLA-4 Specific mAb Bind Primary Human and Cynomologous In Vitro-Activated CD4+ T Cells but not Naïve PBMCs Isolated from Healthy Donors

PBMCs were isolated from buffy coats. Briefly, buffy were diluted 1:3 in PBS and were loaded onto Ficoll-Paque Plus (Amersham) cushions. Samples were centrifuged at 800×g for 20 min at 20° C. The upper, plasma-containing phase was removed and mononuclear cells were isolated from the distinct white band at the plasma/Ficoll interphase.

Human peripheral CD4+ T-cells were purified by negative selection using MACS CD4 T-cell isolation kit (Miltenyi Biotec). CD4+ T cells were activated in vitro with CD3/CD28 dynabeads (Life Technologies) plus 50 ng/ml rhIL-2 (R&D Systems) in R10 medium (RPMI containing 2 mM glutamine, 1 mM pyruvate, 100 IU/ml penicillin and streptomycin and 10% FBS (GIBCO by Life Technologies) for 3 days to upregulate CTLA-4 expression. Cynomologous CD4+ T cells were isolated using non-human CD4 microbeads (Miltenyi Biotec) and incubated with 50 ng/ml PMA (Sigma-Aldrich) and 100 ng/ml lonomycin (Sigma-Aldrich) for 3 days.

Naive human or cynomologous PBMCs, in vitro-activated human or cyno CD4+ T cells were incubated with the indicated concentrations of anti-CTLA-4 mAb at 4° C. for 20 min prior to washing and staining with a APC-labelled anti-human secondary antibody (Jackson ImmunoResearch). Binding of anti-CTLA-4 mAb was analysed by FACS using a BD FACS Verse.

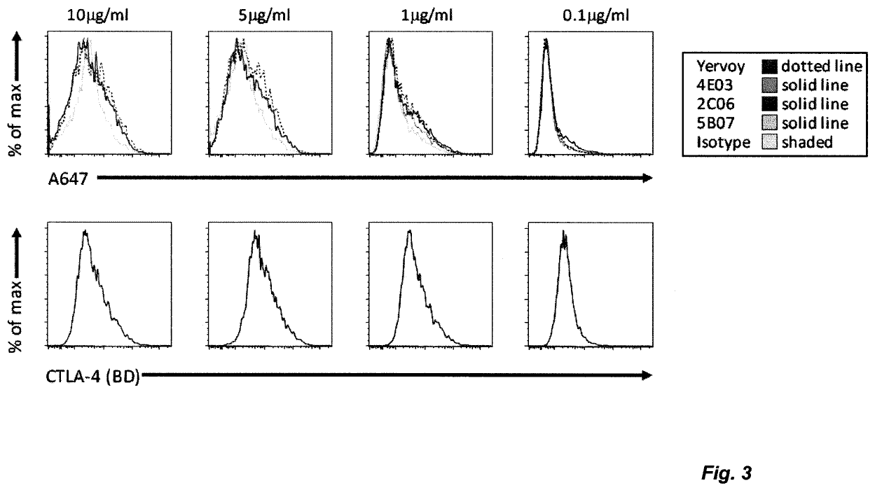

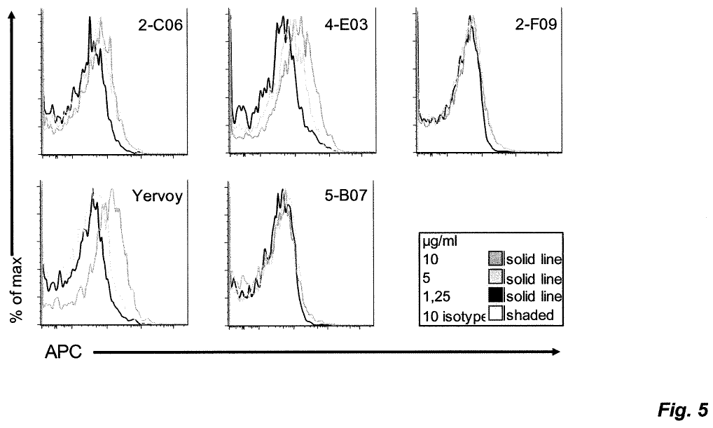

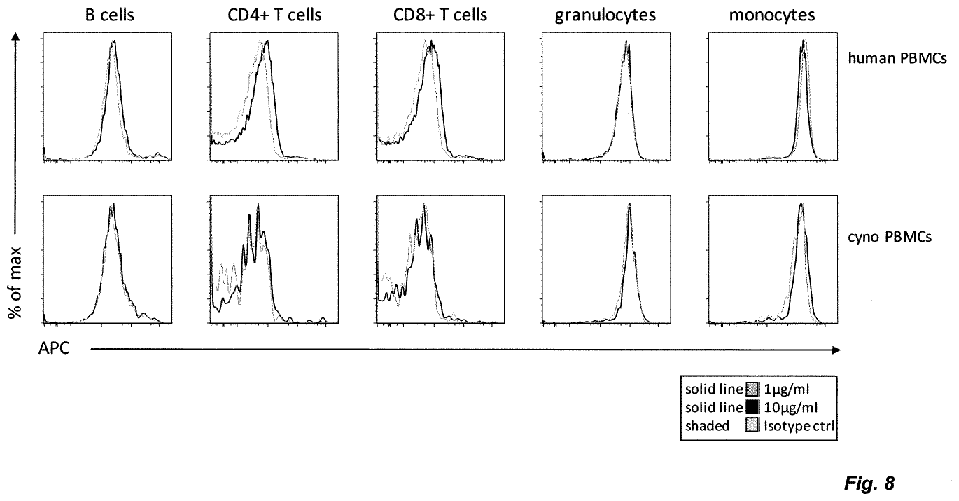

The antibodies were shown to bind to in vitro-activated human (FIG. 3) and cynomologous (FIG. 5) CD4+ T cells but not to resting PBMCs (FIG. 8). The binding to endogenously CTLA-4 expressing T cells is similar to the staining with Yervoy (FIG. 3, upper row, dotted line) and a as a positive control a commercial anti-CTLA-4 FACS-antibody from BD Biosciences (clone BNI3; FIG. 3, bottom row).

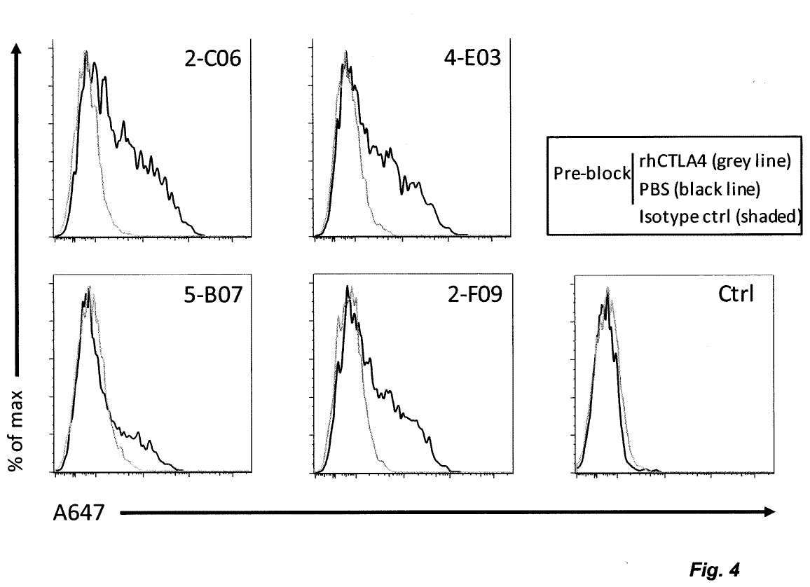

As shown in FIG. 4, the staining of human in vitro-activated CD4+ T cells (black line) can be completely blocked by rhCTLA-4-Fc (grey line) demonstrating the specificity of the antibodies. In this competitive binding assay, 2 μg/ml Alexa 647-labelled anti-CTLA-4 mAb was mixed with recombinant human CTLA-4-Fc protein (50 μg/ml) prior to incubation with CTLA-4 expressing cells. IgG binding was detected by FACS.

### Transfected 293T Cells Expressing Human and Cynomologous CTLA-4 Confirm Cynocrossreactivity of the Tested Antibodies

The cyno-crossreactivity of the antibodies was further confirmed on transfected CTLA-4 expressing 293T cells.

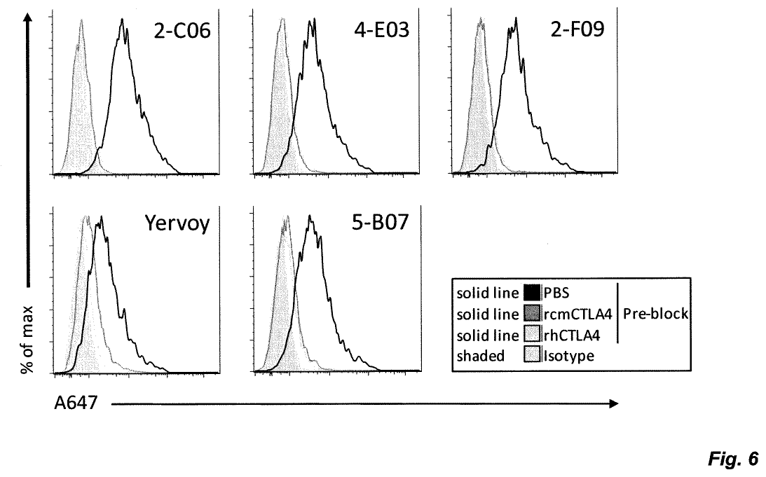

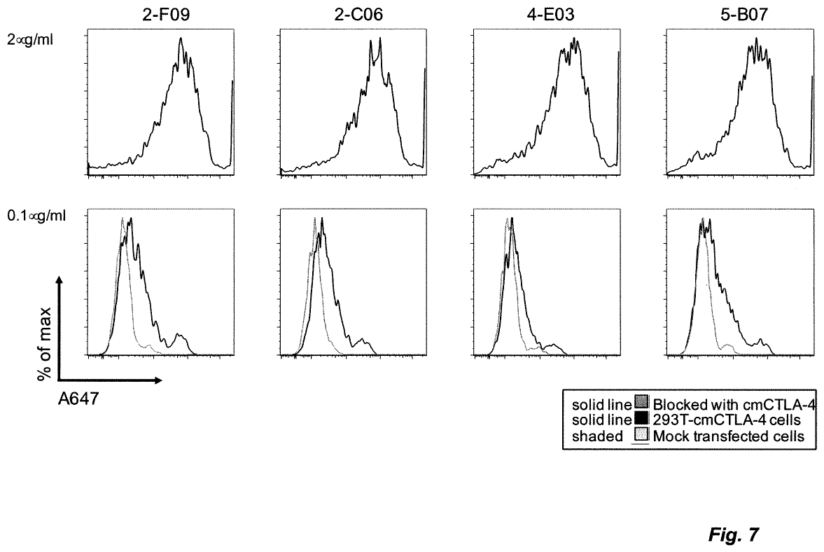

As demonstrated in FIG. 6, the binding of CTLA-4 specific antibodies to human CTLA-4 expressing transfected cells can be inhibited by human and cynomologous recombinant protein (both R&D Systems). The antibodies were also shown to bind to transfected cells expressing cynomologous CTLA-4 (FIG. 7, upper row). This binding can be blocked again by cynomologous recombinant protein (bottom row, grey line). The experiments were performed similar to the competitive assay described above in Example 2 in connection with FIG. 4.

### Expected Lack of Direct Agonistic Activity

In vitro-proliferation assays were performed to exclude unanticipated direct agonistic activity (e.g. due to unspecific binding).

Human peripheral CD4+ T-cells were purified from healthy PBMCs by negative selection using MACS CD4 T-cell isolation kit (Miltenyi Biotec) and were thereafter labelled with CFSE (2 μM, Molecular Probes). Antibodies were cross-linked with F(ab′)₂ goat anti-human IgG, Fcγ fragment specific or F(ab′)₂ goat anti-mouse IgG, Fcγ fragment specific in a molar ratio IgG:F(ab′)₂=1.5:1 for 1 h at RT. 1×10⁵ purified human CD4+ T cells were stimulated with plate-bound anti-CD3 (0.5 μg/ml; clone UCHT1, R&D Systems) and 4 μg/ml of soluble, cross-linked anti-CTLA-4 or crosslinked anti-CD28 (clone CD28.2, BioLegend) for 72 hours at 37° C. Cells were washed and stained with a BV421-conjugated anti-CD25 antibody (clone M-A251, BD Biosciences). The percentage of CD25+/CFSElow dividing cells was analysed by FACS.

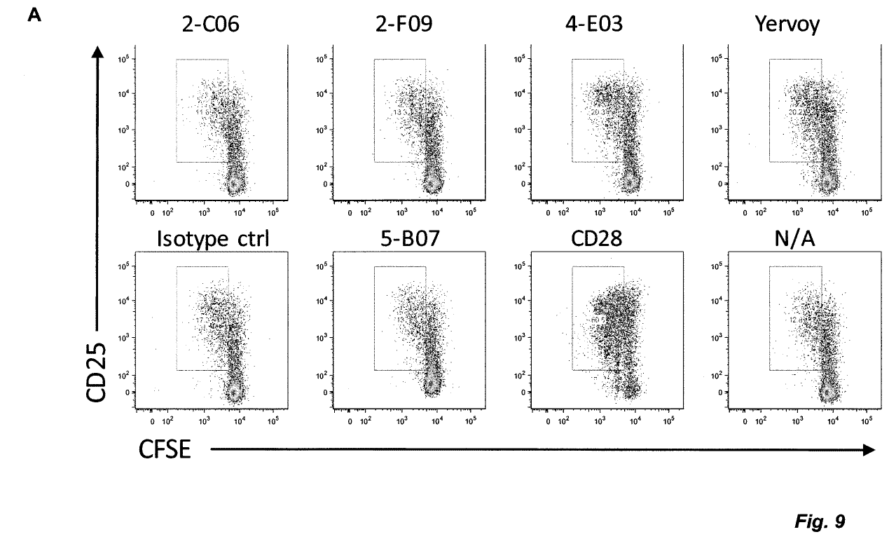

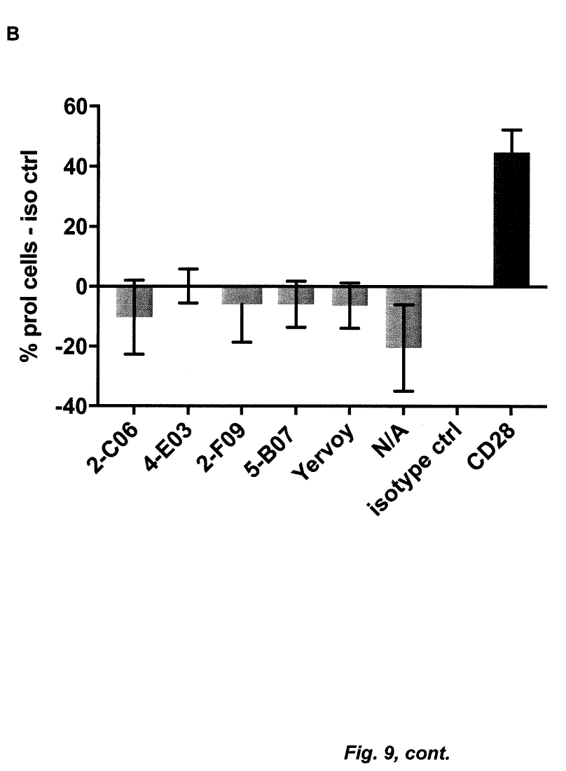

FIG. 9 demonstrates that none of the tested anti-CTLA-4 mAb induces T cell proliferation in contrast to anti-CD28 stimulation.

### Example 3-Anti-CTLA-4 mAb Block Ligand Binding of CD80/CD86 Ligand Blocking ELISA

The ligand blocking activity of anti-CTLA-4 IgGs was assessed by ELISA. To this end, recombinant human CTLA-4-Fc protein (R&D Systems) was coated to 96-well plates (Lumitrac 600 LIA plate, Greiner) at 1 pmol/well. After washing, titrated doses of anti-CTLA-4 mAbs were allowed to bind for 1 hour. His-tagged ligands were added at 200 nM and 100 nM, respectively (rhCD80 and rhCD86; R&D Systems) and the plates were further incubated for 15 minutes. After washing, bound ligand was detected with an HRP-labelled anti-His antibody (R&D Systems). Super Signal ELISA Pico (Thermo Scientific) was used as substrate and the plates were analysed using Tecan Ultra Microplate reader.

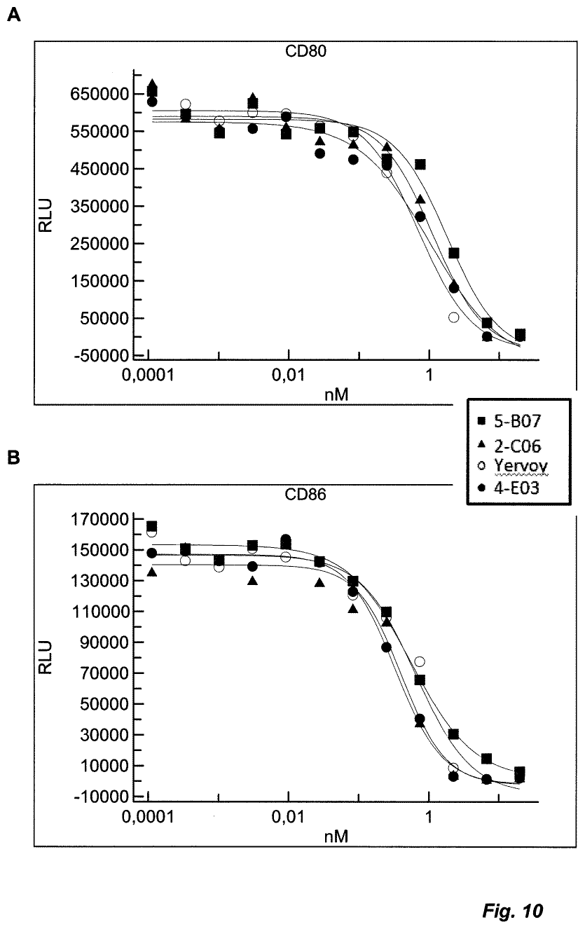

As shown in FIG. 10, anti-CTLA-4 antibodies tested show similar ligand blocking activity as Yervoy.

### Functional Ligand Block In Vitro

For the SEB PBMC assay, total PBMCs from healthy donors were seeded on 96-well plates (1×10⁵ cells/well) and stimulated with 1 g/ml Staphylococcus enterotoxin B (SEB, Sigma Aldrich) in the presence of titrated doses of anti-CTLA-4 IgGs. IL-2 secretion was measured by MSD (Mesoscale) on day 3 according to manufactures' instructions.

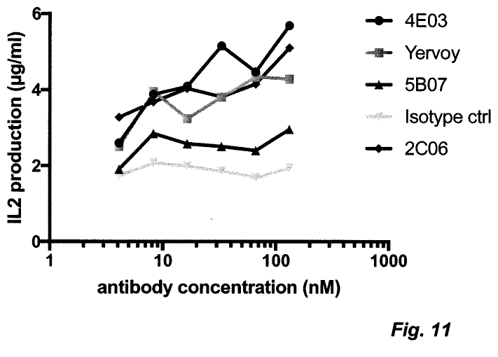

The antibodies 4-E03 and 2-C06 were shown to enhance IL-2 production and their potency was shown to be similar to that of Yervoy. In FIG. 11, one representative donor out of 6 is shown.

### Example 4-Anti-CTLA-4 mAb Deplete CTLA-4 Expressing Cells In Vitro and In Vivo Antibody Dependent Cellular Cytotoxicity (ADCC)

ADCC assays were performed using an NK-92 cell line stably transfected to express the CD16-158V allele together with GFP (purchased from Conkwest, San Diego, CA; Binyamin, L., et al., 2008, Blocking NK cell inhibitory self-recognition promotes antibody-dependent cellular cytotoxicity in a model of anti-lymphoma therapy. Journal of immunology 180, 6392-6401). CD4+ target T cells were isolated from peripheral blood of healthy donors using CD4+ T cell isolation kit (Miltenyi Biotec). Cells were stimulated for 48 hours with CD3/CD28 dynabeads (Life Technologies, Thermo Fisher) and 50 ng/ml rhIL-2 (R&D Systems) at 37° C. Target cells were pre-incubated with mAb at 10 μg/ml for 30 min at 4° C. prior to mixing with NK cells. The cells were incubated for 4h in RPMI 1640+GlutaMAX medium (Invitrogen) containing 10 mM HEPES buffer, 1 mM sodium Pyruvate and 10% FBS low IgG at a 2:1 effector:target cell ratio. Lysis was determined by flow cytometry. Briefly, at the end of the incubation, the cell suspension was stained with BV510-conjugated anti-CD4 (clone RPA-T4, BD Biosciences) together with 10 nM SYTOX Red dead cell stain (Invitrogen) or Fixable Viability Dye eFluor780 (eBioscience) for 20 min in the dark at 4° C. and the cells were then analysed using a FACSVerse (BD Biosciences).

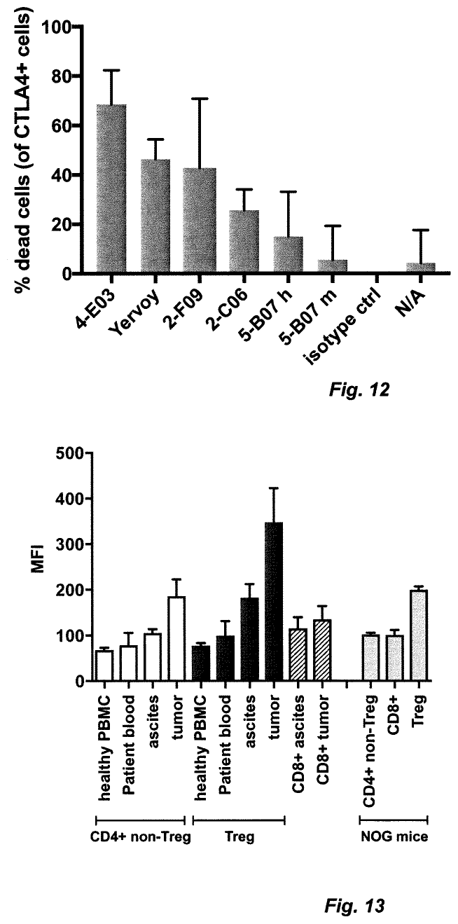

4-E03 showed a significantly improved deletion of CTLA-4+ T cells in vitro compared to Yervoy (FIG. 12).

### CTLA-4 Expression on Primary Patient Material

In order to validate the translational potential of the finding above on the depleting activity of anti-CTLA-4 mAb, the CTLA-4 expression was examined on primary patient material.

Ethical approval for the use of clinical samples was obtained by the Ethics Committee of Skåne University Hospital. Informed consent was provided in accordance with the Declaration of Helsinki. Samples were obtained through the Department of Gynocology and Department of Oncology at, Skånes University Hospital, Lund. Ascitic fluid was assessed as single cell suspensions that had been isolated. Tumour material was cut into small pieces and incubated in R10 with DNase I (Sigma Aldrich) and Liberase™ (Roche Diagnostics) for 20 min at 37° C. Remaining tissue was mechanically crashed and, together with the cell suspension, passed through a 70 μm cell strainer. Cells isolated from ascitic fluid and tumours were stained. To identify different T cell subsets following antibodies were used: CD4-BV510 (RPA-T4), CD25-BV421 (M-A251), anti-CD127-FITC (HIL-7R-M21), CTLA-4-PE (BNI3), CD8-PeCy7 (RPA-T8), CD3-APC (UCHT1), CD45-PercP-Cy5.5 (HI30), mouse IgG2a isotype, k control-PE (G155-178; all from BD Biosciences). Data acquisition was performed using FACSVerse and data analysed using FlowJo.

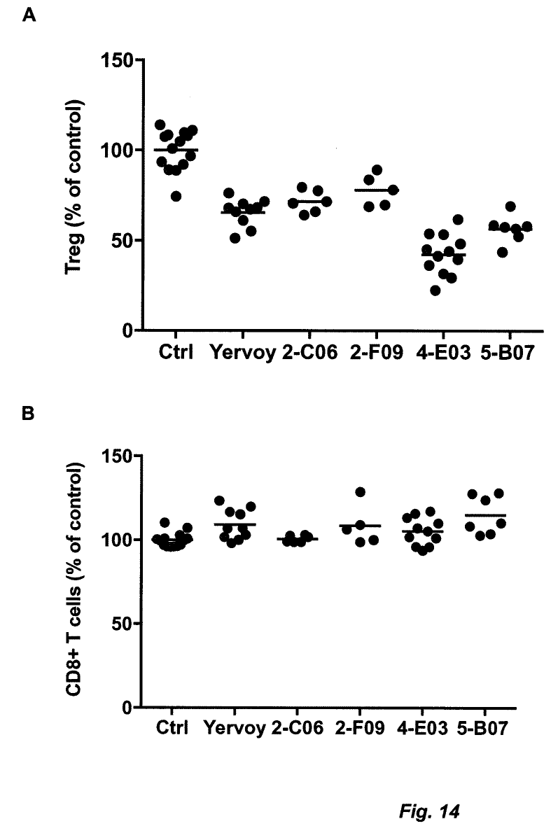

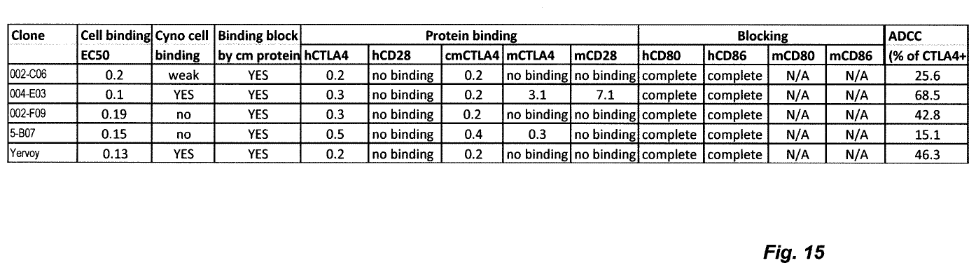

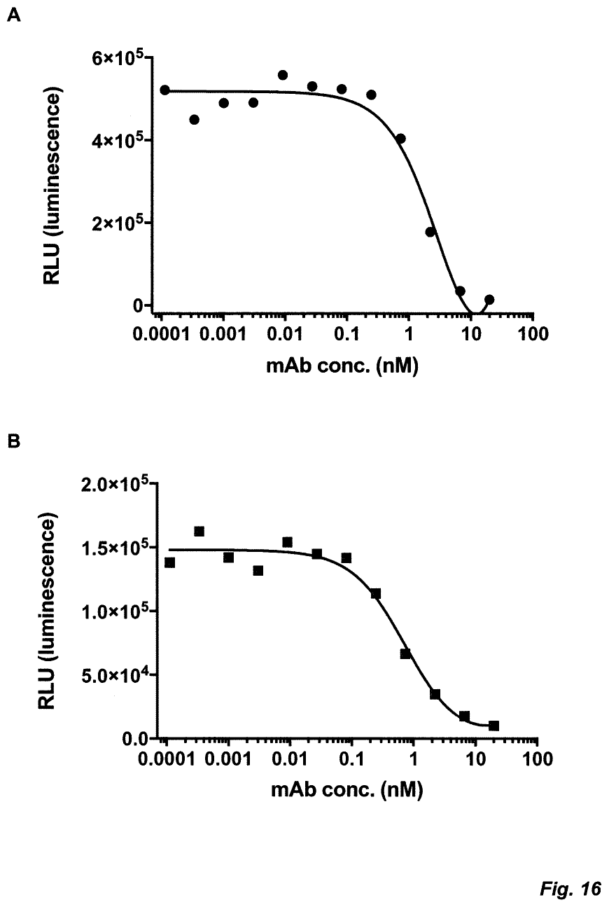

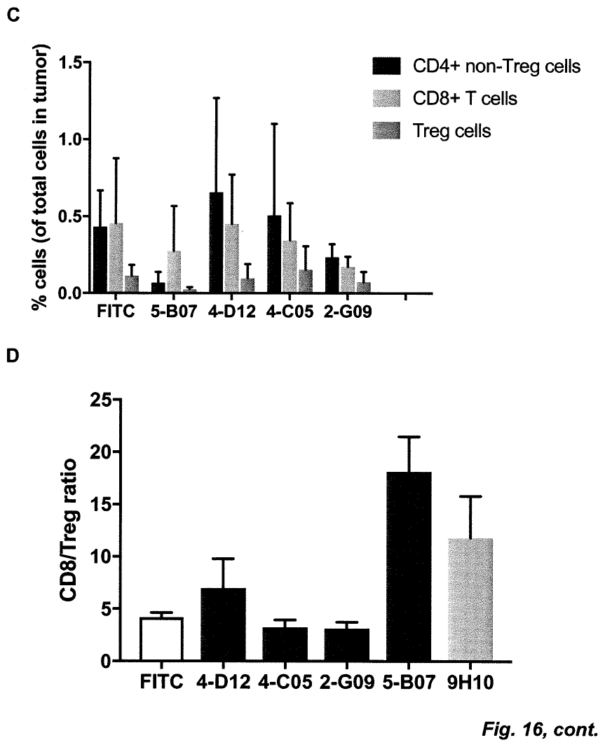

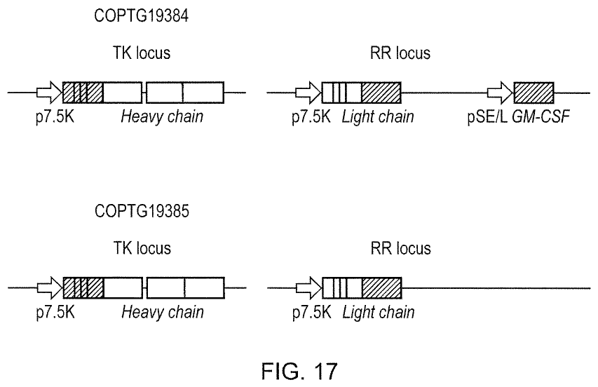

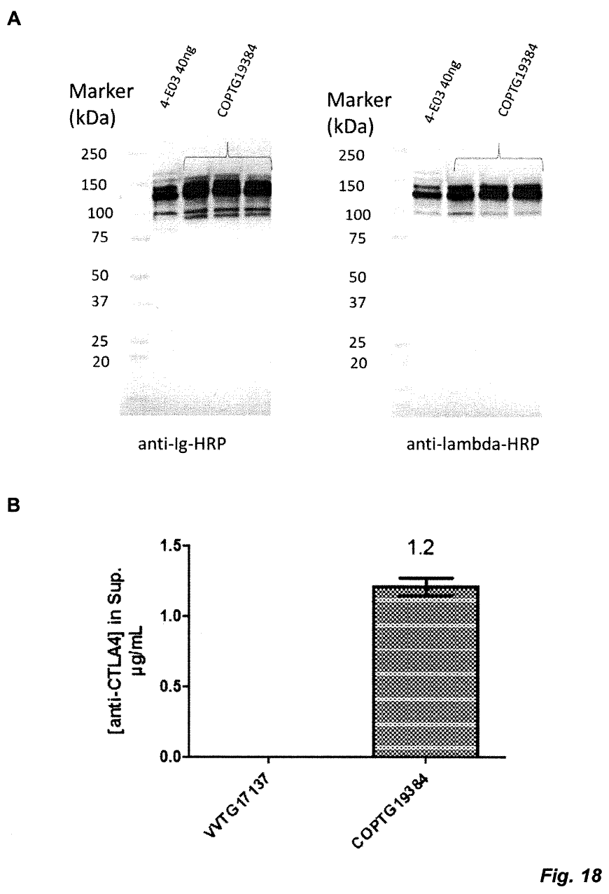

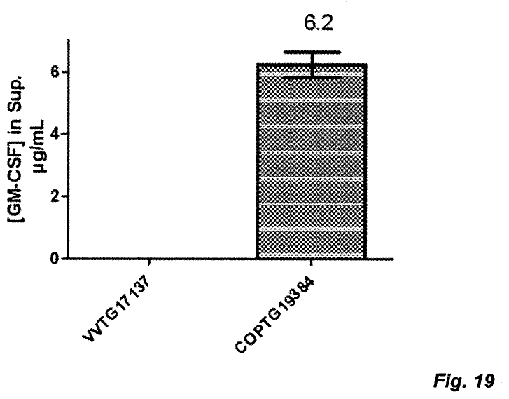

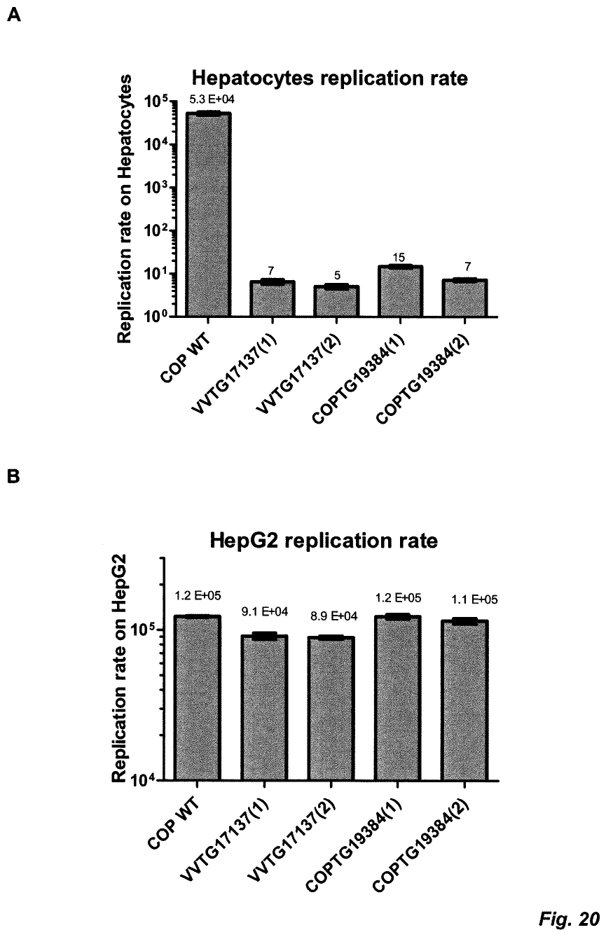

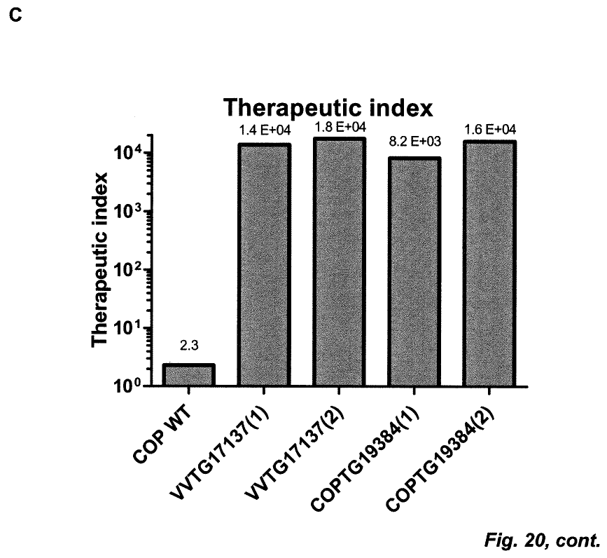

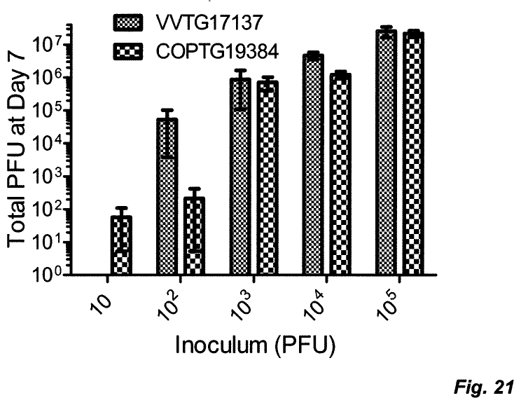

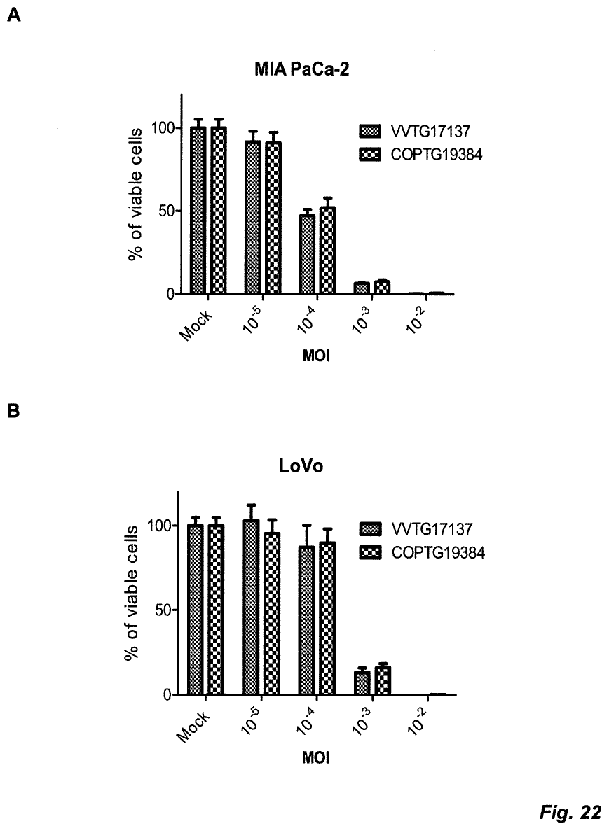

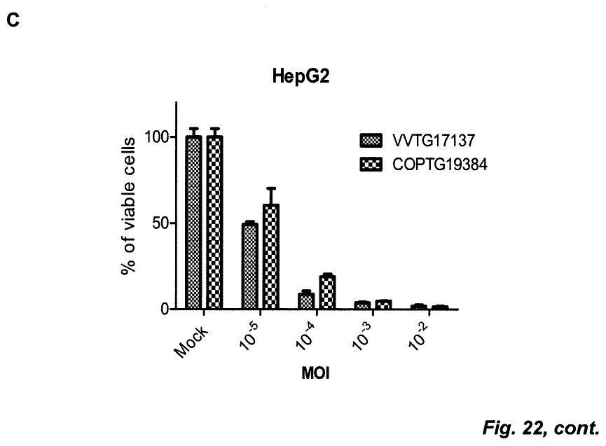

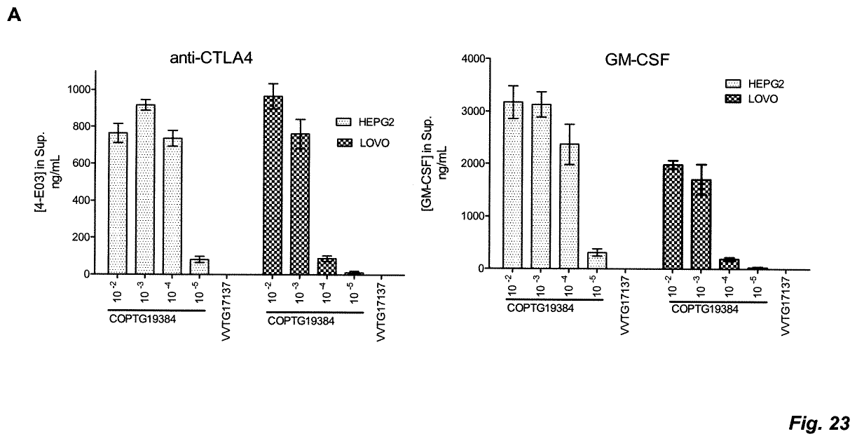

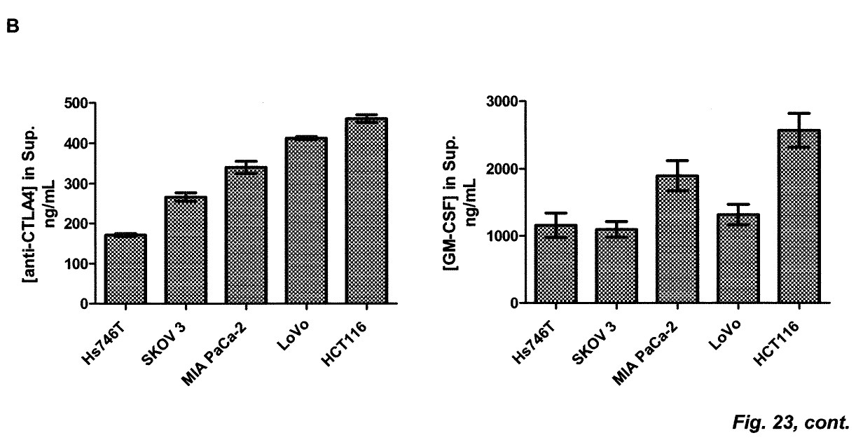

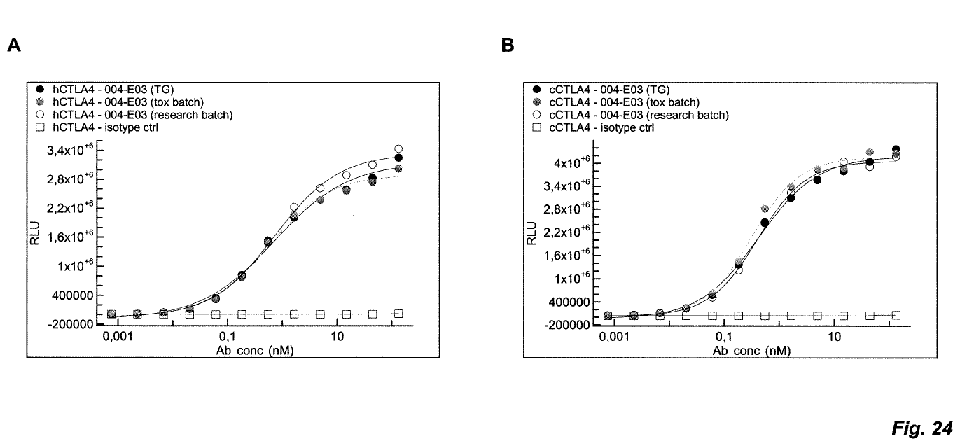

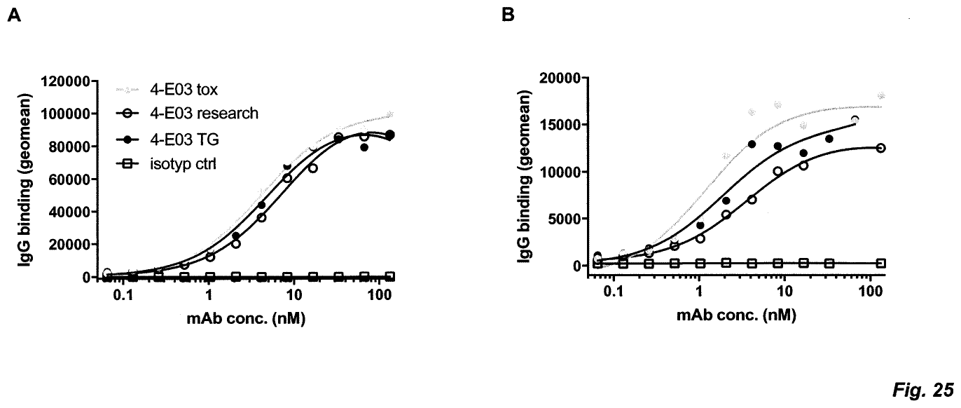

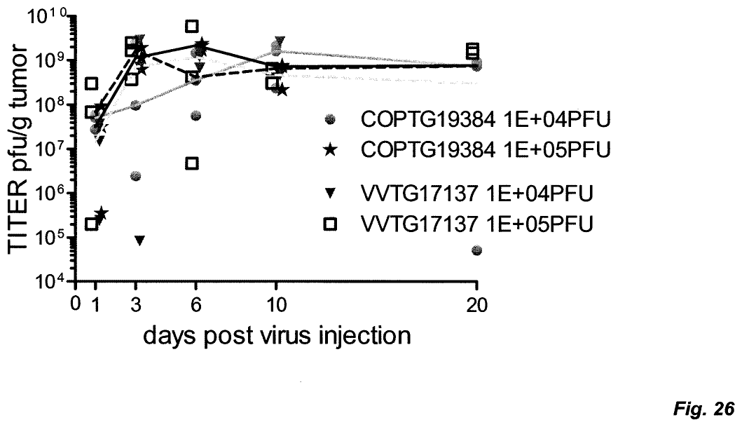

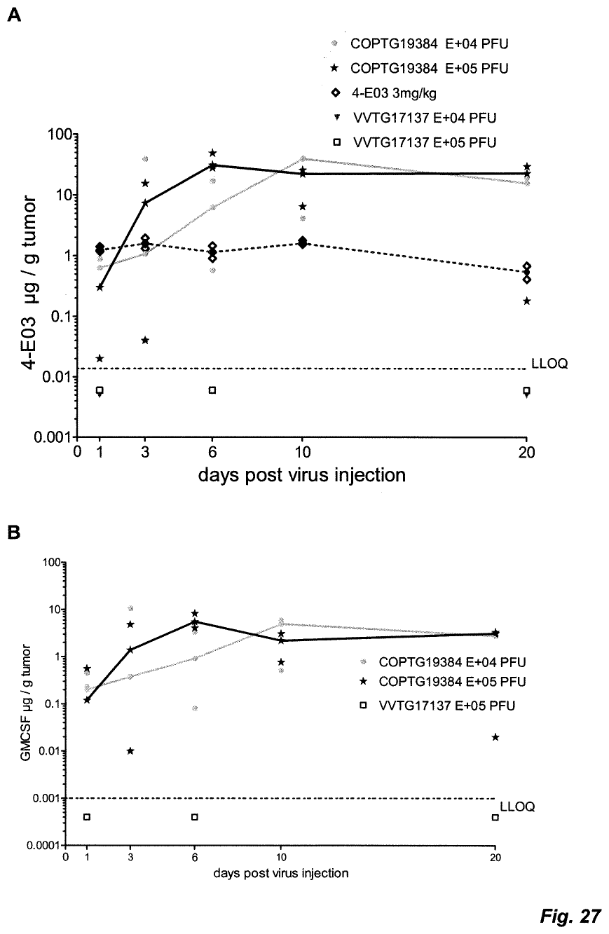

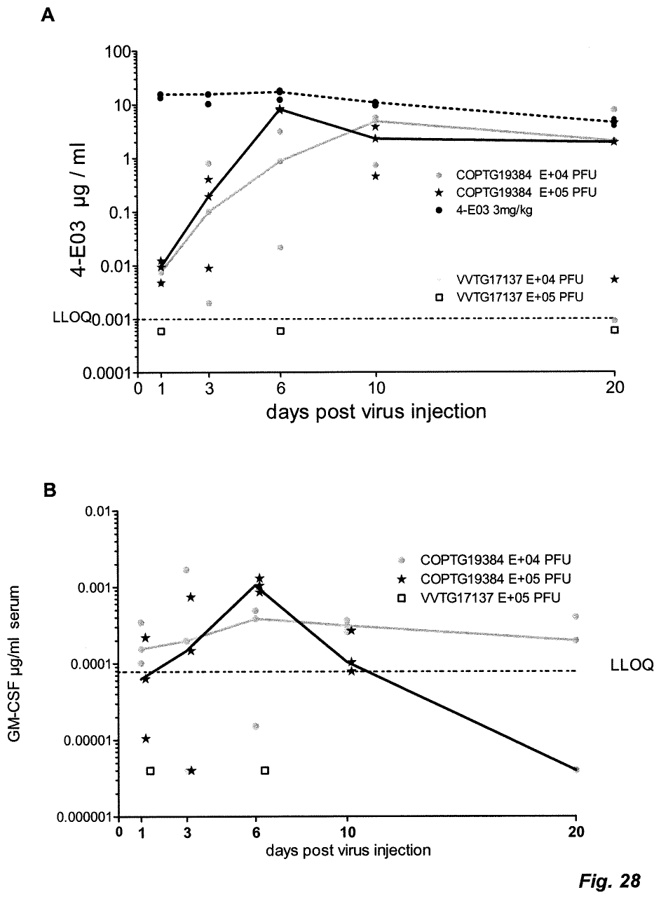

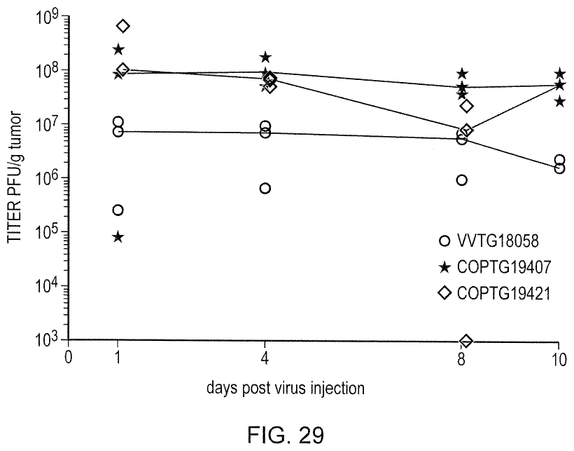

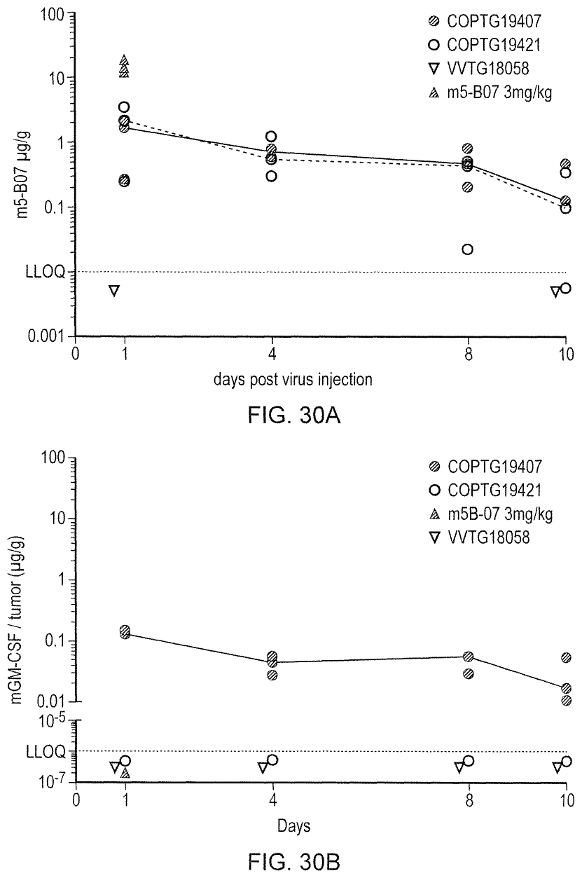

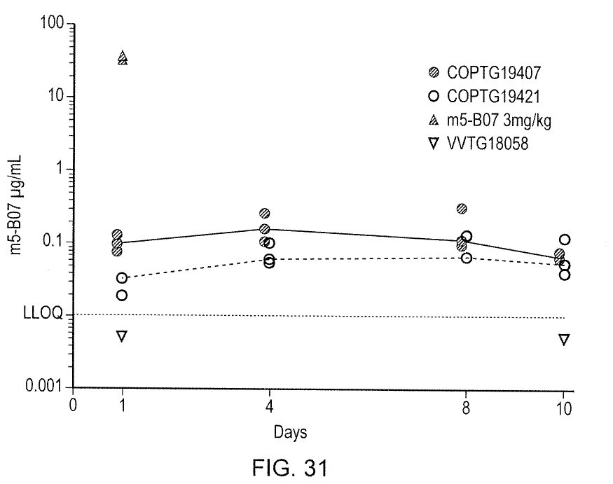

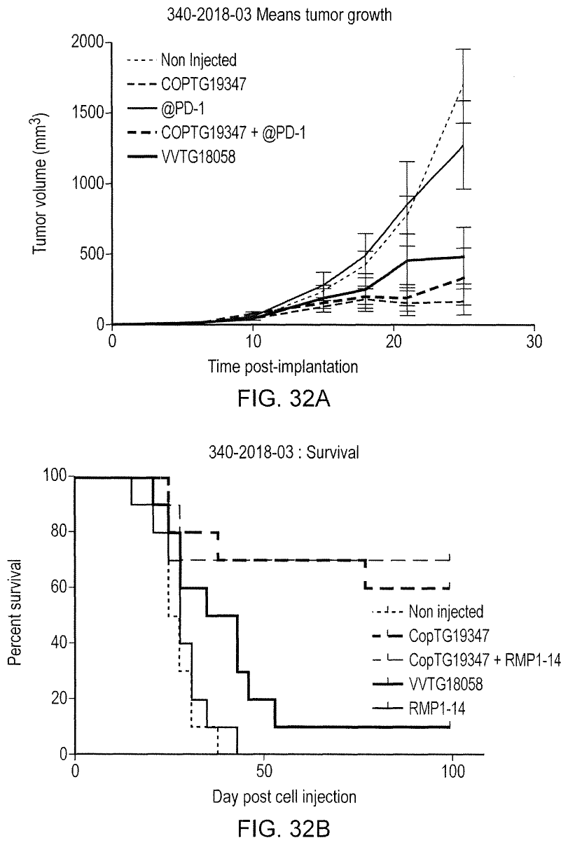

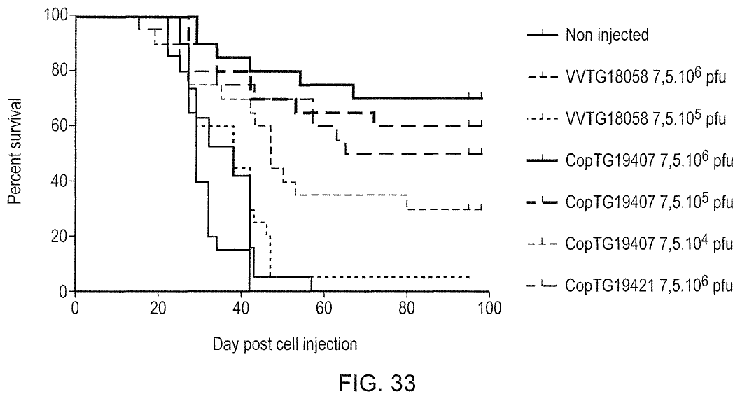

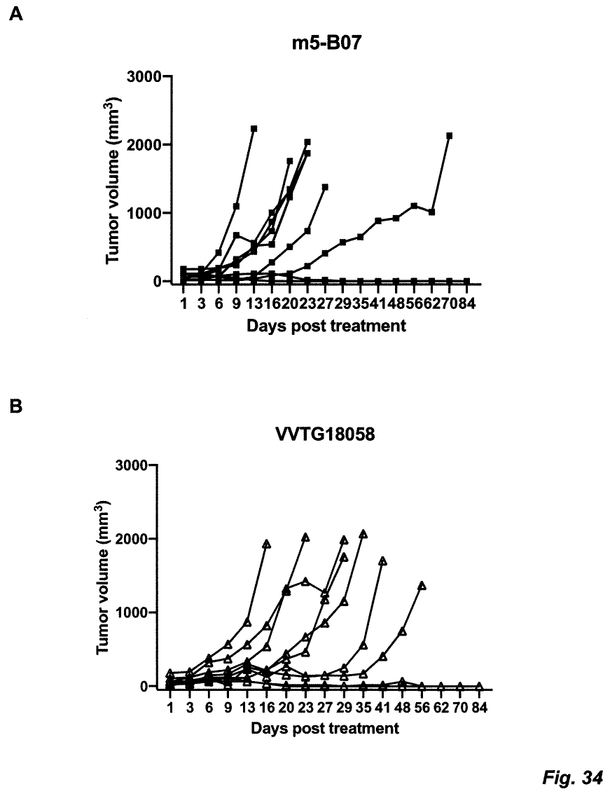

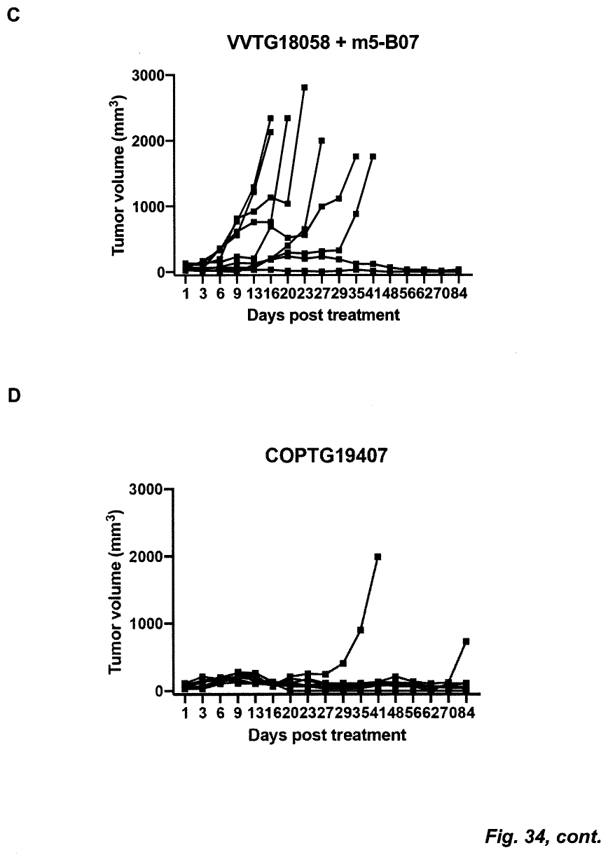

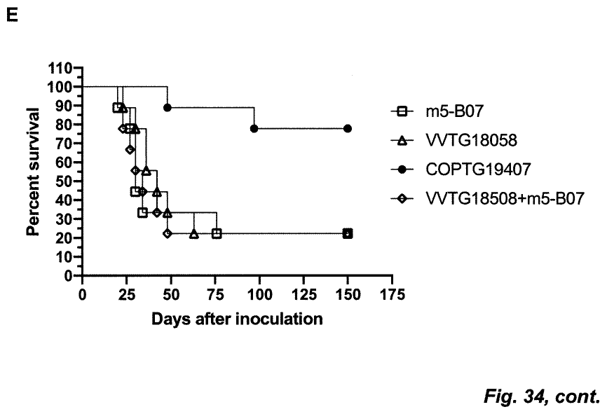

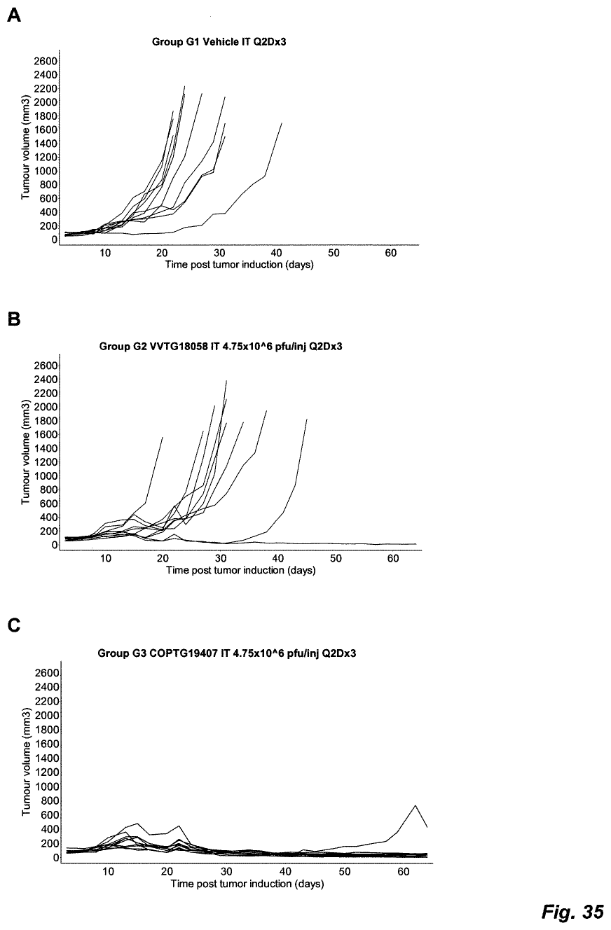

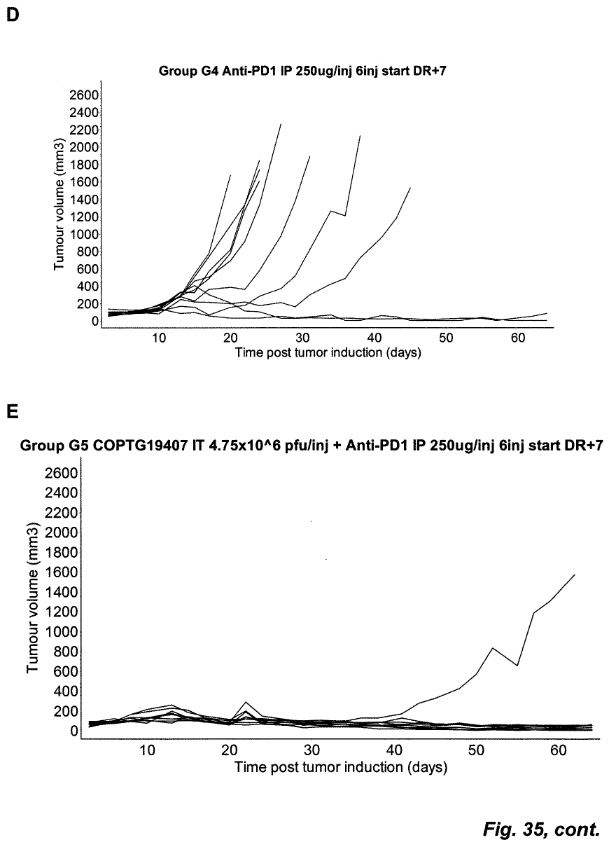

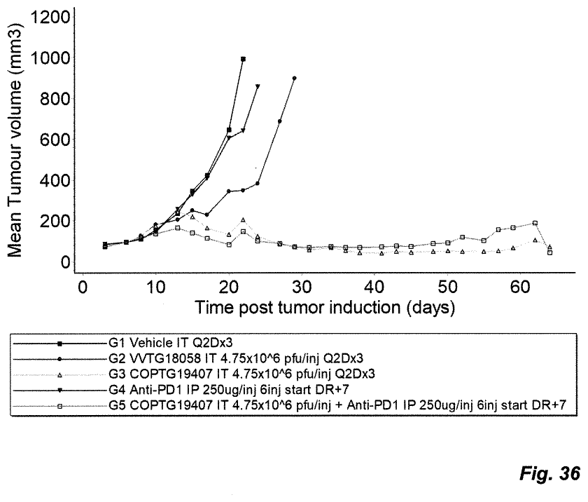

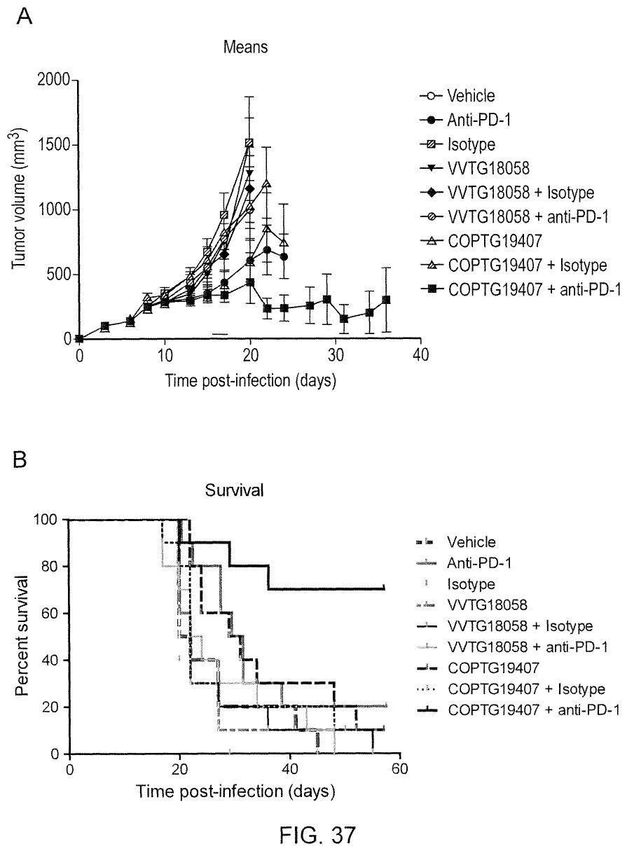

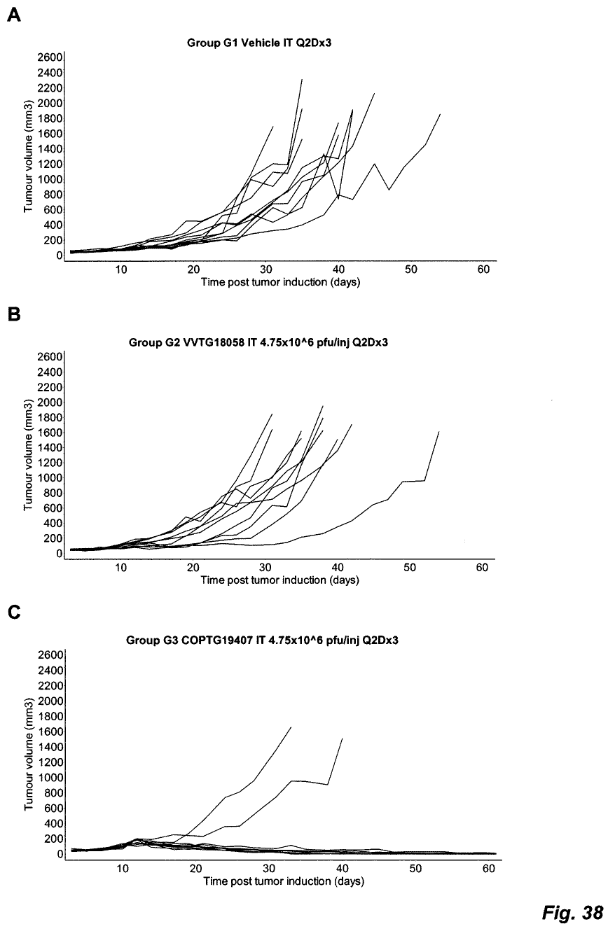

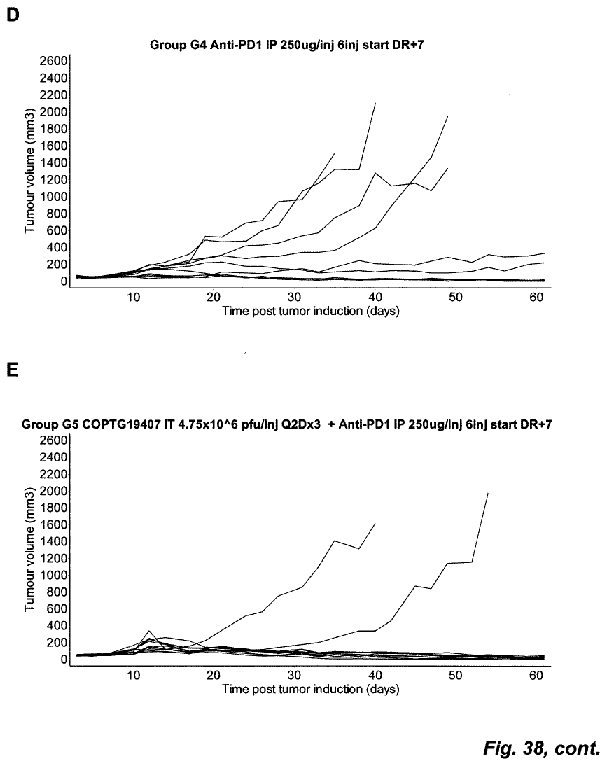

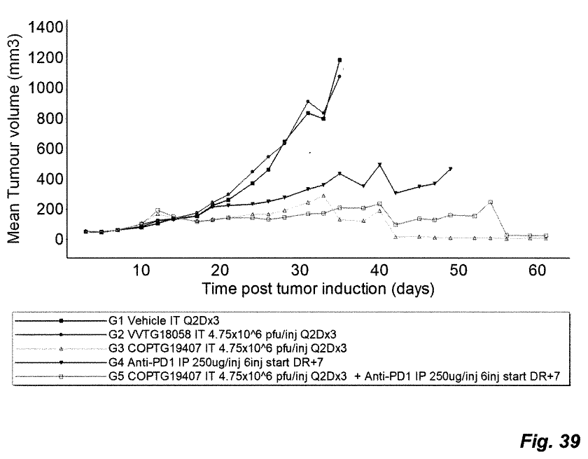

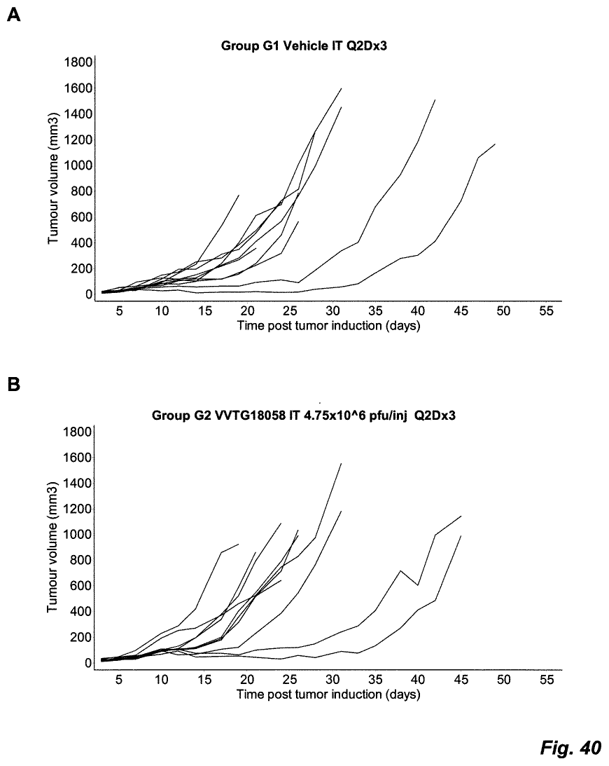

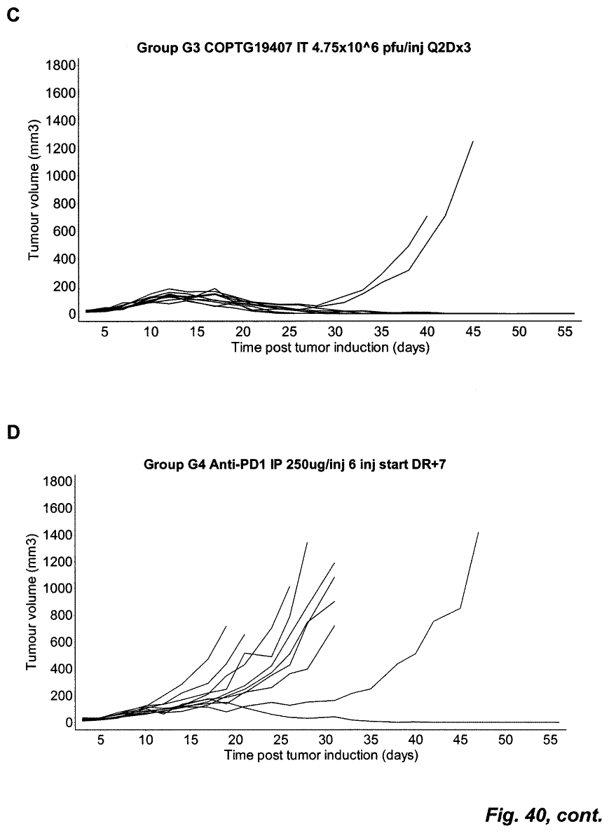

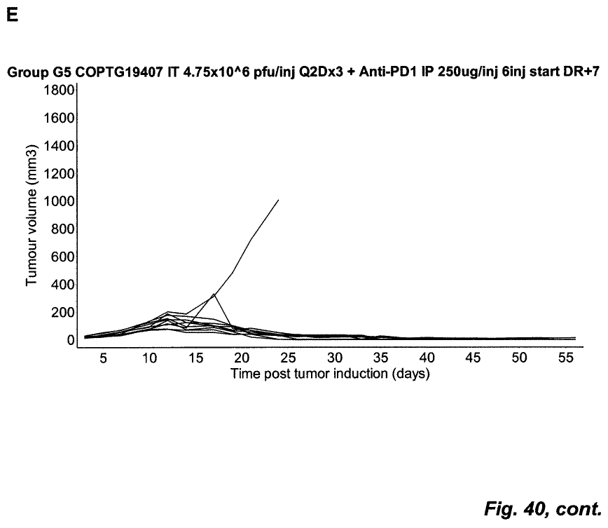

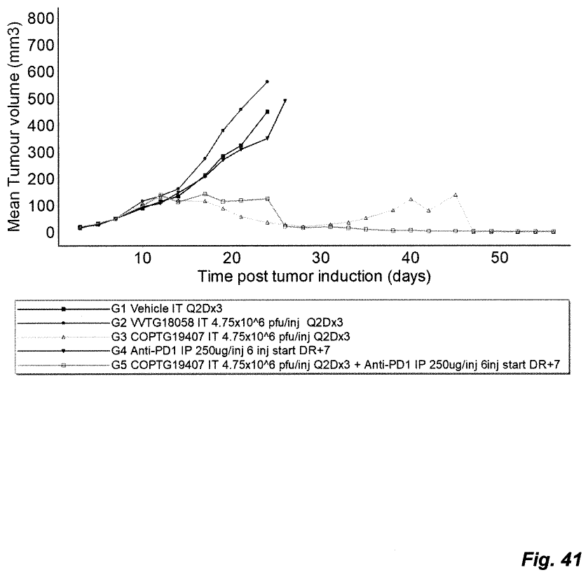

As shown in FIG. 13, CTLA-4 is highest expressed on intratumoral Treg cells which makes them a good target for depleting CTLA-4-specific antibodies.

### PBMC-NOG/SCID Model

To confirm the in vitro findings on the depleting activity of the CTLA-4 specific antibodies, we analysed the depleting capacity of anti-CTLA-4 mAb in a PBMC-NOG/SCID model in vivo. The model is based on the well-established hu-PBMC-NOG model (Søndergaard H. et al., Clin Exp Immunol. 2013 May; 172(2):300-10. doi: 10.1111/cei.12051; Cox J H et al., PLOS One. 2013 Dec. 23; 8(12):e82944. doi: 10.1371/journal.pone.0082944. eCollection 2013) and was modified in-house as described below.

Mice were bred and maintained in local facilities in accordance with home office guidelines. Eight weeks-old female C.B. 17 scid (Bosma G C et al., Nature. 1983 Feb. 10; 301(5900):527-30) and NOG (NOD/Shi-scid/IL-2Rγ^{null}; Ito M et al, 2002, NOD/SCID/γ_{c}^{null} mouse: an excellent recipient mouse model for engraftment of human cells. Blood 100(9):3175-3182) mice were supplied by Taconic (Bomholt, Denmark) and maintained in local animal facilities. For the PBMC-NOG/SCID (primary human xenograft) model, human PBMCs were isolated using Ficoll Paque PLUS and after washing the cells were resuspended in sterile PBS at 75×10⁶ cells/ml. NOG mice were i.v. injected with 200 μl cell suspension corresponding to 15×10⁶ cells/mouse. 2 weeks after injection, the spleens were isolated and rendered into a single cell suspension. Thereafter, a small sample was taken to determine the expression of CTLA-4 on human T cells by FACS. As indicated in FIG. 13, CTLA-4 is higher expressed on Treg cells compared to other T cells reflecting the situation in human patients. The majority of the cells was resuspended in sterile PBS at 50×10⁶ cells/ml. SCID mice were injected i.p. with 200 μl of the suspension corresponding to 10×10⁶ cells/mouse. 1h later, mice were treated with 10 mg/kg of either anti-CTLA-4 hIgG1, Yervoy or isotype control mAb. The intraperitoneal fluid of the mice was collected after 24 h. Human T cell subsets were identified and quantified by FACS using following markers: CD45, CD4, CD8, CD25, CD127 (all from BD Biosciences).

All antibodies tested showed a similar or better Treg depleting activity than Yervoy. Other T cell populations, such as CD8+ effector T cells, were not affected (FIG. 14).

### Example 5—The Selected Surrogate Antibody m5-B07 Shows the Same Functional Characteristics as 4-E03

In some of the examples, in particular the in vivo examples, the antibody clone 5-B07 in mIgG2a format has been used (also denoted m5-B07). This is a mouse antibody, which is a surrogate antibody to the human antibodies disclosed herein. It has been selected as a surrogate antibody since it binds murine CTLA-4 and thereby blocks ligand binding (FIG. 16 A-B). Furthermore, it also shows Treg depleting activity (FIG. 16 C-D).

### Ligand Blocking ELISA

The ligand blocking activity of 5-B07 was assessed by ELISA. To this end, recombinant mouse CTLA-4-Fc protein (Sino Biologocal Inc.) was coated to 96-well plates (Lumitrac 600 LIA plate, Greiner) at 1 pmol/well. After washing, titrated doses of anti-CTLA-4 mAbs were allowed to bind for 1 hour. His-tagged ligands were added at 200 nM and 100 nM, respectively (rmCD80 and rmCD86; Sino Biological Inc.) and the plates were further incubated for 15 minutes. After washing, bound ligand was detected with an HRP-labelled anti-His antibody (R&D Systems). Super Signal ELISA Pico (Thermo Scientific) was used as substrate and the plates were analysed using Tecan Ultra Microplate reader.

As shown in FIG. 16, the antibody blocks the binding of (A) CD80 and (B) CD86 to its ligand CTLA-4.

### Treg Depleting Activity In Vivo

The effects of CTLA-4 specific antibodies on the T cell subsets in the tumor in vivo was investigated in the CT26 tumor model as described below.

Mice were bred and maintained in local facilities in accordance with home office guidelines. Six to eight weeks-old female Balb/C were supplied by Taconic (Bomholt, Denmark) and maintained in local animal facilities. CT26 cells (ATCC) were grown in glutamax buffered RPMI, supplemented with 10% FCS. When cells were semi confluent they were detached with trypsin and resuspended in sterile PBS at 10×10⁶ cells/ml. Mice were s.c. injected with 100 μl cell suspension corresponding to 1×10⁶ cells/mouse. When the tumors reached approximately 7×7 mm, the mice were treated twice weekly i.p. with 10 mg/kg of the indicated antibodies as indicated in figures. After the third administration, tumors were dissected out, mechanichaly divided into small pieces and digested using a mixture of 100 g/ml liberase (Roche) and 100 μg/ml Dnase (Sigma) in 37° C. for 2×5 min with Vortex in between. The cell suspension was then washed (400 g for 10 min) with PBS containing 10% FBS. Thereafter, the cells were resuspended in MACS buffer and stained with an antibody panel staining CD45, CD3, CD8, CD4 and CD25 (all from BD Biosciences). Before staining, the cells were blocked for unspecific binding using 100 μg/ml IVIG. Cells were analyzed using a FACS Verse (BD Biosciences). Mouse Treg cells were identified as CD45⁺CD3⁺CD4⁺CD25⁺ cells.

As shown in FIG. 16 C, 5-B07 in mouse IgG2a format mediates Treg deletion in the tumor associated with D) increased CD8/Treg ratio compared to other CTLA-4 specific n-CoDeR antibodies and the well-described commercially available clone 9H10.

### Example 6-Generation of a Virus Expressing Anti-CTLA4 mAb (COPTG19385) or Anti-CTLA4 mAb and GM-CSF (COPTG19384), Expression of Transgenes and Characterization of Genetic Stabilities

COPTG19384 and COPTG19385 are vaccinia viruses (Copenhagen strain) encoding the monoclonal antibody anti-CTLA4 (4-E03). COPTG19384 further encodes the human GM-CSF. More particularly, COPTG19384 and COPTG19385 are both defective for thymidine kinase (TK, J2R locus) and ribonucleotide reductase (RR, I4L locus) activities. As illustrated in FIG. 17, the expression cassette encoding the 4-E03 heavy chain (HC; SEQ ID NO: 54) under the control of p7.5K promoter (SEQ ID NO: 59) was inserted at the J2R locus, and the expression cassette encoding the light chain (LC, SEQ ID NO: 53) of the 4-E03 IgG under the control of p7.5K promoter SEQ ID NO: 59) was placed at the I4L locus. For COPTG19384, the expression cassette encoding the human GM-CSF (SEQ ID NO: 56) under the control of pSE/L promoter (SEQ ID NO: 61) was also placed at the I4L locus.

The same promoter (p7.5K) was used to control the expression of HC and LC to obtain to same level of expression for both chains and therefore an optimal assembly of the antibody as a hetero-tetrameric protein (i.e. to avoid excess of non-associated chain). However, the same promoter for both chains of the antibody precludes from inserting them at the same locus (identical DNA sequences increase the risk of recombination and then elimination of transgenes). Therefore, the cassette encoding the 4-E03 HC was inserted at the J2R locus and the cassette encoding the 4-E03 LC at the I4L locus. The cassette encoding the GM-CSF transgene but under a different promoter (pSE/L) was also inserted into the I4L locus, like the antibody light chain.

### Generation of COPTG19384

The vaccinia virus transfer plasmids, pTG19339 and pTG19341, were designed to allow insertion of nucleotide sequences by homologous recombination in J2R and in I4L loci of the vaccinia virus genome, respectively. They originate from the plasmid pUC18 into which were cloned the flanking sequences (BRG and BRD) surrounding the J2R (pTG19339) or I4L (pTG19341) locus. Each plasmid contains also the p7.5K promoter.

A synthetic fragment named “Fragment HC” of 1436 bp containing the HC gene of 4-E03 antibody was produced. A fragment “LC fragment” containing the LC gene of 4-E03 antibody and hGM-CSF gene under the control of the pSE/L was generated by a synthetic way and inserted in a plasmidic vector. The coding sequences were optimized for human codon usage, a Kozak sequence (ACC) was added before the ATG start codon and a transcriptional terminator (TTTTTNT) was added after the stop codon. Moreover, some patterns were excluded: TTTTTNT, GGGGG, CCCCC which are deleterious for expression in poxvirus.

The HC fragment was inserted in pTG19339 restricted with PvuII by homologous recombination, giving rise to pTG19367. The LC-carrying plasmid was restricted by SnaB1 and the resulting fragment “LC-GMCSF” was inserted by homologous recombination in pTG19341 restricted with PvuII, giving rise to pTG19384. In this plasmid, the expressions cassettes were inserted head to tail between recombination arms allowing homologous recombination in the I4L locus of vaccinia virus genome.

COPTG19384 was generated on chicken embryo fibroblast (CEF) by two successive homologous recombination for successive insertion in I4L and J2R loci and by using COPTG19156 as parental virus and the two transfer plasmids pTG19367 and pTG19384. CEF were isolated from 12 day-old embryonated SPF eggs (Charles River). The embryos were mechanically dilacerated, solubilized in a Tryple Select solution (Invitrogen) and cultured in a MBE (Eagle Based Medium; Gibco) supplemented with 5% FCS (Gibco) and 2 mM L-glutamine.

The homologous recombination between the transfer plasmids and parental vaccinia virus enables the generation of recombinant vaccinia viruses which have lost the GFP and the mCherry expression cassettes and gained the antibody and GM-CSF expression cassettes. COPTG19156 contains the expression cassette of the mCherry gene in its I4L locus and the expression cassette of the GFP gene in its J2R locus. The homologous recombination between the transfer plasmid pTG19367 and the parental COPTG19156 enables the generation of recombinant vaccinia viruses which have lost their GFP expression cassette and gained the 4-E03 heavy chain expression cassette and the selection was performed by isolation of red fluorescent plaques. This intermediary recombinant virus (COPTG19367) was used as parental virus for a second round of homologous recombination with pTG19384 as transfer plasmid for the generation of recombinant vaccinia viruses which have lost their mCherry expression cassette and gained the 4-E03 light chain and GM-CSF expression cassettes. The selection of COPTG19384 (FIG. 17) was performed by isolation of white non-fluorescent plaques.

The viral stock of COPTG19384 was amplified on CEFs in two F175 flasks to generate appropriate stocks of viruses which can be aliquoted and stored at −80° C. until use. Viral stock was titrated on CEF cells and infectious titers were expressed in pfu/mL and calculated with the following formula: number of lytic areas x dilution factor×4. For illustrative purposes, the produced viral stock titrated 6.8×10⁶ pfu/mL. This stock was analyzed by PCR to verify the integrity of the expression cassettes and recombination arms using appropriate primer pairs. The stock was also analyzed by sequencing of both expression cassettes. Alignment of sequencing results showed 100% homology with the theoretical expected sequence. If needed, viral preparations were purified using conventional techniques (e.g. as described in WO2007/147528).

### Generation of COPTG19385

The vaccinia virus transfer plasmids, pTG19339 and pTG19341, were designed to allow insertion of nucleotide sequences by homologous recombination in J2R and in I4L loci of the vaccinia virus genome, respectively. They originate from the plasmid pUC18 into which were cloned the flanking sequences (BRG and BRD) surrounding the J2R (pTG19339) or I4L (pTG19341) locus. Each plasmid contains also the p7.5K promoter.

A synthetic fragment named “Fragment HC” of 1436 bp containing the HC gene of 4-E03 antibody was produced. The coding sequences were optimized for human codon usage, a Kozak sequence (ACC) was added before the ATG start codon and a transcriptional terminator (TTTTTNT) was added after the stop codon. Moreover, some patterns were excluded: TTTTTNT, GGGGG, CCCCC which are deleterious for expression in poxvirus.

The HC fragment was inserted in pTG19339 restricted with PvuII by homologous recombination, giving rise to pTG19367.

The plasmid containing the expression cassette encoding only the 4-E03 light chain was obtained by elimination of the cassette encoding the hGM-CSF gene under the control of the pSE/L in the plasmid pTG19384 (described above). pTG19384 was restricted with NheI and XbaI (compatible cohesive ends) and was religated, giving rise to pTG19385.

COPTG19385 was generated on chicken embryo fibroblast (CEF) by two successive homologous recombination for successive insertion in J2R and I4L loci and by using COPTG19156 as parental virus and the two transfer plasmids pTG19367 and pTG19385. CEF were isolated from 12 day-old embryonated SPF eggs (Charles River). The embryos were mechanically dilacerated, solubilized in a Tryple Select solution (Invitrogen) and cultured in an MBE (Eagle Based Medium; Gibco) supplemented with 5% FCS (Gibco) and 2 mM L-glutamine.

The homologous recombination between the transfer plasmids and parental vaccinia virus enables the generation of recombinant vaccinia viruses which have lost the GFP and the mCherry expression cassettes and gained the antibody expression cassettes. COPTG19156 contains the expression cassette of the mCherry gene in its I4L locus and the expression cassette of the GFP gene in its J2R locus. The homologous recombination between the transfer plasmid pTG19367 and the parental COPTG19156 enables the generation of recombinant vaccinia viruses which have lost their GFP expression cassette and gained the 4-E03 heavy chain expression cassette and the selection was performed by isolation of red fluorescent plaques. This intermediary recombinant virus (COPTG19367) was used as parental virus for a second round of homologous recombination with pTG19385 as transfer plasmid for the generation of recombinant vaccinia viruses which have lost their mCherry expression cassette and gained the 4-E03 light chain expression cassette. The selection of COPTG19385 was performed by isolation of white non-fluorescent plaques.

The viral stock of COPTG19385 was amplified on CEFs in two F175 flasks to generate appropriate stocks of viruses which can be aliquoted and stored at −80° C. until use. Viral stock was titrated on CEF cells and infectious titers were expressed in pfu/mL and calculated with the following formula: number of lytic areas×dilution factor×4. For illustrative purposes, the produced viral stock titrated 1.04×10⁷ pfu/mL. This stock was analyzed by PCR to verify the integrity of the expression cassettes and recombination arms using appropriate primer pairs. The stock was also analyzed by sequencing of both expression cassettes. Alignment of sequencing results showed 100% homology with the theoretical expected sequence. If needed, viral preparations were purified using conventional techniques (e.g. as described in WO2007/147528).

### Expression of Transgenes

Virus-mediated expression of 4-E03 monoclonal antibody was evaluated in supernatants of CEF cells infected with COPTG19384 by Western Blot (WB) and compared to the recombinantly produced antibody (40 ng of 4-E03). WB allows to visualize the presence of non-functional molecules that do not bind the CTLA4 (e.g. molecules with incomplete chain assembly, aggregates). CEF cells were infected at MOI 0.05 with COPTG19384 viral stock in triplicate. Cell supernatants were harvested after 48 h and were analyzed by WB after an electrophoresis in non-reducing condition and using either an anti-Ig (left blot) or an anti-light chain (right blot) HRP conjugated antibody. The results illustrated in FIG. 18A indicate that the WB profile in non-reducing condition of the mAb produced by infected CEF is close to that of the purified 4-E03 with a similar apparent size between 100 to 150 kDa, indicating a correct chains-folding and assembly.

Quantification in supernatants of the functional secreted 4-E03 antibodies and GM-CSF was performed with ELISA. ELISA allowed to measure quantitively the amount of functional polypeptide produced in cell supernatants. VVTG17137 was used as a negative control. It is a vaccinia virus (Copenhagen strain) deleted in J2R and I4L loci encoding the suicide gene FCU1 (described in WO2009/065546).

For estimation of 4-E03 antibody, microplates were coated by an overnight incubation at 4° C. with 100 μL per well of CTLA4-Fc at 0.25 μg/mL. After incubation, the coating solution was discarded, blocking solution was added and plates were incubated for 1 to 2 hours at RT before being washed. 4-E03 calibration standards (from 0.097 to 100 ng/ml), or samples (in triplicate) diluted in blocking solution were added to the wells and the plates were incubated for 2 hours at 37° C. before being washed. HRP conjugated antibody diluted in blocking solution was added to each well and plates were incubated 1 hour at 37° C. before being washed. After incubation with TMB solution 30 min at RT in darkness, H₂SO₄ 2 M (stop solution) was added to stop the enzymatic reaction. Absorbances were read at 450 nm on microplate reader. Absorbances were plotted versus antibody concentration of the calibration standard. As illustrated in FIG. 18B, functional 4-E03 mAb was produced in cells infected by COPTG19384, reaching concentrations close to 1 μg/mL.

Virus-mediated expression of GM-CSF was also evaluated in the same supernatants using Quantikine® ELISA (R&D Systems Ref SGM00). Briefly, this assay is using two anti-hGM-CSF antibodies. The first antibody used to capture the hGM-CSF in the samples was coated on the well surface of a 96-well plate. The second one is conjugated and added to the plate in solution to detect the captured hGM-CSF. The concentration of hGM-CSF in the sample is then calculated by interpolation from a calibration curve established with some purified hGM-CSF provided by the kit. As illustrated in FIG. 19, the level of expression of GM-CSF was around 6 μg/mL.

In conclusion, concentrations equal or above 1 μg/mL were detected for both transgenes indicating a satisfying level of expression.

### Genetic Stability:

Genetic stability tests were performed after five passages of the virus on CEF in serum free medium at a multiplicity of infection (MOI) of 10-4. Passage P5 was diluted and inoculated on CEF cells in 60 mm culture dishes to obtain from 20 to 40 viral plaques per dish. One hundred viral plaques were isolated and sub-cultured. After one amplification cycle, isolated viral plaques were inoculated on CEF cells and tested by PCR and by ELISA. PCR analysis and expression of transgenes showed that more than 90% of clones have a correct profile. As the the acceptance criteria for clinical development of a product is a genetic stability superior or equal to 90%, COPTG19384 was considered as genetically stable.

### Example 7—In Vitro Characterization of COPTG19384 Replication Studies in Hepatocytes: Tumor-Selectivity of a Virus Expressing Anti-CTLA4 mAb and GM-CSF

COPTG19384 carries two gene deletions encoding viral enzymes (TK and RR) involved in nucleotide metabolism. When functional, these enzymes allow the virus to replicate in cytoplasm of most of cells including the ones in resting state (i.e. with low nucleotide pool available). A host range study was performed in primary and malignant cells to verify that the insertion of the different transgenes in the J2R and I4L loci does not modify the host range selectivity. The replication of COPTG19384 was assessed on normal primary human cells (hepatocytes, prepared by Biopredic) and on tumoral cells from the same organ (HepG2 from hepatocarcinoma, ATCC® HB-8065™). Replication rates and therapeutic indexes were calculated and compared to those of both the wild type Copenhagen vaccinia (COP WT, virus without any deletion) as references for non-selective vaccinia virus and the recombinant double deleted VVTG17137 virus (deleted in J2R and I4L genes with the suicide FCU1 gene inserted in place of J2R), respectively. Two batches were assayed, a research batch (batch1) and a GMP-produced batch (batch 2). The replication rate was determined as the ratio of total infectious particles at the end of incubation/initial infectious particle (inoculum). The therapeutic index of each virus was determined as the ratio: replication rate on HepG2 cells/replication rate on hepatocytes. The higher the ratio the better the selectivity of the virus toward the tumoral cells.

Primary hepatocytes were grown in Basal Hepatic cell medium supplemented with 1.6% Additives for hepatocyte culture medium. HepG2 were seeded in 12 well plate at 4 E+05 cells/well and incubated for 24 H at 37° C. with 5% CO₂. Before infection the culture medium was removed and 70 pfu/well of virus in either PBS for hepatocytes or FCS-supplemented PBS for HepG2 were added to each well. The infected cells were incubated 30 min at 37° C. with 5% CO₂ and then 1.5 mL/well of culture medium were added. Plates were incubated at 37° C. with 5% CO₂ for 3 days and then stored at −80° C. Then, plates were thawed and wells sonicated 30 seconds with 40% of amplitude before titration on Vero cells.

### Replication Rate in Normal Human Hepatocytes:

Normal hepatocytes were chosen to monitor the capacity of COPTG19384 in normal human cells as these primary cells can be obtained regularly directly from donors. In those cells, the COP WT spread well with a replication rate of more than 50,000 (FIG. 20A). In other words, each initial infection viral particle produced about 50,000 new viruses. In the case of the two recombinant double deleted viruses (i.e. VVTG17137 and COPTG19384), this replication rate was dramatically reduced to 5 to 15 according to the virus or batch of virus (FIG. 20A). This last result indicates that the attenuated replication toward normal cells brings along by the two deletions was conserved between VVTG17137 and COPTG19384.

### Replication Rate in Tumoral Cells HepG2:

HepG2 cells were chosen to monitor the capacity of COPTG19384 to replicate in tumoral human cells as these cells are a malignant counterpart of normal hepatocytes. In those cells, the five viruses tested had a quite similar replication rate reaching about 100,000 new viruses whatever the initial virus tested (FIG. 20B). Therefore, the double deletion in both VVTG17137 and COPTG19384 and vectorization of transgenes did not impair their capacity to replicate in malignant cells.

### Therapeutic Index:

As illustrated in FIG. 20C, the calculated index is only two for COP WT indicating a poor selectivity of the COP WT for the tumoral versus normal cells. In contrary, for both VVTG17137 and COPTG19384 (and for both lots of virus tested) this index varies from 8.2 E+03 to 1.8 E+04. This confirms that the two recombinant viruses have the same good selectivity toward tumoral versus normal cells.

These results demonstrated that COPTG19384 and VVTG17137 have a very similar replicative properties on both tumoral and healthy cells. Compared to COP WT, their replication on tumoral Hep G2 is similar whereas it is highly impaired on healthy hepatocytes. Therefore, the deletion of the two genes (J2R and I4L) restricts the replication of the deleted virus to the multiplying cells (i.e. with high nucleotide pool) including the tumoral cells. Since the transgenes expression and the replication are tightly linked, COPTG19384 is an efficient vector for the selective delivery of therapeutic proteins into tumour.

### Replication Assays on CEF and LoVo:

Replication of COPTG19384 was evaluated on CEF (producer cells) isolated from 11 or 12 day-old embryonated Specific Pathogens Free eggs (Charles Rivers) and on a tumoral human cell line (LoVo; ATCC® CCL-229™). CEF and LoVo cells were prepared in suspension and infected at MOI of 10⁻³ for CEF and 10⁻² for LoVo (three wells per cells and per time point). After different times of incubation, Viral titration was done on Vero cells (CCL-81™). COPTG19384 replication was compared to the one of VVTG17137 as benchmark. The results show that the replication of COPTG19384 and VVTG17137 were similar in both CEF and LoVo (data not shown).

### Replication Assays on Reconstructed Human Skin:

Replication of COPTG19384 was also evaluated on reconstructed human skin (T-Skin™/Human Full Thickness Skin Model). Thirty-six T-Skin™ samples obtained from (EPISKIN SA) were cultured in 6-well plates and maintained in fresh culture medium. VVTG17137 and COPTG19384 were distributed into each well (in triplicate) in order to obtain the attended final concentration (i.e. 10¹ to 10⁵ pfu/well). A negative control corresponding of medium without virus was also tested (Mock). Plates were incubated at 37° C. with 5% CO₂ for 7 days and T-Skin™ samples were collected and cut in two pieces. Infectious titer was determined on one of the two pieces using Vero cells for virus titration. FIG. 21 shows that COPTG19384 replicates in reconstructed skin to the same extent as the benchmark VVTG17137 supporting the fact that the vectorization of both GM-CSF and 4-E03 mAb did not modify the replication behavior of the vaccinia virus on the human reconstructed skin.

### Oncolytic Assay

Oncolytic activity is representative of the lytic activity of the tested viral samples on tumor cells. It was assessed by quantification of cell viability after 5 days of incubation on different tumor cell lines: the human colorectal adenocarcinoma cell line LoVo (ATCC® CCL-229™), the human pancreatic tumor cell line MIA PaCa-2 (ATCC® CCL-1420) and the human hepatocarcinoma cell line HepG2 (ATCC® HB-8065™). COPTG19384 oncolytic activities were compared to the ones of VVTG17137 as benchmark. A negative control corresponding of uninfected cells was also plated (Mock infected cells).

Cells were prepared, distributed in Eppendorf tubes (1.2×10⁶ cells/tube) before being infected with the virus at a MOI of 10⁻⁵ to 10⁻² and incubated 30 min at 37° C. Appropriate complete medium was added to Eppendorf tube and an aliquote of this suspension was added in each well (in triplicate) in 6-well plate containing 2 mL of appropriate complete medium. Plates were incubated at 37° C. with 5% CO₂ for 5 days and cell viability was determined on Vi-Cell counter. Results were expressed as a percentage of the cell viability of mock infected cells. The cell supernatants were also recovered for the determination of concentration of 4-E03 mAb and GM-CSF. FIG. 22 shows that the oncolytic activities of COPTG19384 and VVTG17137 are similar in the three tumoral cell lines assessed.

### Level of Expression of Transgenes

The levels of expression of both 4-E03 monoclonal antibody and GM-CSF were measured by ELISA (as described in Example 6) in culture supernatants of HepG2 and LoVo cells recovered after oncolysis activity determination (5 day of infection at variable MOI).

The levels of expression of 4-E03 and GM-CSF were also measured by ELISA (see example 6) in supernatants of 5 cell lines cultured in the following conditions, respectively the human gastric carcinoma cell line Hs-746 T (ATCC® HTB-135™), the human ovarian tumor cell line SK-OV-3 (ATCC® HTB-77™), the human pancreatic tumor cell line MIA PaCa-2 (ATCC® CCL-1420), the human colorectal adenocarcinoma cell line LoVo (ATCC® CCL-229™) and the human colorectal carcinoma cell line HCT 116 (ATCC® CCL-247™). Each cell line was cultured (in triplicate) in 6-well plates (10⁶ cells/well) and incubated at 37° C. with 5% CO₂ for 24 h before being infected at MOI 0.05. The cell supernatants were then recovered 48 h post infection for the determination of concentration of 4-E03 mAb and GM-CSF. As expected, the MOI, time post-infection and cell line are important parameters that impact the level of transgenes expression in supernatants of infected cells. FIG. 23A shows that a rather permissive to replication (HepG2) and a rather resistant (LoVo) tumoral cell line, when infected by COPTG19384, are able to produce in their culture supernatants approximately the same amount of 4-E03 mAb and GM-CSF. However, the maximum of expression for the HepG2 is reached at a MOI 10-fold lower than for the LoVo. Moreover, for the 5 tumoral cell lines tested, the expression of transgenes was above 0.1 and above 1 μg/mL for 4-E03 mAb and GM-CSF respectively (FIG. 23B). To be noted that the ELISA assay used to measure the concentration of 4-E03 mAb uses the antigen CTLA4 to capture the antibody. In other words, the antibodies measured by this assay are at least partially functional (i.e. recognizing their antigen, the other functions of the antibody are carried by the Fc part),

### Purification of 4-E03 mAb and Glycosylation Profile Analysis

In order to produce a rather large quantity of 4-E03 mAb from infected cells, 15 F175 flasks containing ˜4.7 107 Mia-PACA cells/flask were infected at MOI 0.01 with COPTG19384 and incubated 72 h. MIA Paca-2 cell culture supernatant (about 450 mL containing 670 μg of mAb 4-E03 as determined by ELISA) was harvested, pooled and clarified by centrifugation to remove most of the cell debris. Cleared supernatants were filtered on 0.2 μm filters and 2 mM EDTA (to inhibit putative metal proteases) and 20 mM Tris pH7.5 (to raise the pH) were added. The filtered supernatant was then passed through a protA Hitrap column (GE healthcare, ref 17-5079-01). The column was transferred and connected to Purifier FPLC (GE Healthcare) and purification program (THM/ProtA 1 mL injection loop frac bleu) was applied. The eluted fractions containing the mAb were loaded on NuPage Bis-Tris gels 4-12% (Thermo NP0323) after addition of Laemlli buffer (Biorad) containing or not beta-mercaptoethanol for reduction or not of the disulphide bonds of the mAb. The gel was stained with InstantBlue (Expedeon, ISB1L). Three fractions corresponding to the main peak of elution were pooled and, after dialysis against the formulation buffer, the antibody concentration was determined by absorbance at 280 nm. The final concentration of the purified mAb was 0.29 mg/mL.

The first characterization was an assessment of chains assembly by electrophoresis in reducing and non-reducing conditions. In non-reducing conditions the 2 light and 2 heavy chains assemble to form the native and functional antibody. It appeared that the purified 4-E03 from infected MIA PaCa-2 and the recombinantly produced 4-E03 have an undistinguishable electrophoresis profiles in both reducing and non-reducing conditions. In other words, the purified 4-E03 from infected MIA PaCa-2 has the expected ratio of light and heavy chains and is correctly assembled into 2 light and 2 heavy chains heterotetramer. The presence of this heterotetramer was also confirmed by mass spectrometry. The purified mAb was subject to mass spectrometry for glycosylation analysis. Briefly, the mAb was digested, or not, with IdeS protease that cleave specifically the IgG at the hinge (resulting in F(ab′)₂ and Fc parts). The masses of the whole antibody or Fc parts that carry the N-glycosylation were determined and each mass was fitted with a theoretical mass calculated from the primary sequence of Fc and a glycosylation pattern. The glycosylation profile of 4-E03 mAb purified from infected MIA PaCa-2 was compared to the one of the recombinantly produced and purified 4-E03 and Mab Thera as benchmark for human IgG1 used in clinic. The results show that the glycosylation profiles of 4-E03 produced from infected MIA PaCa2 had a glycosylation profile different from the two antibody references with a majority of G0F (88%) whereas both recombinant 4-E03 and MabThera have a similar glycosylation profile with the typical G0F, G1F and G2F distribution. However, MIA PaCa-2 was suspected to be the cause of the low G1F and G2F species in the purified 4-E03 mAb due to a low level of Beta-1,4-Galactosyltransferase 1 transcript which could be the cause of the lack of galactosyl moiety (and therefore a lack of Gif and G2F) of the 4-E03 expressed in MIA PaCa-2.

The same type of purification followed by mass analysis was also performed from permeat recovered during the purification of the COPTG19384 produced on CEF. The results show that the glycosylation profiles of 4-E03 from infected CEF was very similar to the ones of MabThera or recombinant 4-E03. This latest result suggests that the glycosylation profile of the antibody is more impacted by the cell line used than by the infection itself.

4-E03 purified from the supernatant od infected MIA PaCa-2 cells (4-E03 TG) also exhibit the same binding characteristics as recombinantly produced 4-E03 by CHO (research batch) or HEK (tox batch) cells (FIGS. 24 and 25). This was demonstrated by ELISA (described in Example 1) to test the binding to recombinant (FIG. 24A) human and (FIG. 24B) cynomolgus CTLA-4 protein. A FACS analysis where the binding to (FIG. 25A) human and (FIG. 25B) cynomolgus CTLA-4 expressing cells was tested (see Example 1 and 2) confirmed similar cross-reactivity and binding affinities for the different 4-E03 batches.

### GM-CSF Glycosylation and Disulphide Bonds Pattern

In order to investigate the glycosylation pattern and the presence of some disulphide bonds, different human tumoral cells line were infected by COPTG19384 and their supernatants were analysed by the same WB method. MIA-Paca-2, LoVo, HepG2 and HCT116 cells were infected at MOI 0.01 and incubated 72 h in culture medium without serum. The culture supernatants were harvested, clarified by centrifugation and then filtered on 0.2 μm filter. The supernatants were stored at −20° C. until analysis. They were treated by addition 8 μl of Rapid PNGase F Buffer 5× followed by an incubation at 75° C. for 5 minutes. One μL of PNGase F was then added (to remove N-glycans from glycoprotein) and the mixture incubated 30 minutes at 50° C. Twenty-five μL of samples were prepared by addition of 5 μL Laemmli buffer×4 with or without beta-mercaptoethanol (reducing and non-reducing conditions) before being submitted to western blotting. Immune complexes were detected using the Amersham ECL Prime Western Blotting and chemiluminescence was recorded with a Molecular Imager ChemiDOC XRS (Biorad).

GM-CSF from infected HCT116, LoVo and MIA PaCa-2 displayed the same pattern of glycosylation whereas the GM-CSF produced by infected HepG2 migrated as a non-N-glycosylated molecule. These results indicate that the GM-CSF produced by the COPTG19384 infected human tumoral cells has the expected post-translational modifications (i.e. disulphide bonds and N-glycosylation). However, these modifications probably vary depending of the tumour cell lines used for infection and their specific metabolic status.

### Example 8: Pharmakokinetics after Intratumoral Injections of COPTG19384

Kinetic of Expression in Tumor and Bloodstream of Anti-CTLA4 Antibodies, GM-CSF and Viruses after Intratumoral (i.t.) Injection of Vaccinia Viruses in a LoVo Xenografted Model.

### Protocol

5×10⁶ cells LoVo cells were implanted in right flank of Swiss nude mice (Charles River, France). After about two weeks when the tumours volume reached ˜120 mm³, the mice were randomized and split in 6 groups of 15 animals.
Mice from group 1 received an i.t. administration of COPTG19384 at a dose of 1×10⁴ pfu/mouse at D0 (first day of treatment).
Mice from group 2 received an i.t. administration of COPTG19384 at a dose of 1×10⁵ pfu/mouse at D0.
Mice from group 3 received an i.t. administration of VVTG17137 at a dose of 1×10⁴ pfu/mouse at D0.
Mice from group 4 received an i.t. administration of VVTG17137 at a dose of 1×10⁵ pfu/mouse at D0.
Mice from group 5 received an intraperitoneal (i.p.) administration of 4-E03 at a dose of 3 mg/kg at D0.
Mice from group 6 received an i.p. administration of Ipilimumab (Yervoy) at a dose of 3 mg/kg at D0.

Tumour and blood from 3 animals were collected at days 1, 3, 6, 10 and 20. Tumors were weighted and homogenized for immediate processing. One quarter of the homogenized tumours was collected for virus titration and the remaining suspension was centrifuged and supernatants were stored at −20° C. until use. Blood was split in two parts: one was added to heparin tube (25 IU/100 μL of blood) for titration assay and frozen at −80° C. until analysis. Clarified sera were produced from the other part and stored at −20° C. until use. Virus titer was determined in tumor and blood samples by titration on Vero cells.

### Kinetic of Virus Replication in LoVo Model:

In the LoVo model, where COPTG19384 was injected once at two doses (1×10⁴ or 1×10⁵ pfu) the virus replication was monitored and compared to the one of VVTG17137 injected in same conditions. The results displayed in FIG. 26 show an important dispersion of the three values of virus titers measured for each time point. Anyway, the results show also that both viruses and at both doses replicate in the tumour and maintain a rather high titer/g of tumour from day 3 to up to 20 days after the injection. There is no obvious difference of virus titer, for a given time point, between the two doses of virus or between the two viruses used. Interestingly, all the blood samples were negative for virus detection excepted one sample (VVTG17137, dose: 1×10⁷ pfu at Day 10) for which only 13 pfu/mL were detected (data not shown).

Together these results indicate that, after one i.t. injection of either 1×10⁴ or 1×10⁵ pfu, the virus' replication was maintained in LoVo tumours for at least 20 days with a barely detectable presence in the blood stream. It has to be noted that the LoVo xenografted model is very favourable to the virus replication as it uses permissive human tumour cells and Swiss nude mice that have a severely impaired immune system with, therefore, a limited anti-viral activity.

### Kinetic of Transgenes Expression in LoVo Model:

As expected, the kinetic of transgenes expression in tumour followed kinetic of virus replication with a maximum concentration (Cmax) at days 6 or 10 for both 4-E03 mAb (FIG. 27A) and GM-CSF (FIG. 27B). In the case of the single injection of 4-E03 mAb (or Ipilimumab), the Cmax in tumour and blood were observed at the first time point (day 1) and the thereafter measured concentrations of mAb were in accordance with the pharmacokinetic of a human IgG1 in mouse (FIG. 28).

Moreover, the concentrations of 4-E03 into the tumour at Cmax and thereafter (i.e. from 6-10 to 20 days after injection) was around 10-fold higher following COPTG19384 treatment (for both doses) than after a single injection of 4-E03 mAb at a therapeutic dose of 3 mg/kg (FIG. 27A). In contrast, the concentration in blood of the mAb after COPTG19384 treatment was always inferior to those measured after i.p. injection of 3 mg/kg of 4-E03 (FIG. 28A). This result indicates that vectorization of mAb allows to reach high concentration into the tumour without exceeding or even reaching the blood concentration obtained at therapeutic dosing of mAb.

The kinetic of expression of GM-CSF after COPTG19384 treatment follows the one observed with 4-E03 (FIG. 27B). Interestingly, the levels of GM-CSF measured into the tumour are below the level of 4-E03 for the same samples, although in vitro LoVo infected by COPTG19384 express about two-fold more GM-CSF than 4-E03. The blood concentrations of GM-CSF were also very low compared to the 4-E03 ones (FIG. 28B). This result is in accordance with the in vivo half-life of GM-CSF that is very short compared to the one of a human IgG1.

These results indicate that the vectorized antibodies and GM-CSF are expressed mainly in the tumour after i.t. injection of COPTG19384 with a minimal systemic exposure. These results confirm that vectorization is particularly suitable for transgenes with toxicological (e.g. anti-CTLA4) or pharmacokinetic (e.g. GM-CSF) issues.

Kinetic of Expression in Tumor and Bloodstream of Anti-CTLA4 Antibodies, GM-CSF and Viruses after Intratumoral Injection of Vaccinia Viruses in a CT26 Syngeneic Model

Evaluation of the viral activities in the CT26 immunocompetent murine model requires the generation of several surrogate viruses encoding a murine anti-mCTLA4 with or without the murine GM-CSF:
COPTG19407 is a Vaccinia virus (Copenhagen strain) containing an expression cassette encoding the heavy chain of the murine m5-B07 IgG2 (SEQ ID NO: 63) under p7.5 promoter at the J2R locus, and an expression cassette encoding the light chain of the m5-B07 (SEQ ID NO: 62) under p7.5 promoter and murine GM-CSF (SEQ ID NO: 58) under pSE/L promoter at the I4L locus.
COPTG19421 is a Vaccinia virus (Copenhagen strain) containing an expression cassette encoding the m5-B07 heavy chain under p7.5 promoter at the J2R locus, and an expression cassette encoding the light chain of the m5-B07 under p7.5 promoter at the I4L locus.
VVTG18058, used as benchmark, is a Vaccinia virus (Copenhagen strain) deleted in J2R and I4L genes, without any transgene (“empty” virus).

These vaccinia viruses were generated as for the human counterparts by two successive homologous recombinations at J2R (TK) and then I4L (RR) loci following the process described in example 6. The ELISA method to quantify the m5-B07 antibody and mGM-CSF was similar to that described above (example 6, “expression of transgene” for 4-E03 and GM-CSF) except that murine CTLA4-Fc antigen was used to capture the murine antibody and Quantikine ELISA kit Mouse GM-CSF (R&D Systems) was used to quantify mGM-CSF. Oncolytic activity of these virus was also evaluated in various cell lines (one sarcoma: MCA205 and two colon carcinoma CT26 and MC38) and found similar to the one of VVTG18058 showing that vectorization of the murine antibody with or without the mGM-CSF did not impact the oncolytic abilities of the vaccinia virus (data not shown).

Protocol: CT26 cells (2×10⁵ cells) were implanted in right flank of Balb/c mice (Charles River, France). After about one week when the tumours volume reached 25-50 mm³ the mice were randomized and split in 3 groups of 20 animals (groups 1 to 3) and one group 4 of 10 animals. Tumor and blood were collected and treated as described for the LoVo model except that they were collected at days 1, 4, 8 and 10 for the first 3 groups and day 1 for the group 4.
Mice from group 1 received an i.t. administration of VVTG18058 at a dose of 1×10⁷ pfu/mouse at D0, D2 and D4.
Mice from group 2 received an i.t. administration of COPTG19407 at a dose of 1×10⁷ pfu/mouse at D0, D2 and D4.
Mice from group 3 received an i.t. administration of COPTG19421 at a dose of 1×10⁷ pfu/mouse at D0, D2 and D4.
Mice from group 4 received an i.p. administration of m5-B07 at a dose of 3 mg/kg at D0.

### Kinetic of Virus Replication in CT26 Model:

In the CT26 model, where two surrogate viruses were injected thrice (1×10⁷ pfu/injection) the virus replication was monitored and compared to the one of VVTG18058 injected in the same conditions. The results displayed in FIG. 29 show, as for the LoVo model, an important dispersion of the three values of virus titers measured for each time point. However, the titers for the three viruses were maintained over the time and up to 10 days, indicating that the two transgenes did not impact the virus clearance or replication, at least in this window of time. No virus infectious particle was detected in any of the blood samples (data not shown).

### Kinetic of Transgenes Expression in CT26 Model

Like for the LoVo model, the transgenes expression into the tumour mirrored the virus replication. In other words, the m5-B07 antibody (FIG. 30A) and mGM-CSF (FIG. 30B) were detected in the tumour at a rather constant level over the 10 days of the monitoring.

In the case of the monoclonal antibody, the Cmax reached after either COPTG19421 or COPTG19407 injections were about 10-fold lower than the Cmax observed with a single i.p. injection of the m5-B07 antibody at 3 mg/ml (FIG. 30A). In the serum, the difference was even more pronounced with a circulating concentration of m5-B07˜100-fold lower after virus treatments versus m5-B07 injection at 3 mg/kg (FIG. 31).

For GM-CSF, only the treatment by COPTG19407 yielded to measurable concentration of mGM-CSF in CT26 tumors indicating that the measured cytokine had a recombinant, rather than endogen, origin. Like in LoVo model, the mGM-CSF concentrations measured in tumour were lower than the ones of m5-B07 (FIG. 30B). Moreover, the mGM-CSF produced by the tumour was not detectable in any of sera sample probably due to a short half-life of the molecule that preclude any systemic accumulation.

### Example 9: Antitumoral Activity Studies

COPTG19347 is a Vaccinia virus (Copenhagen strain) deleted in J2R and I4L genes and encoding a whole murine antibody (namely m5-B07, heavy and light chain) recognizing the murine CTLA4 antigen. COPTG19421 versus COPTG19347 expressed both m5-B07 but under different promoters: namely p7.5K and pH5.R respectively. Quantification of m5-B07 was assessed in supernatants of infected cells reaching about 1 μg/mL in infected CT26 at MOI 10-1 to about 4 μg/mL in infected MCA205 cells at MOI 102. The higher expression in MCA205 versus CT26 was observed also for mGM-CSF in the culture supernatant of cells infected by COPTG19407 (data not shown).

Antitumoral Activity in Mice Bearing CT26 Model in Combination with Anti-PD1 Protocol:

CT26 cells (2×10⁵ cells) were implanted in right flank of Balb/c mice (Charles River, France). When tumors reached a volume of 25-50 mm³, the mice were randomized in five groups of ten animals. Briefly, the mice were treated by three i.t. administrations, 2 days apart, of virus followed of i.p. treatment of murine anti-PD1 (RMP1-14 BioXcell) twice a week for three weeks. More specifically,
Mice from group 1 received vehicle;
Mice from group 2 received an i.t. administration of 1×10⁷ pfu of COPTG19347 at D0, D2 and D4;
Mice from group 3 received an i.t. administration of 1×10⁷ pfu of COPTG19347 at D0, D2 and D4 and i.p. intraperitoneal administration of 250 μg/mice of RMP1-14, at D7, D11, D14, D18 and D22;
Mice from group 4 received an i.p. administration of 250 μg/mice of RMP1-14, at D7, D11, D14, D18 and D22;
Mice from group 5 received an i.t. administration of 1×10⁷ pfu of VVTG18058 at D0, D2 and D4.

Tumor dimensions were measured twice a week with calipers and their volumes calculated using the formula (n/6) (length×width²). The animals were euthanized when their tumor volume reached 2000 mm³.

### Antitumoral Activities of COPTG19347 in CT26 Model:

As illustrated in FIG. 32, COPTG19347 treatment yielded not only to tumour growth inhibition (FIG. 32A) but also to tumour regression that finally translate into tumour free mice that survive up to 100 days (FIG. 32B). Treatment with COPTG19347 yielded to 60% tumour free mice at day 100. The co-treatment with anti-PD-1 antibody did not improve significantly the tumour growth inhibition or the percentage of long surviving mice (about 70% tumour free mice at day 100). In comparison, RPMI-14 treatment did not provide any anti-tumor effect (same behavior as non-treated mice (all dead within the first 40 days) whereas VVTG18058 had a poor activity (about 10% tumour free mice at day 100).

### Dose-Effect Evaluation in CT26 Model:

The three surrogate viruses were compared (different promoter to drive m5-B07 and with or without m-GM-CSF) and a dose escalation of COPTG19407 versus VVTG18058 was performed (7.5×10⁴, 7.5×10⁵ or 7.5×10⁶ pfu). The experimental conditions were exactly as the ones described above except that the co-treatment with anti-PD1 was omitted.

The results of two independent experiments demonstrated clearly that the three viruses tested COPTG19407, COPTG19421 and COPTG19347 had a strong anti-tumoral activity at the dose of 7.5×10⁶ pfu. This confirm that neither the mGM-CSF encoded in COPTG19407 nor the use of a weakest promoter in COPTG19421 and COPTG19407 did impair the anti-tumoral activity of the armed viruses. At the highest dose tested (i.e. 7.5×10⁶ pfu), the number of tumor free mice at 80 days were between 5/10 and 7/10 depending of the virus and of experiment versus 0/10 for the mice treated with the empty virus as summarized in the following Table.

TABLE 6
Effect of the surrogate viruses COPTG19407, COPTG19421 and
COPTG19347 on the tumor growth after i.t. injections

| | | | | Number of |
|---|---|---|---|---|
| | | | | tumor free |
| Virus | | | | mice at |
| name | TK locus | RR locus | Dose (pfu) | D 100 |
| COPTG19407 | p7.5K -HC* | p7.5K -LC*; | 7.5 × 10⁶ | 7/10 |
| | | pSE/L-GM-CSF | | |
| COPTG19421 | p7.5K -HC* | p7.5K -LC* | 7.5 × 10⁶ | 5/10 |
| COPTG19347 | pH5.R -HC* | pH5.R -LC* | 7.5 × 10⁶ | 7/10 |
| VVTG18058 | — | — | 7.5 × 10⁶ | 0/10 |
| Mock | | | — | 0/10 |

*HC and LC stand for heavy and light chain of m5-B07 murine anti-mCTLA4 antibody respectively

Moreover the dose escalation performed with both the “empty” virus (VVTG18058) and the COPTG19384 surrogate (COPTG19407) demonstrated that even at a relative low dose (7.5×10⁴ pfu) the antibody expressing virus had still some clear antitumoral activities with 4/10 and 2/10 tumor free mice at 80-98 days versus 0/10 for the treatment with VVTG18058 at the same low dose as shown in the following Table.

TABLE 7
effect of the dose of COPTG19407 or VVTG18058
on the tumor growth after i.t. injections

| | | | | Number of |
|---|---|---|---|---|
| | | | | tumor free |
| Virus | | | | mice at |
| name | TK locus | RR locus | Dose (pfu) | D 100 |
| COPTG19407 | p7.5K -HC* | p7.5K -LC*; | 7.5 × 10⁶ | 7/10 |
| | | pSE/L-GM-CSF | | |
| | | | 7.5 × 10⁵ | 8/10 |
| | | | 7.5 × 10⁴ | 4/10 |
| VVTG18058 | — | — | 7.5 × 10⁶ | 0/10 |
| | | | 7.5 × 10⁵ | 0/10 |
| | | | 7.5 × 10⁴ | 0/10 |

*HC and LC stand for heavy and light chain of m5-B07 murine anti-mCTLA4 antibody respectively

A compilation of survival data (global survival plot compiled from two independent studies) are presented in FIG. 33. A statistical analysis, using logrank test, has been performed to figure out if there were significant differences between survival of each group.

Anti-Tumoral Activity of COPTG19407 Compared to the Combination of VVTG18058 Plus m5-B07

CT26 tumor-bearing mice were set up as described previously (Example 5). Briefly, CT26 cells were injected s.c. into Balb/C mice. The treatment of the mice was started when tumors reached approx. 100 mm³. Mice were then injected at D0, D2 and D5 with COPTG19407 (8.5×10⁶ pfu i.t.), VVTG18058 (8.5×10⁶ pfu i.t.), m5-B07 (10 mg/kg i.p.) or the combination of VVTG18058 (8.5×10⁶ pfu i.t.) plus m5-B07 (10 mg/kg i.p.). Tumor dimensions were then measured twice a week and mice were euthanized when tumors reached 2000 mm³. As shown in FIGS. 34A to 34D, tumor growth was significantly inhibited when mice were treated with the virus COPTG19407 expressing anti-CTLA-4 and GM-CSF whereas the combination of the unarmed virus plus anti-CTLA4 m5-B07 did not result in improved therapy compared to single agent use. In the groups treated with only m5-B07, only virus VVTG18058 or the combination of both m5-B07 and VVTG18058, only 20% of the mice survived after day 70 (FIG. 34E). In contrast, 90% of the mice survived more than 100 days after administration of COPTG19407 demonstrating the potency of the vectorization strategy.

### Antitumoral Activity of VVTK-RR-Encoding Anti-CTLA-4 and GM-CSF in Mice Bearing A20 Subcutaneous Murine B-Cell Lymphoma

The A20 cell line is a BALB/c B cell lymphoma line derived from a spontaneous reticulum cell neoplasm found in an old BALB/cAnN mouse (ATCC TIB-208™).

### Protocol (1):

Tumors were induced by subcutaneous injection of 5×10⁶ of A20 cells into the right flank of female Balb/cN mice (Charles River, France). When tumors reached a mean volume of 95 mm³, 50 mice were randomized into 5 groups of ten animals.
Mice from group 1 received an i.t. administration of vehicle at D0, D2 and D4,
Mice from group 2 received an i.t. administration of VVTG18058 at a dose of 4.75×10⁶ pfu at D0, D2 and D4,
Mice from group 3 received an i.t. administration of COPTG19407 at a dose of 4.75×10⁶ pfu at D0, D2 and D4,
Mice from group 4 received an i.p. administration of anti-PD-1 antibody at a dose of 250 μg at D7, D10, D14, D17, D21 and D24,
Mice from group 5 received an i.t. administration of COPTG19407 at a dose of 4.75×10⁶ pfu at D0, D2 and D4 combined with an i.p. administration of anti-PD-1 antibody at a dose of 250 μg at D7, D10, D14, D17, D21 and D24.

### Antitumoral Activity:

The tumor volumes of all animals was monitored throughout the study. The anti-tumoral activity of treatments is based on the evaluation of the criteria of tumor doubling time, tumor growth delay and tumor growth inhibition (T/C %).

Tumor doubling time was similar for Groups 1, 2 and 4 ranging from 5.14 days (Group 1) to 6.37 days (Group 2). For Group 3, the tumor doubling time could not be precisely calculated since the tumors did not grow exponentially indicating an increased efficacy of treatment, compared to Groups 1, 2 and 4. Similarly, tumor doubling time was calculated using only one animal in Group 5. 9 out of 10 mice in Group 3 had tumors which regressed on D15 and did not grow substantially in volume from D25 until the end of the study on D64. On D64, tumor volumes of these 9 mice ranged from 4 mm³ (technical limit of tumor detection) to 59.77 mm³. Similarly, in Group 5, tumors regressed in 9 mice following the start of treatment, to reach values ranging from 7.24 to 63.21 mm³ at the end of the study on D64. As it can be seen in FIG. 35 presenting the individual tumor volume curves of each group of treatment (Groups 1 to 5 corresponding to FIG. 35A to E), tumors did not grow in animals of groups 3 and 5 receiving COPTG19407 confirming the strong anti-tumor activity of the antibody-expressing virus with or without anti-PD1.

The tumor growth delay was calculated by estimating the time taken for tumors to reach a mean target volume of 300 mm³. The results were similar to those obtained with tumor doubling time, since this parameter could only be calculated for tumors that reached the target volume of 300 mm³, which was not the case for the majority of animals in Groups 3 and 5. Groups 1, 2 and 4 had mean tumor growth delays of 16, 21 and 17 days respectively which were not significantly different to each other. In addition, Groups 3 (n=2) had a mean tumor delay of 14 days, indicated that the tumors that did grow in this group, grew at the same rate as Groups 1, 2 and 4 however these tumors did actually regress in both animals. In comparison, the single tumor in Group 5 (n=1) which grew had a significantly (p≤0.0026) longer tumor growth delay than all other groups, of 43 days.

Tumor growth inhibition (T/C %) was calculated by comparing the median tumor volume of the vehicle treated Group 1 with the other treatment groups. Group 2 had an optimum T/C % of 34% on D22, indicating a transient marginal anti-tumoral activity but this value increased up to 71% by D31. Moderate anti-tumoral activity (10-30% T/C %) was observed in Group 4. In comparison, Groups 3 and 5 both showed marked anti-tumoral activity (T/C % less than 10%) from D27 to D31 (last calculable value of T/C %).

FIG. 36 illustrates the mean tumor volume curves on BALB/cN mice bearing subcutaneous A20 tumors demonstrating the dramatic effect of the anti-CTLA4 and Gm-CSF expressing virus COPTG19407 with or without anti-PD1 on tumor growth.

### Protocol (2):

Tumors were induced by subcutaneous injection of 5×10⁶ of A20 cells into the right flank of female BALB/cN mice. When tumors reached a mean volume of 80-100 mm³, 90 animals were randomized into 9 groups of 10 animals.
Mice from group 1 received an i.t. administration of vehicle at D0, D2 and D4.
Mice from group 2 received an i.p. administration of anti-PD-1 antibody at a dose of 250 μg/mouse/injection at D0, D4, D7, D10, D14 and D17
Mice from group 3 received an i.p. administration of isotype at a dose of 250 μg/mouse/injection at D0, D4, D7, D10, D14 and D17.
Mice from group 4 received an i.t. administration of VVTG18058 at a dose of 1×10⁵ pfu at D0, D2 and D4
Mice from group 5 received an i.t. administration of VVTG18058 at a dose of 1×10⁵ pfu at D0, D2 and D4, and an i.p. administration of isotype at a dose of 250 μg/mouse/injection at D0, D4, D7, D10, D14 and D17
Mice from group 6 received an i.t. administration of VVTG18058 at a dose of 1×10⁵ pfu at D0, D2 and D4 and an i.p. administration of anti-PD-1 antibody at a dose of 250 μg//mouse/injection at D0, D4, D7, D10, D14 and D17
Mice from group 7 received an i.t. administration of COPTG19407 at a dose of 1×10⁵ pfu at D0, D2 and D4.
Mice from group 8 received an i.t. administration of COPTG19407 at a dose of 1×10⁵ pfu at D0, D2 and D4, combined with an i.p. administration of isotype at a dose of 250 μg/mouse/injection at D0, D4, D7, D10, D14, D17 and D24.
Mice from group 9 received an i.t. administration of COPTG19407 at a dose of 1×10⁵ pfu at D0, D2 and D4, combined with an i.p. administration of anti-PD-1 antibody at a dose of 250 μg/mouse/injection at D0, D4, D7, D10, D14 and D17.

### Antitumoral Activity:

The dose of COPTG19407 is suboptimal and demonstrated a mild antitumoral activity similar to the one of the anti-PD-1 treatment in terms of tumor volume and mice survival.

In contrast, the combination of COPTG19407 with anti-PD-1 showed an strong anti-tumoral activity resulting in a small tumor volume compared to the other groups as showed in FIG. 37A (approximately 290 mm³ at Day 36 compared to approximatively 630 mm³ and 750 mm³ at day 24 for mice receiving anti-PD-1 alone or COPTG19407 alone respectively), and a much better survival of the animals as presented in FIG. 37B (7 animals still alive at Day 57 in group 9 versus only two or one in groups 2 or 7 respectively).

### Antitumoral Activity Study of VVTK-RR-Encoding Anti-CTLA-4 and GM-CSF in Mice Bearing C38 Subcutaneous Colon Tumor Cells

C38 is a murine colon adenocarcinoma originating from the American Type Culture Collection (ATCC CRL-2779™).

### Protocol:

Tumors fragments (30-50 mg) were subcutaneously implanted into the right flank of female C57BL/6J mice (Janvier, France). When tumors reached a mean volume of approximately 60 mm³, 50 animals were randomized into five groups of ten animals.
Mice from group 1 received an i.t. administration of vehicle at D0, D2 and D4,
Mice from group 2 received an i.t. administration of an VVTG18058 at a dose of 4.75×10⁶ pfu at D0, D2 and D4,
Mice from group 3 received an i.t. administration of COPTG19407 at a dose of 4.75×10⁶ pfu at D0, D2 and D4,
Mice from group 4 received an i.p. administration of the murine anti-PD-1 antibody at a dose of 250 μg at D7, D10, D14, D17, D21 and D24,
Mice from group 5 received an i.t. administration of COPTG19407 at a dose of 4.75×10⁶ pfu at D0, D2 and D4 combined with an i.p. administration of anti-PD-1 antibody at a dose of 250 μg at D7, D10, D14, D17, D21 and D24.

### Antitumoral Activity:

As before, the tumor volumes of all animals was monitored throughout the study. Tumor doubling time was similar for Groups 1 and 2 at approximately 6.7 days. Group 4 (n=5) had a longer tumor doubling time (10.4 days) but there were no significant differences between groups. For Groups 3 and 5, the tumor doubling time was calculable but with fewer animals (n=2) since the majority of tumors on mice in these groups did not grow exponentially indicating an increased efficacy of treatment, compared to Groups 1, 2 and 4. As it can be seen from FIG. 38, 8 out of 10 mice in Group 3 had tumors which regressed from D15 and did not grow substantially in volume, with five mice having no detectable tumors at the end of the study on D61. Similarly, in Group 5, tumors regressed in 8 mice following the start of treatment, to reach values ranging from 0 (n=2) to 47.82 mm³ at the end of the study on D61.

The tumor growth delay was calculated by estimating the time taken for tumors to reach a mean target volume of 300 mm³. The results were similar to those obtained with tumor doubling time, since this parameter could only be calculated for tumors that reached the target volume of 300 mm³, which was not the case for the majority of animals in Groups 3 and 5. There were no significant differences between groups. Groups 1, 2 and 4 (n=5) had mean tumor growth delays of 23 to 27 days respectively. In addition, Groups 3 (n=2) and 5 (n=3) had mean growth delays of 18 and 24 days, respectively. This indicated that the tumors that did grow in these two groups, grew at similar rates as those in Groups 1, 2 and 4.

Tumor growth inhibition (T/C %) was calculated by comparing the median tumor volume of the vehicle treated Group 1 with the other treatment groups. Group 2 did not show any tumor growth inhibition, since T/C % s remained above 100% for the duration of the study. In comparison, Groups 3, 4 and 5 all showed marked anti-tumoral activity (T/C % less than 10%) from D31 (Group 3 only) to D42 (last calculable value of T/C %).

FIG. 39 illustrates the mean tumor volume curves of C57BL/6 mice bearing subcutaneous C38 tumors demonstrating the dramatic effect of the anti-CTLA4/GM-CSF expressing virus COPTG19407 with or without anti-PD1 on tumor growth.

### Antitumoral Activity Study of VVTK-RR-Encoding Anti-CTLA-4 and GM-CSF in Mice Bearing EMT6 Subcutaneous Breast Tumor Cells

EMT6 is a murine breast carcinoma originating from the ATTC (ATCC CRL-2755™).

### Protocol:

Tumors were induced by subcutaneous injection of 1×10⁶ EMT6 cells in female BALB/cByJ mice (Charles River, France). When tumors reached a mean volume of approximately 51 mm³, fifty mice were randomized by individual tumor volume into five groups of ten animals.
Mice from group 1 received an i.t. administration of vehicle at D0, D2 and D4,
Mice from group 2 received an i.t. administration of VVTG18058 at a dose of 4.75×106 pfu at D0, D2 and D4,
Mice from group 3 received an i.t. administration of COPTG19407 at a dose of 4.75×106 pfu at D0, D2 and D4,
Mice from group 4 received an i.p. administration of anti-PD-1 antibody at a dose of 250 μg at D7, D10, D14, D17, D21 and D24,
Mice from group 5 received an i.t. administration of COPTG19407 at a dose of 4.75×106 pfu at D0, D2 and D4 combined with an i.p. administration of anti-PD-1 antibody at a dose of 250 μg at D7, D10, D14, D17, D21 and D24.

The tumor volumes of all animals was monitored throughout the study by evaluating the criteria of tumor doubling time, tumor growth delay and tumor growth inhibition (T/C %).

### Antitumoral Activity:

Tumor doubling time was similar for Groups 1, 2 and 4 at approximately 5.4 days. For Group 3, the tumor doubling time could not be calculated since the majority of tumors did not grow exponentially indicating an increased efficacy of treatment, compared to Groups 1, 2 and 4. A similar effect was observed in Group 5, where only one animal was used for the calculation of tumor doubling time. As can be seen on the graphs of individual tumor volume (FIG. 40), 8 out of 10 mice in Group 3 had tumors which regressed from D15 and did not grow substantially in volume, with seven mice having no detectable tumors at the end of the study on D61. Similarly, in Group 5, tumors regressed in 9 mice following the start of treatment, to reach values ranging from 0 (n=8) to 13.24 mm³ at the end of the study on D56.

The tumor growth delay was calculated by estimating the time taken for tumors to reach a mean target volume of 200 mm³. The results were similar to those obtained with tumor doubling time, since this parameter could only be calculated for tumors that reached the target volume of 200 mm³, which was not the case for the majority of animals in Groups 3 and 5. Groups 1, 2 and 4 had mean tumor growth delays of approximately 19 days. In addition, Groups 3 and 5 (n=2 for both groups) had mean growth delays of 24 and 12 days, respectively. This indicated that the tumors that did grow in these two groups, grew at similar rates as those in Groups 1, 2 and 4. There were no significant differences between groups.

Tumor growth inhibition (T/C %) was calculated by comparing the median tumor volume of the vehicle treated Group 1 with the other treatment groups. Group 2 showed transient marginal tumor growth inhibition on D28 but increased to 79% on D31. Group 4 showed no anti-tumoral activity, with a T/C %>60% for the duration of the study. In comparison, Groups 3 and 5 both showed marked anti-tumoral activity (T/C % less than 10%) from D24 to D31 (last calculable value of T/C %).

FIG. 41 illustrates the mean tumor volume curves on BALB/cByJ mice bearing subcutaneous EMT6 tumors demonstrating the dramatic effect of the anti-CTLA4/GM-CSF expressing virus COPTG19407 with or without anti-PD1 on tumor growth.

### CT26 Rechallenge

Balb/c mice challenged with CT26 tumor cells who survived after treatment with 10⁴, 10⁵ or 10⁶ pfu of COPTG19421 or 10⁶ pfu of COPTG19407 were rechallenged with CT26 tumor cells or challenged with Renca cells (renal adenocarcinoma cells: control), in order to study if a specific antitumoral immune response was raised.

TABLE 8
Effect of the rechallenge with CT26 tumor cells or challenge
with Renca tumor cells on the number of tumor free mice

| Group | Tumor free/Total mice |
|---|---|
| CT26 control naïve | 0/5 |
| VVTG18058 10⁵ + CT26 rechallenge | 0/1 |
| COPTG19421 10⁶ + CT26 rechallenge | 3/3 |
| COPTG19421 10⁶ + RenCa challenge | 0/2 |
| COPTG19407 10⁴ + CT26 rechallenge | 1/1 |
| COPTG19407 10⁴ + RenCa challenge | 0/1 |
| COPTG19407 10⁵ + CT26 rechallenge | 1/2 |
| COPTG19407 10⁵ + RenCa challenge | 0/2 |
| COPTG19407 10⁶ + CT26 rechallenge | 2/4 |
| COPTG19407 10⁶ + RenCa challenge | 0/3 |

Results presented in table 8 show that 0/8 mice having received COPTG19421 or COPTG19407 were tumor free after RenCa challenge, while 7/10 mice having received COPTG19421 or COPTG19407 were tumor free after CT26 rechallenge. This indicates that COPTG19421 and COPTG19407 raised a specific immune memory against CT26 cells.

1-34. (canceled)

35. An isolated nucleotide sequence encoding an antibody molecule that specifically binds to CTLA-4, wherein the antibody molecule comprises the 6 CDRs having VH-CDR1 of SEQ ID NO: 15, the VH-CDR2 of SEQ ID NO: 16, the VH-CDR3 of SEQ ID NO: 17, the VL-CDR1 of SEQ ID NO: 10, the VL-CDR2 of SEQ ID NO: 18, and the VL-CDR3 of SEQ ID NO: 19, or comprises the 6 CDRs having VH-CDR1 of SEQ ID NO: 22, the VH-CDR2 of SEQ ID NO: 23, the VH-CDR3 of SEQ ID NO: 24, the VL-CDR1 of SEQ ID NO: 10, the VL-CDR2 of SEQ ID NO: 25, and the VL-CDR3 of SEQ ID NO: 26.

36. The isolated nucleotide sequence according to claim 1, comprising or consisting of a sequence selected from the group consisting of SEQ ID NOs: 45-48.

37. A plasmid, virus or cell comprising a nucleotide sequence as defined in claim 1.

38. A plasmid, virus or cell comprising a nucleotide sequence as defined in claim 2.

39. The plasmid, virus or cell according to claim 3, which is an oncolytic virus, such as an oncolytic poxvirus.

40. The plasmid, virus or cell according to claim 4, which is an oncolytic virus, such as an oncolytic poxvirus.

41. The plasmid, virus or cell according to claim 5, wherein said poxvirus belongs to the Chordopoxviridae subfamily, more preferably to the Orthopoxvirus genus preferably selected from the group consisting of Vaccinia virus, cowpox virus, canarypox virus, ectromelia virus and myxoma virus.

42. The plasmid, virus or cell according to claim 6, wherein said poxvirus belongs to the Chordopoxviridae subfamily, more preferably to the Orthopoxvirus genus preferably selected from the group consisting of Vaccinia virus, cowpox virus, canarypox virus, ectromelia virus and myxoma virus.

43. The plasmid, virus or cell according to claim 7, wherein the poxvirus is a Vaccinia virus.

44. The plasmid, virus or cell according to claim 8, wherein the poxvirus is a Vaccinia virus.

45. The plasmid, virus or cell according to claim 7, wherein said oncolytic virus is a vaccinia virus defective for both thymidine kinase (TK) and/or ribonucleotide reductase (RR) activities and comprising nucleotide sequences encoding SEQ ID NO: 20 and SEQ ID NO: 21 or SEQ ID NO: 53 and SEQ ID NO: 54.

46. The plasmid, virus or cell according to claim 8, wherein said oncolytic virus is a vaccinia virus defective for both thymidine kinase (TK) and/or ribonucleotide reductase (RR) activities and comprising nucleotide sequences encoding SEQ ID NO: 20 and SEQ ID NO: 21 or SEQ ID NO: 53 and SEQ ID NO: 54.

47. The plasmid, virus or cell according to claim 11, wherein said oncolytic vaccinia virus further comprises a nucleotide sequence encoding a GM-CSF, with a specific preference for a human GM-CSF (e.g. having SEQ ID NO: 55 or SEQ ID NO: 56) or a murine GM-CSF (e.g. having SEQ ID NO: 57 or SEQ ID NO: 58).

48. The plasmid, virus or cell according to claim 12, wherein said oncolytic vaccinia virus further comprises a nucleotide sequence encoding a GM-CSF, with a specific preference for a human GM-CSF (e.g. having SEQ ID NO: 55 or SEQ ID NO: 56) or a murine GM-CSF (e.g. having SEQ ID NO: 57 or SEQ ID NO: 58).

49. The plasmid, virus or cell according to claim 3, wherein the cassette encoding the heavy chain is inserted at the J2R locus and the cassette encoding the light chain is inserted at the I4L locus.

50. The plasmid, virus or cell according to claim 4, wherein the cassette encoding the heavy chain is inserted at the J2R locus and the cassette encoding the light chain is inserted at the I4L locus.

51. A pharmaceutical composition comprising the nucleotide sequence according to claim 1, and optionally a pharmaceutically acceptable diluent, carrier, vehicle and/or excipient.

52. A method for treatment of cancer, such as a solid cancer, in a subject comprising administering to the subject a therapeutically effective amount of the isolated nucleotide sequence as defined in claim 1.

53. A method for treatment of cancer in a subject comprising administering to the subject a therapeutically effective amount of the plasmid, virus or cell as defined in claim 3.

54. A method for treatment of cancer, such as a solid cancer, in a subject comprising administering to the subject a therapeutically effective amount of the plasmid, virus or cell as defined in claim 4.


SEQUENCE LISTING (63 sequences)

SEQ ID NO: 1 - protein, 14 aa, Homo sapiens
  VARIANT 2: note = S or K
  VARIANT 3: note = D, S, or A
  VARIANT 5: note = Y, S, or A
  VARIANT 7: note = S or N
  VARIANT 9: note = V or I
FXXYXMXWXRQAPG

SEQ ID NO: 2 - protein, 14 aa, Homo sapiens
  VARIANT 3: note = D, or S
  VARIANT 5: note = Y, S, or A
  VARIANT 7: note = S, or N
FSXYXMXWVRQAPG

SEQ ID NO: 3 - protein, 14 aa, Homo sapiens
  VARIANT 3: note = D, or S
  VARIANT 5: note = Y, or S
  VARIANT 7: note = S, or N
FSXYXMXWVRQAPG

SEQ ID NO: 4 - protein, 19 aa, Homo sapiens
  VARIANT 2: note = G, or A
  VARIANT 5: note = W, G, or N
  VARIANT 6: note = S, or T
  VARIANT 7: note = S, or G
  VARIANT 8: note = R, or G
  VARIANT 9: note = D, S, or Y
  VARIANT 10: note = K, T, or I
  VARIANT 11: note = G, Y, H, or D
  VARIANT 12: note = Y, or F
SXISXXXXXXXXADSVKGR

SEQ ID NO: 5 - protein, 19 aa, Homo sapiens
  VARIANT 2: note = G, or A
  VARIANT 5: note = W, G, or N
  VARIANT 6: note = S, or T
  VARIANT 7: note = S, or G
  VARIANT 8: note = R, or G
  VARIANT 9: note = D, S, or Y
  VARIANT 10: note = K, T, or I
  VARIANT 11: note = G, Y, H, or D
SXISXXXXXXXYADSVKGR

SEQ ID NO: 6 - protein, 19 aa, Homo sapiens
  VARIANT 2: note = G, or A
  VARIANT 5: note = W, or G
  VARIANT 7: note = S, or G
  VARIANT 8: note = R, or G
  VARIANT 9: note = D, or S
  VARIANT 10: note = K, or T
  VARIANT 11: note = G, Y, H, or D
SXISXSXXXXXYADSVKGR

SEQ ID NO: 7-8 - skipped (2 entries)

SEQ ID NO: 9 - protein, 15 aa, Homo sapiens
  VARIANT 2: note = T, or S
  VARIANT 10: note = A, or S
  VARIANT 11: note = G, or N
  VARIANT 13: note = D, orV
  VARIANT 14: note = V, or Y
  VARIANT 15: note = H, or none
CXGSSSNIGXXYXXX

SEQ ID NO: 10 - protein, 15 aa, Homo sapiens
CTGSSSNIGAGYDVH

SEQ ID NO: 11 - protein, 7 aa, Homo sapiens
  VARIANT 1: note = G, R, or D
  VARIANT 3: note = D, N, or S
  VARIANT 4: note = N, Q, or K
XNXXRPS

SEQ ID NO: 12 - protein, 7 aa, Homo sapiens
  VARIANT 1: note = G, or R
  VARIANT 3: note = D, or N
  VARIANT 4: note = N, or Q
XNXXRPS

SEQ ID NO: 13 - protein, 13 aa, Homo sapiens
  VARIANT 2: note = A, or Q
  VARIANT 3: note = V, A, or S
  VARIANT 4: note = W, or Y
  VARIANT 6: note = D, or S
  VARIANT 9: note = N, or S
  VARIANT 11: note = V, W, or P
  VARIANT 13: note = V, or none
CXXXDXSLXGXVX

SEQ ID NO: 14 - protein, 12 aa, Homo sapiens
  VARIANT 3: note = V, or A
  VARIANT 11: note = V, or W
CAXWDDSLNGXV

SEQ ID NO: 15 - protein, 14 aa, Homo sapiens
FSDYYMSWVRQAPG

SEQ ID NO: 16 - protein, 19 aa, Homo sapiens
SGISWSSRDKGYADSVKGR

SEQ ID NO: 17 - protein, 7 aa, Homo sapiens
TTDLARY

SEQ ID NO: 18 - protein, 7 aa, Homo sapiens
GNDNRPS

SEQ ID NO: 19 - protein, 12 aa, Homo sapiens
CAVWDDSLNGVV

SEQ ID NO: 20 - protein, 114 aa, Homo sapiens
EVQLLESGGGLVQPGGSLRLSCAASGFTFSDYYMSWVRQAPGKGLEWVSGISWSSRDKGYADSVKGRFTISRDNSKNTLYLQMNSLRAEDTAVYYCTTDLARYWGQGTLVTVSS

SEQ ID NO: 21 - protein, 112 aa, Homo sapiens
QSVLTQPPSASGTPGQRVTISCTGSSSNIGAGYDVHWYQQLPGTAPKLLIYGNDNRPSGVPDRFSGSKSGTSASLAISGLRSEDEADYYCAVWDDSLNGVVFGGGTKLTVLG

SEQ ID NO: 22 - protein, 14 aa, Homo sapiens
FSSYSMNWVRQAPG

SEQ ID NO: 23 - protein, 19 aa, Homo sapiens
SAISGSGGSTYYADSVKGR

SEQ ID NO: 24 - protein, 13 aa, Homo sapiens
ARDRVEMNQWLAD

SEQ ID NO: 25 - protein, 7 aa, Homo sapiens
RNNQRPS

SEQ ID NO: 26 - protein, 12 aa, Homo sapiens
CAAWDDSLNGWV

SEQ ID NO: 27 - protein, 120 aa, Homo sapiens
EVQLLESGGGLVQPGGSLRLSCAASGFTFSSYSMNWVRQAPGKGLEWVSAISGSGGSTYYADSVKGRFTISRDNSKNTLYLQMNSLRAEDTAVYYCARDRVEMNQWLADWGQGTLVTVSS

SEQ ID NO: 28 - protein, 112 aa, Homo sapiens
QSVLTQPPSASGTPGQRVTISCTGSSSNIGAGYDVHWYQQLPGTAPKLLIYRNNQRPSGVPDRFSGSKSGTSASLAISGLRSEDEADYYCAAWDDSLNGWVFGGGTKLTVLG

SEQ ID NO: 29 - protein, 14 aa, Homo sapiens
FSSYAMSWVRQAPG

SEQ ID NO: 30 - protein, 19 aa, Homo sapiens
SGISGSGGYIHYADSVKGR

SEQ ID NO: 31 - protein, 13 aa, Homo sapiens
ATYSSGLHDAFDI

SEQ ID NO: 32 - protein, 7 aa, Homo sapiens
DNNKRPS

SEQ ID NO: 33 - protein, 120 aa, Homo sapiens
EVQLLESGGGLVQPGGSLRLSCAASGFTFSSYAMSWVRQAPGKGLEWVSGISGSGGYIHYADSVKGRFTISRDNSKNTLYLQMNSLRAEDTAVYYCATYSSGLHDAFDIWGQGTLVTVSS

SEQ ID NO: 34 - protein, 112 aa, Homo sapiens
QSVLTQPPSASGTPGQRVTISCTGSSSNIGAGYDVHWYQQLPGTAPKLLIYDNNKRPSGVPDRFSGSKSGTSASLAISGLRSEDEADYYCAAWDDSLNGWVFGGGTKLTVLG

SEQ ID NO: 35 - protein, 14 aa, Homo sapiens
FKAYSMSWIRQAPG

SEQ ID NO: 36 - protein, 19 aa, Homo sapiens
SGISNTGGSTDFADSVKGR

SEQ ID NO: 37 - protein, 15 aa, Homo sapiens
ARLGYSGYDDRGMDV

SEQ ID NO: 38 - protein, 14 aa, Homo sapiens
CSGSSSNIGSNYVY

SEQ ID NO: 39 - protein, 7 aa, Homo sapiens
GNSNRPS

SEQ ID NO: 40 - protein, 13 aa, Homo sapiens [sequence omitted]

SEQ ID NO: 41 - protein, 122 aa, Homo sapiens
EVQLLESGGGLVQPGGSLRLSCAASGFTFKAYSMSWIRQAPGKGLEWVSGISNTGGSTDFADSVKGRFTISRDNSKNTLYLQMNSLRAEDTAMYYCARLGYSGYDDRGMDVWGQGTLVTVSS

SEQ ID NO: 42 - protein, 112 aa, Homo sapiens
QSVLTQPPSASGTPGQRVTISCSGSSSNIGSNYVYWYQQLPGTAPKLLIYGNSNRPSGVPDRFSGSKSGTSASLAISGLRSEDEADYYCQSYDSSLSGPVVFGGGTKLTVLG

SEQ ID NO: 43 - protein, 330 aa, Homo sapiens [sequence omitted]

SEQ ID NO: 44 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 45 - DNA, 1335 bp, Homo sapiens
  source 1..1335: mol_type = other DNA
gaggtgcagctgttggagtctgggggaggcttggtacagcctggggggtccctgagactctcctgtgcagcctctggattcaccttcagtgactactacatgagctgggtccgccaggctcccgggaaggggctggagtgggtctcaggcattagttggagtagtcgtgacaaaggctatgcggactctgtgaagggccgtttcaccatctccagagacaattccaagaacacgctgtatctgcaaatgaacagcctgagagccgaggacactgccgtgtattactgtaccacagatctcgctaggtactggggccagggtacactggtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 46 - DNA, 654 bp, Homo sapiens
  source 1..654: mol_type = other DNA
cagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctatggtaatgataaccggccctcaggggtccctgaccgattctctggctccaagtctggcacctcagcctccctggccatcagtgggctccggtccgaggatgaggctgattattactgtgcagtatgggatgacagcctgaatggtgtggtattcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 47 - DNA, 1353 bp, Homo sapiens
  source 1..1353: mol_type = other DNA
gaggtgcagctgttggagtctgggggaggcttggtacagcctggggggtccctgagactctcctgtgcagcctctggattcaccttcagtagctatagcatgaactgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacactgccgtgtattactgtgcgagagatcgggtagagatgaaccagtggctggccgactggggccagggtacactggtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 48 - DNA, 654 bp, Homo sapiens
  source 1..654: mol_type = other DNA
cagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgaatggttgggtgttcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 49 - DNA, 1353 bp, Homo sapiens
  source 1..1353: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattagtggcagcggtgggtacatacactatgcagactccgtgaagggccggttcaccatctccagagacaattccaagaacacgctgtatctgcaaatgaacagcctgagagccgaggacactgccgtgtattactgtgcgacctatagcagtggcctgcatgatgcttttgatatctggggccagggtacactggtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 50 - DNA, 654 bp, Homo sapiens
  source 1..654: mol_type = other DNA
cagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctatgacaataataagcgaccctcaggggtccctgaccgattctctggctccaagtctggcacctcagcctccctggccatcagtgggctccggtccgaggatgaggctgattattactgtgcagcatgggatgacagcctgaatggttgggtgttcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 51 - DNA, 1359 bp, Homo sapiens
  source 1..1359: mol_type = other DNA
gaggtgcagctgttggagtctgggggaggcttggtacagcctggggggtccctgagactctcctgtgcagcctctggattcaccttcaaagcctatagcatgagctggatccgccaggctccagggaaggggctggagtgggtctcaggtatcagtaacacgggaggtagcacagacttcgcagactccgtgaagggccggttcaccatctccagagacaattccaagaacacgctgtatctgcaaatgaacagcctgagagccgaggacactgccatgtattactgtgcgagattgggatatagtggctacgacgaccgtggtatggacgtctggggccaaggtacactggtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 52 - DNA, 654 bp, Homo sapiens
  source 1..654: mol_type = other DNA
cagtctgtgctgactcagccaccctcagcgtctgggacccccgggcagagggtcaccatctcttgttctggaagcagctccaacatcggaagtaattatgtgtactggtatcagcagctcccaggaacggcccccaaactcctcatctatggtaacagcaatcggccctcaggggtccctgaccgattctctggctccaagtctggcacctcagcctccctggccatcagtgggctccggtccgaggatgaggctgattattactgccagtcctatgacagcagcctgagtggtcctgtggtattcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 53 - protein, 236 aa, Homo sapiens
MGWSCIILFLVATATGVHSQSVLTQPPSASGTPGQRVTISCTGSSSNIGAGYDVHWYQQLPGTAPKLLIYGNDNRPSGVPDRFSGSKSGTSASLAISGLRSEDEADYYCAVWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 54 - protein, 463 aa, Homo sapiens
MGWSCIILFLVATATGVHSEVQLLESGGGLVQPGGSLRLSCAASGFTFSDYYMSWVRQAPGKGLEWVSGISWSSRDKGYADSVKGRFTISRDNSKNTLYLQMNSLRAEDTAVYYCTTDLARYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 127 aa, Homo sapiens
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQTITFESFKENLKDFLLVIPFDCWEPVQE

SEQ ID NO: 56 - protein, 144 aa, Homo sapiens [sequence omitted]

SEQ ID NO: 57 - protein, 124 aa, Mus musculus
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 58 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 59 - DNA, 124 bp, synthetic construct
  regulatory 1..124: note = Promoter p7.5K; regulatory_class = promoter
  source 1..124: mol_type = other DNA
ccacccactttttatagtaagtttttcacccataaataataaatacaataattaatttctcgtaaaagtagaaaatatattctaatttattgcacggtaaggaagtagaatcataaagaacagt

SEQ ID NO: 60 - DNA, 114 bp, synthetic construct
  regulatory 1..114: note = Promoter pH5.R; regulatory_class = promoter
  source 1..114: mol_type = other DNA
tttattctatacttaaaaaatgaaaataaatacaaaggttcttgagggttgtgttaaattgaaagcgagaaataatcataaattatttcattatcgcgatatccgttaagtttg

SEQ ID NO: 61 - DNA, 40 bp, synthetic construct
  regulatory 1..40: note = Promoter pSE/L; regulatory_class = promoter
  source 1..40: mol_type = other DNA
aaaaattgaaattttattttttttttttggaatataaata

SEQ ID NO: 62 - protein, 236 aa, Mus musculus
MGWSCIILFLVATATGVHSQSVLTQPPSASGTPGQRVTISCTGSSSNIGAGYDVHWYQQLPGTAPKLLIYRNNQRPSGVPDRFSGSKSGTSASLAISGLRSEDEADYYCAAWDDSLNGWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 63 - protein, 469 aa, Mus musculus
MGWSCIILFLVATATGVHSEVQLLESGGGLVQPGGSLRLSCAASGFTFSSYSMNWVRQAPGKGLEWVSAISGSGGSTYYADSVKGRFTISRDNSKNTLYLQMNSLRAEDTAVYYCARDRVEMNQWLADWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK